SEQUENCE LISTING (91 sequences)

SEQ ID NO: 1 - DNA, 9842 bp, Helicobacter suis
  CDS (420)..(9398)
  sig_peptide (420)..(500)
gttataaccttgaacaaataggctttcacagggcaaaaaatacatgttttctctcacatggaatgcgcctcttgaggctttcacagataaaaatcagttttttggaggtgtaggagtagatggggtgtatctgcatttgcataaagcccatgaattcttaggcatgcgtgctctgccaacttttattgtaaatgacatcattaaaaatccacagggggaatcctatctcaaagattacagcgcgcatttaaaacaggtttttcataaataggcttctttctttttacttcattaactttaaagtttgctagctggatatttttaacaggcgtttttataacgcctcttgtgtatttaaactaaaataagtatctcttaaattaatttcttatacatgattttatgctaaagttttctctatgaaaaagtttagttctctcacattgaaatttggccacgctctagcacggcgcattaaagctaaccaagctaggcgtgcgggtacttatgtctttaaaaaacaactcccccaacccaagcgttctgagttgaaacctaaattaatcaagcaggggacttttattttaggtgtaatgagccaaccgcttttagcgtggtatccttcatggattagtggcacacacacactcaacacaacaaatattaagcagtatatgcaaaataaccatagaagccaaaattggttatggactggtggctctaatgtcttttatgaagctagtaatggaagttatttttgtactaactggaattgtaacggctcggttacattaattggtagtggctctaccacttacacgcttagcaatctcaattacgaaggcggtagcctcaatctacaaattaatggtaatggcacacaaggcgcgcttaatattagcaatgtcaacatggatagctatcaaggaaggcaatttacagatacatggaatgcccaaagtgttagtatcagcggaaatctacaagtagggcaaaatcatatcactatcaacgcaaaccacggcactacagcgaacaacgctaccattaacgcggatcaaggcaacgggattttaaatatcaatgataaaacaagcgagagtttcacaaacaccacctttaaaggtacggggcagatcaatttacagagcaacaacatcacctttaacaatgttacttttaatgatagcaacactggttcacatgttacggacaacggcactcttacccttgagggcactgagacctttgcacaaaactcgccccttatcaatctaggcgccaatgttactatccaagccaacaccatttttaacattacagaagatcttacaaagaccaccaacggagcctataatcttgatacccttgtaagtacaagtggtaataaaagtatcaacgatagtagctatgcaagccatttgtgggatcttatccgctatcaaggccaaacaggtagcctttttaacgggcaactcagtaatggcactactttaagtaatcctagctctggcaatgggatttactatgtgaaatatacttttggcaatggtgattgggatattcttaaagaagattttgaaaataactctctatcggcgcaacttgaggcttacgctaaaaatcagggtgatatttggagtacaatccacaacattaattctacttggaattttaatgtcggagagggtagtgcctatatcaaccccgggaatggaacagatcaagcttgggcacaaagcgataaaaattttaaagttactttagataatgggagtggtgggacgcttattcttggtaatagcacagaaacccccagttcaagcggtaaaattctctttggaggcacagggggtaatccttttgctttaaatggcaattacggcggagatcttggctatatgacaggggaatttgacgcgggtaaaatttatcttaccggtaccatagaaagcggaaactcttttcacgatggtaacggcactaacattagctacaacgctattacaaacattacagctaatggtttgcactatctcaacgataacgccggagcatggcatagcaacgctaattttagagctaccaatggcagtatcaacatctcaaactcccaattccaagatcaaagtagcgggcaatttacctttaactctcaaaatcaaacttttagtagcacaacttttactggcaatagtcatacgatcactctacacgctaccaacaacctaaccctcaccaacacaagctttaatgattcaaatgctagcttgacactagaggcagataagggtagtttacgagataccgataaccaaactagccaaatcacagctaaaaacctccaagtaattgctaatcaagctagttttagtaacacgatctttaatgcacaaaacagctcttttaaaactaaccaattgaccctgacaaatgatacatttaataatggtagctatagctttgcacctgaaaacaacggcaaccacacaaccacatttggcggtaccaccacgattaatactagcagtagcccctttgctaatttaggcggaagcattagtttaaacaacggcgctatttttaatcttaataatattttaagttctttacaaattggtacaacttataacatcttaggcggaagcggggctaatattctttacaagaacgatacacaatatgcctcagcactttggcaactcattaaaatcaacgggactactattaactcagaaacagagataagtgataataacgatacgcaagtttggaatgtagtctttaacatagatggcatgcccattaagatacaagaaacctttgctagtagtggacttagctttaaagttattagccaagctaaagatatttggtcagatgtgtatcacatgactactaattgtgcatacaatgccttagccagcccaccgggctgttatggttatcaaaatggtacacacaacatattgaagcactttaataagtacggctttgaagcttacaatgaaagtgtaggcgcagacgggattgcctatatcaaccctttaaactcacaaagccacgatggctataacgcctccacccacacactccaaacttatacaaaaattggcaatggcggtacttataatgttggggagatgcaaaatggcaaatgggttaacaatggtactttaattttaggtaataacacctcacaacccgttaaagggggtaaaatagaatttggcaaagtggactcagtaggctatatcaccgatgtttttaatgccggacatatttttctaactaacagcatagaagttggggatagttccttaagcggggcgggggcaactctaacttttaatgctaataataatatcaccgccgacggtcttacctaccaccaagatgctaccgctatccctcccttcggctctgctatcagccagcatagctacggtaacttcatcgcacaacaaagctttagcgcgctcaattcctcttttgaagatgatactagcggtagtttagactttaccggtaaaaacagcattaattttacaagcagcaccgttataggtagtaaaagtagtattacccttaactccgcaaataccaccctaaacaactctgccttttttgtgggcaatggtacgactcttacttttggtaatgtgatcacaactaatagtcatagtagctattcatctagcacaaacaccattaataactctacaatcaacctcaaccaaaactctagcttgtatttgcatggcagtaccaccctagataacaccacttcctttttaaacttaagtagtcaagcaactttttatgattcagctagcctaagtggtagcactacaattaatcttgatcacaatagcaagattacaatgaatagcaccactacgctaaatgataacgctaattttaatttaattaactccgctcaagctaactttcaaggcaatagtacttttaataataactctgggatcactcttgctagtggtgctaaggctatgtttacacacactacaacaagcacccaagatattacaacttttaataataactcgtttttaaatgtcaatgggagttttacagattttatacccagtacaaatagtaacaggacaattagcgggggaggatcaagtagtaatcaaaactatagcgcgcagtttaatgatctcgtgtttaacaactatgcttccatgctcttaaatagcgcagatattcaaagtagccaaaccacctttgctaattctgtgaatatcaatatgcaaaatagcctttttaatgcgagtacgcttaatttaaatggcggtaatttttacatgcaaaatagccagattaaagcgggggcagtgagtgtgggatcttctgattctgttaatattaattctggggtcaattctcttaatcgctccctcatcactagctcaagctttaatcttaatggaacattaaacctagatggccttttacttggacctacaactacaggtacaaccaaaagtacggccaacgggcaacctcttatttctgtaggagggggaacatttagcttaaatggcattcttaatatctctaatattgatctctccgcccacctctctagccaaacaaatcacactagttcctctgctacctatgatattgtgaatgctaacaacattacaggcatgagtggggctaatggttatcaaaaaattgagtattacggcattaaaatcaataatgctacctattctgattcaaattctgattcaaataaaacccaaagctggagttttacaaaccctttaaatggcgcacaaactattactgaaaaaatacaaagtggtaaactcactatcaccattagtaatagcaatcattttgtagctacagattaccacaacatcgcccccgaactctttttttacaaacaatccgcacaaaacttgcctagtacaaactcagcaagtgcagatataagcagttatgattattccagcgatgaaagcgggactttctttttagatggcaatttaaaaggggtgtattatccaaaatctagcacaacaggcactaccccagttattccgggtacttataacgctcaaggccagcccttacaaggtctttatatttctaataacggcctgtttaatgaaaccacccttaataacttagtagacattgtccgaagtatttggcctcatttaaaaacccttttgcctaaaattctacaagatttaagcgatccaagtaatatcattcaagacttagaaaactcagggattaaattaactagccagcaaagcaaggaacttttaagttttatagacgggctatcaagcaatatcaaccaaacttttaataatggtacgcttgtagtgggttcgcctcaggcaggacaaaccggaagtagtagcgtggtgtggtttggtggcaatggttatactacggcttgtacggctgcacaaactgaaaaagggtgtcaggatttaagaggcacttatttaggtcagcttttaggatcaacttctgctgcactaggctatattgaggctaattttaaggctaaagatatttatatcaccggcaccgtaggcagtgggaatgcttgggggataggtgggagcgcagatgtaacttttaatagcgcgactaatttaaccctcaatcaagccacgattgatgcggaaggcacggatcaaatttttaatatgctaggtgagggcgggatacaaaaaatcttaggccaaaaggggctagcccagatgttaggtaattatatctacgatctagccaatggctctagtattaatcttaatcccattagtagcattccaagctctatccagcctctggctaaagatttaggcaggcagattggtaaaatcaaacttagcgatgtgcttagcgcctcagatgtgagtgcacttttaaacatgcccggtatggataatgtgatcaaaaatattttaagtactaagactgtgagttctgtattaggcagtcgtgggcttatttctagtcttaaccaatctgagcaaaataaaatctatggtgctattgataaagagttacatttctcagggggaaaagtcattgcctccattgccaacggggtttatggtaaccacacccttttgagcctcattaactccctctctcctatgacgggtaaaaatgtggacaatattttaaacatgccaagcaccacgagcggacaaagccagatcaaaagtcttttagagcaaaccacatttgagcaggtttttcaagaactgctctctaatcaaaacctactcaataaaaccattgcttggttaggcccacaaattttagatgaaatgcttaaaactgctattgatgatttactcaaccctacaaatcagcttactgccgctgaaaaaaatattcttgataacattcttaacaatgtctttagctcagaaaaaaaggcagtccaacaaatggaaaattctaaccctgatgtcaaacacgttattgatggcttaatgcaggctaaaggtctaggggaggtttatagcaagggccttcagagtattttatctaataaactgcaaggtcaattaaaaagcatgggcttaggttctttgctcgcgcctaaagctctaggtaatttctggcagaagggttattttaacttcctagccaatgatgatattttagtgaacaatagcacttttagtaatgcttcaggcgggactttaagttttatagccggtaagtctattatttttgcagggcaaaatacaattaattttagtaataatcaaggtacactagaatttttaagtaatgatgtgtctaatattgatctgactactcttaatgccactgacggtttaaccattgatgccccctttaataatctttatgttcagaaaggcaatatcacccttaatcaatatgagagttttagcgtgcaggcgggcaattttgactttttaggcacggtgcaagcagacggggctgtggatttatccggtgtaaccggtttagctgttttaggtactcttaatcttactgaggatagcacccttaaggctaataatttaaccacaataagcgcctttaacaaccaatcccaaaacctgcttaatatcagcgggaattttaactcctatggcacatttagtacacaaggggctgggataaatatcggaggggggtttaatagcataggggctttaacttttaatgtagcgggtgcaacagtcaaaactacaggccctagctcagatagttcaagttcaagcactagctcctctacaagttctagtaattctaatagctctggaggctctagctcctcctctagtacttcagactctactagttctagcgcgcctactagttctacaacagctacagctttaactctagctagcaccaccgtgtctacaacttctactacaaattctagcagtgatacaagtaatagctcaagctctaatagctcctcttctaatagtgtgtctaatgcaattccggttagccccactcctagtacacaaattccgcttatccaagtaggtggacttgtgaatttaaatttaggtggtagtgcgattattagctttaatacagaggcacaaacaaatagcacgggaactacaggaactacacaaggcacagcaaacacaggcagtaccggatcaagcacaaactctagctctacaagcacatcacaaacaacatctagtagttctactagctccacacaaacaacaagcctaagctctaatactagtctagctactaccagccaaacgcctacaacttctagcggggcttctccggatagttcaaaccctacagcctctcctataaccccttcaaatggcgcttatacgctaattcaaactaatagctggattcattacaaccccacctcctttaatccaaacaactggagacaatatttagagctctacaccagtcttaaaatcaacgggacagctttccagcttaacgctcaaggtacaggtcttacttataatggtcaagcagtcaatatctcacaacgaggcttgcttgtcaattatcaaggcacaaatggccaagaagtgagcgcctctattgattataataagatgcaaataggcataggccaaagcttgcatgttatcgcgcccactattacacaatacatcacccaaatacaagggcagtctgtggttaatgcgctagaaaatgcaggcgggccgggtgtgatgaattggtttggtaagcttttaattgagacaaaaaacaccccgctttttgcgccctactatcttgaacaacactccttaagcgatctacttaaaattgtaaaagatattcaaaatgcgactgattggatgggcgcttctggtttaaaagccactagctctaaattgctacaaatcagtgtacacaccaaacagatgagccgtttagctaagctttcaaattttgcttctaatgatgcattaccagattttcatgattttttagtcagtcttaaaggtaaaaaatttgctagcgcggtgcctaatgctatggatattatcaccgcctattctcaaagagataaattaaaaaataacctatggataaccggtgtgggaggagcaagttttgtagcaggaggcacaggtacgctttatgggctgaatgtgggttatgatcgctttataaagggcgtgattgtggggggttatatggcttatggttatagtggcttttatggcaatatcaatagcgctaattctaataatgtcaatgtgggattttatagccgcgcctttatcaagggtagaaacgagattacaggaagtattaatgaaacctatggctataacaaaacctacattgatgcaactaatcccattctcacccctcttaaccagcaataccactatggcacttggactaccaatgtcggcgctaactatggctatgacttcttttttaaaaacaaacatgttattctcaaaccccaaattggcctcacttattattatatcggcctctcaggcttgcaaggcaagatgaatgatccgatttataacgagtttagagccaatgccgatccagcgcataaatctgttttgacgatcaatttagccctagagagtcgccactattttaggaaaaactcctattactatgtcattgccggcttagggcgggatttattcgtccattctatgggtgataaaatagtacgctttattggtaatgatatgttaagttatcgttatgggggaatgtataatacctttgctagcctcaccacagggggtgaggttcgcttatttcgatccttttatgtcaatgctggtattggagcgcgctttggtttggattatcaagatattaatattacaggcaatgtcgggatgcgctatgctttttagacaaatttttaattctgtgttgctgtctcactttattttaaaattctttttaagctctgtttaagtttttgcgtgctactatccaaatccccctcctttttagtctctggagtttctccagggactttccttgaataatgctaaggctttctttaagacgccaagaacctattttgcgcgtacatgttttacgcaagccaaccctttcttctgaactcttgcttactttagaacaactattaaaagaagccaaaattacttatgattttaaactccctgaaatgtccttgtctcaagaagttttattgtgttttggaggagatggcacgcttctagcggccctgcgccacccttcaaatagcctatgttttggaatccatgtcgggcatttggggtttttaacagccactaatttagaaggcgctccccattttttagaagcgc

SEQ ID NO: 2 - protein, 2992 aa, Helicobacter suis
MKKFSSLTLKFGHALARRIKANQARRAGTYVFKKQLPQPKRSELKPKLIKQGTFILGVMSQPLLAWYPSWISGTHTLNTTNIKQYMQNNHRSQNWLWTGGSNVFYEASNGSYFCTNWNCNGSVTLIGSGSTTYTLSNLNYEGGSLNLQINGNGTQGALNISNVNMDSYQGRQFTDTWNAQSVSISGNLQVGQNHITINANHGTTANNATINADQGNGILNINDKTSESFTNTTFKGTGQINLQSNNITFNNVTFNDSNTGSHVTDNGTLTLEGTETFAQNSPLINLGANVTIQANTIFNITEDLTKTTNGAYNLDTLVSTSGNKSINDSSYASHLWDLIRYQGQTGSLFNGQLSNGTTLSNPSSGNGIYYVKYTFGNGDWDILKEDFENNSLSAQLEAYAKNQGDIWSTIHNINSTWNFNVGEGSAYINPGNGTDQAWAQSDKNFKVTLDNGSGGTLILGNSTETPSSSGKILFGGTGGNPFALNGNYGGDLGYMTGEFDAGKIYLTGTIESGNSFHDGNGTNISYNAITNITANGLHYLNDNAGAWHSNANFRATNGSINISNSQFQDQSSGQFTFNSQNQTFSSTTFTGNSHTITLHATNNLTLTNTSFNDSNASLTLEADKGSLRDTDNQTSQITAKNLQVIANQASFSNTIFNAQNSSFKTNQLTLTNDTFNNGSYSFAPENNGNHTTTFGGTTTINTSSSPFANLGGSISLNNGAIFNLNNILSSLQIGTTYNILGGSGANILYKNDTQYASALWQLIKINGTTINSETEISDNNDTQVWNVVFNIDGMPIKIQETFASSGLSFKVISQAKDIWSDVYHMTTNCAYNALASPPGCYGYQNGTHNILKHFNKYGFEAYNESVGADGIAYINPLNSQSHDGYNASTHTLQTYTKIGNGGTYNVGEMQNGKWVNNGTLILGNNTSQPVKGGKIEFGKVDSVGYITDVFNAGHIFLTNSIEVGDSSLSGAGATLTFNANNNITADGLTYHQDATAIPPFGSAISQHSYGNFIAQQSFSALNSSFEDDTSGSLDFTGKNSINFTSSTVIGSKSSITLNSANTTLNNSAFFVGNGTTLTFGNVITTNSHSSYSSSTNTINNSTINLNQNSSLYLHGSTTLDNTTSFLNLSSQATFYDSASLSGSTTINLDHNSKITMNSTTTLNDNANFNLINSAQANFQGNSTFNNNSGITLASGAKAMFTHTTTSTQDITTFNNNSFLNVNGSFTDFIPSTNSNRTISGGGSSSNQNYSAQFNDLVFNNYASMLLNSADIQSSQTTFANSVNINMQNSLFNASTLNLNGGNFYMQNSQIKAGAVSVGSSDSVNINSGVNSLNRSLITSSSFNLNGTLNLDGLLLGPTTTGTTKSTANGQPLISVGGGTFSLNGILNISNIDLSAHLSSQTNHTSSSATYDIVNANNITGMSGANGYQKIEYYGIKINNATYSDSNSDSNKTQSWSFTNPLNGAQTITEKIQSGKLTITISNSNHFVATDYHNIAPELFFYKQSAQNLPSTNSASADISSYDYSSDESGTFFLDGNLKGVYYPKSSTTGTTPVIPGTYNAQGQPLQGLYISNNGLFNETTLNNLVDIVRSIWPHLKTLLPKILQDLSDPSNIIQDLENSGIKLTSQQSKELLSFIDGLSSNINQTFNNGTLVVGSPQAGQTGSSSVVWFGGNGYTTACTAAQTEKGCQDLRGTYLGQLLGSTSAALGYIEANFKAKDIYITGTVGSGNAWGIGGSADVTFNSATNLTLNQATIDAEGTDQIFNMLGEGGIQKILGQKGLAQMLGNYIYDLANGSSINLNPISSIPSSIQPLAKDLGRQIGKIKLSDVLSASDVSALLNMPGMDNVIKNILSTKTVSSVLGSRGLISSLNQSEQNKIYGAIDKELHFSGGKVIASIANGVYGNHTLLSLINSLSPMTGKNVDNILNMPSTTSGQSQIKSLLEQTTFEQVFQELLSNQNLLNKTIAWLGPQILDEMLKTAIDDLLNPTNQLTAAEKNILDNILNNVFSSEKKAVQQMENSNPDVKHVIDGLMQAKGLGEVYSKGLQSILSNKLQGQLKSMGLGSLLAPKALGNFWQKGYFNFLANDDILVNNSTFSNASGGTLSFIAGKSIIFAGQNTINFSNNQGTLEFLSNDVSNIDLTTLNATDGLTIDAPFNNLYVQKGNITLNQYESFSVQAGNFDFLGTVQADGAVDLSGVTGLAVLGTLNLTEDSTLKANNLTTISAFNNQSQNLLNISGNFNSYGTFSTQGAGINIGGGFNSIGALTFNVAGATVKTTGPSSDSSSSSTSSSTSSSNSNSSGGSSSSSSTSDSTSSSAPTSSTTATALTLASTTVSTTSTTNSSSDTSNSSSSNSSSSNSVSNAIPVSPTPSTQIPLIQVGGLVNLNLGGSAIISFNTEAQTNSTGTTGTTQGTANTGSTGSSTNSSSTSTSQTTSSSSTSSTQTTSLSSNTSLATTSQTPTTSSGASPDSSNPTASPITPSNGAYTLIQTNSWIHYNPTSFNPNNWRQYLELYTSLKINGTAFQLNAQGTGLTYNGQAVNISQRGLLVNYQGTNGQEVSASIDYNKMQIGIGQSLHVIAPTITQYITQIQGQSVVNALENAGGPGVMNWFGKLLIETKNTPLFAPYYLEQHSLSDLLKIVKDIQNATDWMGASGLKATSSKLLQISVHTKQMSRLAKLSNFASNDALPDFHDFLVSLKGKKFASAVPNAMDIITAYSQRDKLKNNLWITGVGGASFVAGGTGTLYGLNVGYDRFIKGVIVGGYMAYGYSGFYGNINSANSNNVNVGFYSRAFIKGRNEITGSINETYGYNKTYIDATNPILTPLNQQYHYGTWTTNVGANYGYDFFFKNKHVILKPQIGLTYYYIGLSGLQGKMNDPIYNEFRANADPAHKSVLTINLALESRHYFRKNSYYYVIAGLGRDLFVHSMGDKIVRFIGNDMLSYRYGGMYNTFASLTTGGEVRLFRSFYVNAGIGARFGLDYQDINITGNVGMRYAF

SEQ ID NO: 3 - protein, 14 aa, Artificial Sequence
  antigen peptide No.11(SNTW101, HS1, HS5)
EKKAVQQMENSNPD

SEQ ID NO: 4 - protein, 14 aa, Artificial Sequence
  antigen peptide No.11(TKY)
EKKAVEQMENSNPD

SEQ ID NO: 5 - protein, 14 aa, Artificial Sequence
  antigen peptide No.11 (SH8)
EKDAVTSLKNSNSG

SEQ ID NO: 6 - protein, 14 aa, Artificial Sequence
  antigen peptide No.11 (SH10)
EKDAVTSLENSNSG

SEQ ID NO: 7 - protein, 14 aa, Artificial Sequence
  antigen peptide No.19 (SNTW101, HS1, HS5, SH8)
NQGTLEFLSNDVSN

SEQ ID NO: 8 - protein, 14 aa, Artificial Sequence
  antigen peptide No.19 (TKY, SH10)
NQGTLEFLSNDVST

SEQ ID NO: 9 - protein, 14 aa, Artificial Sequence
  antigen peptide No.33 (SNTW101, HS1, HS5)
LSNKLQGQLKSMGL

SEQ ID NO: 10 - protein, 14 aa, Artificial Sequence
  antigen peptide No.33 (TKY, SH8)
LSNKLQDQLKSMGL

SEQ ID NO: 11 - protein, 14 aa, Artificial Sequence
  antigen peptide No.33 (SH10)
FSDKLQNMLKSLNM

SEQ ID NO: 12 - protein, 14 aa, Artificial Sequence
  antigen peptide No.16
TNGQEVSASIDYNK

SEQ ID NO: 13 - protein, 14 aa, Artificial Sequence
  antigen peptide No.23
AKLSNFASNDALPD

SEQ ID NO: 14 - protein, 14 aa, Artificial Sequence
  antigen peptide No.10
PTTSSGASPDSSNP

SEQ ID NO: 15 - protein, 14 aa, Artificial Sequence
  antigen peptide No.21
GLGRDLFVHSMGDK

SEQ ID NO: 16 - protein, 14 aa, Artificial Sequence
  antigen peptide No.15
QIGKIKLSDVLSAS

SEQ ID NO: 17 - protein, 14 aa, Artificial Sequence
  antigen peptide No.34
YGAIDKELHFSGGK

SEQ ID NO: 18 - protein, 14 aa, Artificial Sequence
  antigen peptide No.20
NVDNILNMPSTTSG

SEQ ID NO: 19 - protein, 14 aa, Artificial Sequence
  antigen peptide No.22
GNLKGVYYPKSSTT

SEQ ID NO: 20 - protein, 14 aa, Artificial Sequence
  antigen peptide No.14
ITEKIQSGKLTITI

SEQ ID NO: 21 - protein, 14 aa, Artificial Sequence
  antigen peptide No.26
FHDFLVSLKGKKFA

SEQ ID NO: 22 - protein, 14 aa, Artificial Sequence
  antigen peptide No.31
TTGGEVRLFRSFYV

SEQ ID NO: 23 - protein, 14 aa, Artificial Sequence
  antigen peptide No.35
IGARFGLDYQDINI

SEQ ID NO: 24 - protein, 14 aa, Artificial Sequence
  antigen peptide No.11(mix)
  MISC_FEATURE (3)..(3): Xaa is K or D
  MISC_FEATURE (6)..(6): Xaa is Q,E or T
  MISC_FEATURE (7)..(7): Xaa is Q or S
  MISC_FEATURE (8)..(8): Xaa is M or L
  MISC_FEATURE (9)..(9): Xaa is E or K
  MISC_FEATURE (13)..(13): Xaa is P or S
  MISC_FEATURE (14)..(14): Xaa is D or G
EKXAVXXXXNSNXX

SEQ ID NO: 25 - protein, 14 aa, Artificial Sequence
  antigen peptide No.19 (mix)
  MISC_FEATURE (14)..(14): Xaa is N or T
NQGTLEFLSNDVSX

SEQ ID NO: 26 - protein, 14 aa, Artificial Sequence
  antigen peptide No.33 (mix)
  MISC_FEATURE (1)..(1): Xaa is L or F
  MISC_FEATURE (3)..(3): Xaa is N or D
  MISC_FEATURE (7)..(7): Xaa is G, D or N
  MISC_FEATURE (8)..(8): Xaa is Q or M
  MISC_FEATURE (12)..(12): Xaa is M or L
  MISC_FEATURE (13)..(13): Xaa is G or N
  MISC_FEATURE (14)..(14): Xaa is L or M
XSXKLQXXLKSXXX

SEQ ID NO: 27 - DNA, 9009 bp, Helicobacter suis
  CDS (1)..(9009)
atgaaaaagtttagttctctcacattgaaatttggccacgctctagcacggcgcattaaagctaaccaagctaggcgtgcgggtacttatgtctttaaaaaacaactcccccaacccaagcgttctgagttgaaacctaaattaatcaagcaggggacttttattttaggtgtaatgagccaaccgcttttagcgtggtatccttcatggattagtggcacacacacactcaacacaacaaatattaagcagtatatgcaaaataaccatagaagccaaaatttgttatggactggtggctctaatgtcttttatgaagctagtaatggaagttatttttgtactaactggaattgtaacggctcggttacattaattggtaatggctctaccgcttacacgcttagcaatctcaattacgagggcggtagccttaatttacaaattaatggcaatggtacacaaggcacgcttaatattagcaatgtcaacatggatagctataaaggaaggcaatttacagatacatggaatgcccaaagtgttagtatcagcggaaatctacaagtagggcaaaatcatatcactatcaacgcaaaccacggcactacagcgaacaacgctaccattaacgcggatcaaggcaacgggattttaaatatcaatgataaaacaagcgagagtttcacaaacaccacctttaaaggtacggggcagatcaatttacagagcaacaacatcacctttaagaatgttacttttaatgatagcaacactggttcacatgttacggacaacggcactcttacccttgagggcactgagacctttgcacaaaactcgccccttatcaatctaggcgccaatgttactatccaagccaacaccatttttaacattacagaagatcttacaaagaccaccaacggagcctataatcttgatacccttgtaagtacaagtggtaataaaagtatcaacgatagtagctatgcaagccatttgtgggatcttatccgctatcaaggccaaacaggtagcctttttaacgggcaactcagtaatggcactactttaagtaatcctagctctggcaatgggatttactatgtgaaatatacttttggcaatggtgattgggatattcttaaagaagattttgaaaataactctctatcggcgcaacttgaggcttacgctaaaaatcagggtgatatttggagtacaatccacaacattaattctacttggaattttaatgtcggagagggtagtgcctatatcaaccccgggaatggaacagatcaagcttgggcacaaagagataaaaattttaaagttactttagataatgggagtggtgggacgcttattcttggtaatagcacagaaacccccagttcaagcggtaaaattctctttggaggcacagggggtaatccttttgctttaaatggcaattacggcggagatcttggctatatgacaggggaatttgacgcgggtaaaatttatcttaccggtaccatagaaagcggaaactcttttcacgatggtaacggcactaacattagctacaacgctattacaaacattacagctaatggtttgcactatctcaacgataacgccggagtatggcatagcaacgctaattttagagctaccaatggcagtatcaacatctcaaactcccaattccaagatcaaagtagcgggcaatttacctttaactctcaaaatcaaacttttagtagcacaacttttactggcaatagtcatacgatcactctacacgctaccaacaacctaaccctcaccaacacaagctttaatgattcaaatgctagcttgacactagaggcagataagggtagtttacaagataccgataaccaaactagccaaatcacagctaaaaacctccaagtaattgctaatcaagctagttttagtaacacgatctttaatgcacaaaacagctcttttaaaactaaccaattgaccctgacaaatgatacatttaataatggtagctatagctttgcacctgaaaacaacggcaaccacacaactacatttagcggtaccaccacgattaatactagcagtagtccttttgctaatttaggtggggcgatcaagtttaatagtggcgctacctttaacctgaataatattttaagttctttgcaaattggtacaacttacagcatcttaggcggaagtggggccaacattaattatagtagtgatacgcagtatgccaacaatctttggaatttaatccgtattagcggggctagtatcagctcagaaacagaaatgagtgatagcaacggcacgcaggtttgggatgtggtctttggtataaatggcatgcccattaagatacaagaaacctttgctagtagtggacttagcttaaaagttattagccaagctaaagatatttggtcagatgtgtataacatgactactaattgttcatacaatgccttagccagcccaccgggctgttatggttatcaaaatggtacatacaacatatggaagcactttaataagtacggctttgaagcttacaatgaaagtgtaggcgcagacgggattgcctatatcaaccctttaaactcacaaagccacgatggctataacgcctccacccacacactccaaacttatacaaaacttggcaatggcggtacttataatgttggagaaaaaaaaaatagccaatgggttaacgatggcactttaattttaggtaacaataccctaaaagccgctacagggggtaaaatagaatttggcgcagtgggctcagtaggctatatcaccgatgtttttaatgccggtaatatttttctaactaatactatagaagttggagatagctcgctaagcggggcaggggcgactctaacttttaatgctaataacaatattaccgccgatggcctcacctaccaccaagatgctaccgctatccctccctttggttctactatcagccagcatagccacggtaacttcatcgcacaacaaagctttagcgcgctcaattcctcttttgaagatgatactagcggtagtttagactttaccggtaaaaacagcattaagtttacaagcagcaccgttataggtagtaaaagtagtattacccttaactccgcaaataccaccctaaacaactctgccttttttgtgggcaatggtacgactcttatttttggtaatgtgatcacaactaatagtcatagtagctattcatctagcacaaacaccattaataactctacaatcaacctcaaccaaaactctatcttgtatttgcatggcagtaccaccctagataacaccacttcctttttaaacttaggtagtcaagcaactttttatgattcagctagcctaagtggtagcactacaattaatcttgatcacaatagcaagattataatgaatagcaccacgacgctaaatgataacgctaattttaatttaattaactccgctcaagctaactttcaaggcaatagtacttttaataatagctctgggatcactcttgctagtggtgctaaggctatgtttacacacactacaacaagcacccaagatattacaacttttaataataactcgtttttaaatgtaaacgggagttttacagattttataccaaatacaagtagcgggagcattagcggggggggatcaagtagtaatcaaaactatagcgcgcagtttaataatctagtgtttaacaactatgcctccatgctcttaaatagcgcagatattcaaagtagccaaaccacctttgctaattctgtgaatatcaatatgcaaaatagcctttttaatgcgggtacgcttaatttaaatggcggtaatttttacatgcaaaatagccagattaaagcgggagctgtaaatgtaggatcttctgattctgttaatatcaattctggggtcaattctcttaatcgctcgcttattactagctcaagctttaatttaaacggtactttaaatttagatggtcttttacttgaacccacaactagtacaggtgcaagctcaagcgcagctaacagccaacctcttatttctgtttctaataatagctctagtagcacgggaggggggacatttgatttaagtggtattcttaatatctctaatattgatctctccgcgcccttttctagccaaactaataatacaagttcctccgccacttataatattgtaaatgctagtaagattacaggtatgagtggggctaatggctatcaaaaaattgagtactacggcattaaaattaataacgctacctattctgattcaaataaaacccaaagctggagttttacaaaccctttaaatggcgcacaaactattactgaaaaaatacaaaatggtaaactcactatcaccattagtaatagcaatcattttgtagctacagattaccacaacatcgcccccgaactctttttttacaaacaatccgcacaaaacttgcctagtacaaactcagcaagtgcagatataagcagttatgattattccagcgatgaaagcgggactttctttttagacggtaatttaaaaggggtgtatgatccaaaggctaatacttcaagtagtaaaacttcctcaacaccggttattccgggcacttataatgctcaaggccagcccttacaaggtctttatatttctaataacggcctgtttaatgaaaccacccttaataacttagtaggcattgtccaaagtatttggcctcatttaaaaacccttttgcctcaaattctacaagatttaagcgatccaagtaatatcattcaagacttagaaaactcagggattaaattaactagccagcaaagcaaggaacttttaagttttatagacgggctatcaagcaatatcaaccaaacttttaataatggtacgcttgtagtgggttcgcctcaggcaggacaaaccggaagtagtagcgtggtgtggtttggtggcaatggttatactacggcttgtacggctgcacaaactaaaaaaggatgtcaggatttaagaggcacttatttaggtgagcttctaggctcaacttctgctgcactaggctatattgaggctaattttaaggctaaagatatttatatcaccggcaccgtaggcagtggggatgcttgggggataggtgggagtgcagatgtaacttttaatagcgcgactaatttaacccttaatcaggccacgattgatgcagaaggcacggatcaaatttttaatatgctaggtgagggcgggatacaaaaaatcttaggccaaaaagggctagcccagatgttaggtaattatatctacgatctagccaatggctctagtattaatcttaatcccactagtagcattccaagctctatcaagcctctagctaaagatttaggcgggcagattggtaaaatcaaacttagcgatgtattgaatgcctcagatgtgagtgctcttttaaacatgcccggtatggataatgtgattaaaaatattttaagtactaagactgtgagttctgtattaggcggtggagggcttatttctagtttggatcaggctgagcaaaataaaatctataatgctattgataaagagttacatttctcagggggaaaagccattgcctccattgccaacggggtttatggtaagcacacccttttaagtctcattaactctttatcccctatgacgggtaaagatgtggataatattttaaacatgccaaatactagtagcggccaaagtcaggtaaaaaattttttaaacaagaccacttttgggcaaatttttgaagaactattatctaatcaaaacctactcaataaaaccattgcttggttaggcccacaaattttagatgaaatgcttaaaactgctattgatgatttactcaaccctacaaatcagcttactgccgctgaaaaaaatattcttgataacattcttaacaatgtctttagctcagaaaaaaaggcagtccaacaaatggaaaattctaaccctgatgtcaaacacgttattgatggcttaatgcaggctaaaggtctaggggaggtttatagcaagggccttcagagtattttatctaataaactgcaaggtcaattaaaaagcatgggcttaggttctttgctcgcgcctaaagctctaggtaatttctggcagaagggttattttaacttcctagccaatgatgatattttagtgaacaatagcacttttagtaatgcttcaggcgggactttaagttttatagccggtaagtctattatttttgcagggcaaaatacaattaattttagtaataatcaaggtacactagaatttttaagtaatgatgtgtctaatattgatctgactactcttaatgctaccgatggcctaaccattgatgccccctttaataatctttatgtccagaaaggcaatatcacccttgcttcgtatgaagggctcacagtacgcgcaaataattttgactttttaggcacggtgcaagcagatggggctgtggatttatccggtgtaaccggtttagctgttttaggtactcttaatcttactgaggatagcacccttaaggctaataatttaaccacaataagcgcctttaacaaccaatcccaaaacctgcttaatatcagcgggaattttaactcctatggcacatttagtacacaaggggctggggtcaatatcgggggcgggtttaatagtacaggggctttaacttttaatgtagcgggtgcaacgattaaaaccaccggccctagttcagataattcaacctcaagctctagcgcctctacaagttctagtaatgctaatagctctggaggttctagctcctcccctagcacttctacaccctctactaactctagcgcctctactagttctaaaacagctacagctttaactctagctagcatcaccgtgtctacaagttctacaacttctagtaccgctagctctagcacttctacaagttcctctaacacctcctccagtagcgataatagcccaagctctaatagtgggtctaatgtaaatccggttagcccaacgcctagtacgcaaattccgctaattcaagtgggtggacttgtgaatttaaatttaggtggtagtgcgattattagctttaatacagaggcacaaacaaataatgcaggatcaagcgcgggttctagttctacaagtacaccacaaacaacaactagcagttctactagttctactcaaacagcaagtacagcacaaagtaccacattaagcctaagctctaatactagtctagctactaccagccaaacgcctacaacttctagcggggcttctccggatagttcaaaccctacagcctctcctataaccccttcaaatggcgcttatacgctgattcaaactaatagctggattcattacaaccccacctcttttaatcctaataactggagacaatatttagagctctacactagccttaaaatcaacgggacagctttccagcttaacgctcaaggtacaggacttacttataatggtcaagcagtcaatatctcacaacgaggcttgcttgtcaattatcaaggcacaaatggccaagaagtgagcgcctctattgattataataagatgcaaataggcataggccaaagcttgcatgttatcgcgcccactattacacaatacatcacccaaatacaagggcagtctgtggttaatgcgctagaaaatgcaggcgggccgggtgtgatgaattggtttggtaagcttttaattgagacaaaaaacaccccgctttttgcgccctactatcttgaacaacactccttaagcgatctacttaaaattgtaaaagatattcaaaatgcgactgattggatgggcgcttctggtttaaaagccactagctctaaattgctacaaatcagtgtacacaccaaacagatgagccgtttagctaagctttcaaattttgcttctaatgatgcattaccagattttcatgattttttagtcagtcttaaaggtaaaaaatttgctagcgcggtgcctaatgctatggatattatcaccgcctactcccaaagagataaattaaaaaacaacctatgggtaaccggtgtgggaggagcaagttttgtagcaggaggcacaggtacgctttatgggctgaatgtgggttatgatcgctttataaagggcgtgattgtggggggttatatggcttatggttatagtggcttttatggcaatatcaatagcgctaattctaataatgtcaatgtgggattttatagccgcgcctttatcaagggtagaaacgagattacaggaagtattaatgaaacctatggctataacaaaacctacattgatgcaactaatcccattctcacccctcttaaccagcaataccactatggcacttggactaccaatgtcggcgctaactatggctatgacttcttttttaaaaacaaacatgttattctcaaaccccaaattggcctcacttattattatatcggcctctcaggcttgcaaggcaagatgaatgatccgatttataacgagtttagagccaatgccgatccagcgcataaatctattttgacgatcaatttagccctagagagtcgccactattttaggaaaaactcctattactatgtcattgccggcttagggcgggatttattcgtccattctatgggtgataaaatggtacgctttattggtaatgatatgttaagttatcgttatgggggaatgtataatacctttgctagcctcaccacagggggtgaggttcgcttatttcgatccttttatgtcaatgctggtattggagcgcgctttggtttggattatcaagatattaatattacaggcaatgtcgggatgcgctatgctttttag

SEQ ID NO: 28 - protein, 3002 aa, Helicobacter suis
MKKFSSLTLKFGHALARRIKANQARRAGTYVFKKQLPQPKRSELKPKLIKQGTFILGVMSQPLLAWYPSWISGTHTLNTTNIKQYMQNNHRSQNLLWTGGSNVFYEASNGSYFCTNWNCNGSVTLIGNGSTAYTLSNLNYEGGSLNLQINGNGTQGTLNISNVNMDSYKGRQFTDTWNAQSVSISGNLQVGQNHITINANHGTTANNATINADQGNGILNINDKTSESFTNTTFKGTGQINLQSNNITFKNVTFNDSNTGSHVTDNGTLTLEGTETFAQNSPLINLGANVTIQANTIFNITEDLTKTTNGAYNLDTLVSTSGNKSINDSSYASHLWDLIRYQGQTGSLFNGQLSNGTTLSNPSSGNGIYYVKYTFGNGDWDILKEDFENNSLSAQLEAYAKNQGDIWSTIHNINSTWNFNVGEGSAYINPGNGTDQAWAQRDKNFKVTLDNGSGGTLILGNSTETPSSSGKILFGGTGGNPFALNGNYGGDLGYMTGEFDAGKIYLTGTIESGNSFHDGNGTNISYNAITNITANGLHYLNDNAGVWHSNANFRATNGSINISNSQFQDQSSGQFTFNSQNQTFSSTTFTGNSHTITLHATNNLTLTNTSFNDSNASLTLEADKGSLQDTDNQTSQITAKNLQVIANQASFSNTIFNAQNSSFKTNQLTLTNDTFNNGSYSFAPENNGNHTTTFSGTTTINTSSSPFANLGGAIKFNSGATFNLNNILSSLQIGTTYSILGGSGANINYSSDTQYANNLWNLIRISGASISSETEMSDSNGTQVWDVVFGINGMPIKIQETFASSGLSLKVISQAKDIWSDVYNMTTNCSYNALASPPGCYGYQNGTYNIWKHFNKYGFEAYNESVGADGIAYINPLNSQSHDGYNASTHTLQTYTKLGNGGTYNVGEKKNSQWVNDGTLILGNNTLKAATGGKIEFGAVGSVGYITDVFNAGNIFLTNTIEVGDSSLSGAGATLTFNANNNITADGLTYHQDATAIPPFGSTISQHSHGNFIAQQSFSALNSSFEDDTSGSLDFTGKNSIKFTSSTVIGSKSSITLNSANTTLNNSAFFVGNGTTLIFGNVITTNSHSSYSSSTNTINNSTINLNQNSILYLHGSTTLDNTTSFLNLGSQATFYDSASLSGSTTINLDHNSKIIMNSTTTLNDNANFNLINSAQANFQGNSTFNNSSGITLASGAKAMFTHTTTSTQDITTFNNNSFLNVNGSFTDFIPNTSSGSISGGGSSSNQNYSAQFNNLVFNNYASMLLNSADIQSSQTTFANSVNINMQNSLFNAGTLNLNGGNFYMQNSQIKAGAVNVGSSDSVNINSGVNSLNRSLITSSSFNLNGTLNLDGLLLEPTTSTGASSSAANSQPLISVSNNSSSSTGGGTFDLSGILNISNIDLSAPFSSQTNNTSSSATYNIVNASKITGMSGANGYQKIEYYGIKINNATYSDSNKTQSWSFTNPLNGAQTITEKIQNGKLTITISNSNHFVATDYHNIAPELFFYKQSAQNLPSTNSASADISSYDYSSDESGTFFLDGNLKGVYDPKANTSSSKTSSTPVIPGTYNAQGQPLQGLYISNNGLFNETTLNNLVGIVQSIWPHLKTLLPQILQDLSDPSNIIQDLENSGIKLTSQQSKELLSFIDGLSSNINQTFNNGTLVVGSPQAGQTGSSSVVWFGGNGYTTACTAAQTKKGCQDLRGTYLGELLGSTSAALGYIEANFKAKDIYITGTVGSGDAWGIGGSADVTFNSATNLTLNQATIDAEGTDQIFNMLGEGGIQKILGQKGLAQMLGNYIYDLANGSSINLNPTSSIPSSIKPLAKDLGGQIGKIKLSDVLNASDVSALLNMPGMDNVIKNILSTKTVSSVLGGGGLISSLDQAEQNKIYNAIDKELHFSGGKAIASIANGVYGKHTLLSLINSLSPMTGKDVDNILNMPNTSSGQSQVKNFLNKTTFGQIFEELLSNQNLLNKTIAWLGPQILDEMLKTAIDDLLNPTNQLTAAEKNILDNILNNVFSSEKKAVQQMENSNPDVKHVIDGLMQAKGLGEVYSKGLQSILSNKLQGQLKSMGLGSLLAPKALGNFWQKGYFNFLANDDILVNNSTFSNASGGTLSFIAGKSIIFAGQNTINFSNNQGTLEFLSNDVSNIDLTTLNATDGLTIDAPFNNLYVQKGNITLASYEGLTVRANNFDFLGTVQADGAVDLSGVTGLAVLGTLNLTEDSTLKANNLTTISAFNNQSQNLLNISGNFNSYGTFSTQGAGVNIGGGFNSTGALTFNVAGATIKTTGPSSDNSTSSSSASTSSSNANSSGGSSSSPSTSTPSTNSSASTSSKTATALTLASITVSTSSTTSSTASSSTSTSSSNTSSSSDNSPSSNSGSNVNPVSPTPSTQIPLIQVGGLVNLNLGGSAIISFNTEAQTNNAGSSAGSSSTSTPQTTTSSSTSSTQTASTAQSTTLSLSSNTSLATTSQTPTTSSGASPDSSNPTASPITPSNGAYTLIQTNSWIHYNPTSFNPNNWRQYLELYTSLKINGTAFQLNAQGTGLTYNGQAVNISQRGLLVNYQGTNGQEVSASIDYNKMQIGIGQSLHVIAPTITQYITQIQGQSVVNALENAGGPGVMNWFGKLLIETKNTPLFAPYYLEQHSLSDLLKIVKDIQNATDWMGASGLKATSSKLLQISVHTKQMSRLAKLSNFASNDALPDFHDFLVSLKGKKFASAVPNAMDIITAYSQRDKLKNNLWVTGVGGASFVAGGTGTLYGLNVGYDRFIKGVIVGGYMAYGYSGFYGNINSANSNNVNVGFYSRAFIKGRNEITGSINETYGYNKTYIDATNPILTPLNQQYHYGTWTTNVGANYGYDFFFKNKHVILKPQIGLTYYYIGLSGLQGKMNDPIYNEFRANADPAHKSILTINLALESRHYFRKNSYYYVIAGLGRDLFVHSMGDKMVRFIGNDMLSYRYGGMYNTFASLTTGGEVRLFRSFYVNAGIGARFGLDYQDINITGNVGMRYAF

SEQ ID NO: 29 - DNA, 4593 bp, Helicobacter suis
  CDS (1)..(4593)
attactgaaaaaatacaaaatggtaaactcactatcaccattagtaatagcaatcattttgtagctacagattaccacaacatcgcccccgaactctttttttacaaacaatccgcacaaaacttgcctagtacagactcagtaagtgcagatataagcagttatgattattccagcgatgaaagcgggactttctttttagatggcaatttaaaaggggtgtattatccaaaatctagcacaacaggcactacctcaaccccagttattccgggtacttataacgctcaaggccagcccttacaaggtctttatatttctaataacggcctgtttaatgaaaccacccttaataacttagtaggcattgtccaaagtatttggcctcatttaaaaacccttttgcctcaaattctacaagatttaagcgatccaagtaatatcattcaagacttagaaaactcaaggattaaattaactagccagcaaagcaaggaacttttaagttttatagacgggctatcaagcaatatcaaccaaacttttaataatggtacgcttgtagtgggttcgcctcaggcaggacaaaccggaagtagtagcgtggtgtggtttggtggcaatggttatactacggcttgtacggctgcacaaactgaaaaagggtgtcaggatttaagaggcacttatttaggtcagcttttaggatcaacttctgctgcactaggctatattgaggctaattttaaggctaaagatatttatatcaccggcaccgtaggcagtgggaatgcttgggggataggtgggagtgcagatgtaacttttaatagcgcgactaatttaaccctcaatcaagccacgattgatgcggaaggcacggatcaaatttttaatatgctaggtgagggcgggatacaaaaaatcttaggccaaaaggggctagcccagatgttaggtaattatatctacgatctagccaatggctctagtattaatcttaatcccactagtagcattccaagctctatccagcctctagctaaagatttaggcgggcagattggtaaaatcaaacttagcgatgtattgaatgcctcagatgtgagtgctcttttaaacatgcccggtatggataatgtgattaaaaatattttaagtactaagactgtgagctctgtgctaggcagtggaggacttatttctagtttgaatcaagccgagcaaaataaaatctatgatgctattgataaagagttacatttctcagggggaaaagccattgcctccattgccaacggggtttatggtaagcacacccttttaagtctcattaactctttatcccctatgacgggtaaagatgtggataatattttaaacatgccaaatactagtagcggccaaaatcaggtacaaaattttttaaagaacaccacttttgggcaaatttttgaagaactattatctaatcaaaacctactcaataaaaccattgcttggttaggcccacaaattttagatgaaatgcttaaaactgctattgatgatttactcaaccctacaaatcagcttactgccgctgaaaaaaatattcttgataacattcttaacaatgtctttagctcagaaaaaaaggcagtccaacaaatggaaaattctaaccctgatgtcaaacacgttattgatggcttaatgcaggctaaaggtctaggggaggtttatagcaagggccttcagagtattttatctaataaactgcaaggtcaattaaaaagcatgggcttaggttctttgctcgcgcctaaagctctaggtaatttctggcagaagggttattttaacttcctagctaatgatgatattttagtgaacaatagcacttttagtaatgcttcaggcgggactttaagttttatagccggtaagtctattatttttgcagggcaaaatacaattaattttagtaataatcaaggtacactagaatttttaagtaatgatgtgtctaatattgatctaactactcttaatgctaccgatggcctaaccattgatgccccctttaataatctttatgtccagaaaggcaatatcacccttaatcaatacgagagttttagcgtgcaggcgggcaattttgactttttaggcacggtgcaagcagacggggctgtggatttatccggtgtaaccggtttagctgttttaggtactcttaatcttactgaggatagcacccttaaggctaataatttaaccacaataagcgcctttaataaccagtctcaaaacttacttaacatcagcgggaattttaactcctatggcacttttagcacgcaaggggctggggtaaatatcgggggcgggtttaatagtacaggggctttaacttttaatgtagcgggtgcaacagtcaaaactacaggccctagctcagatagttcaagttcaagcactagctcctctacaagttctagtaattctaatagctctggaggctctagctcctcccctagcacttctacaccctctactaactctagcgcctctactagttctaaaacagctacagctttaactctagctagcatcaccgtgtctacaagttctacaacttctagtaccgctagctctagcacttctacaagttcctctaacacctcctccagtagcgataatagcccaagctctaatagtgggtctaatgtaaatccggttagcccaacgcctagtacgcaaattccgctaattcaagtgggtggacttgtgaatttaaatttaggtggtagtgcgattattagctttaatacagaggcacaaacaaataatgcaggatcaagcgcgggttctagttctacaagtacaccacaaacaacaactagcagttctactagttctactcaaacagcaagtacagcacaaagtaccacattaagcctaagctctaatactagtctagctactaccagccaaacgcctacaacttctagcggggcttctccggatagttcaaaccctacagcctctcctataaccccttcaaatggcgcttatacgctgattcaaactaatagctggattcattacaaccccacctcttttaatcctaataactggagacaatatttagagctctacactagccttaaaatcaacgggacagctttccagcttaacgctcaaggtacaggacttacttataatggtcaagcagtcaatatctcacaacgaggcttgcttgtcaattatcaaggcacaaatggccaagaagtgagcgcctctattgattataataagatgcaaataggcataggccaaagcttgcatgttatcgcgcccactattacacaatacatcacccaaatacaagggcagtctgtggttaatgcgctagaaaatgcaggcgggccgggtgtgatgaattggtttggtaagcttttaattgagacaaaaaacaccccgctttttgcgccctactatcttgaacaacactccttaagcgatctacttaaaattgtaaaagatattcaaaatgcgactgattggatgggcgcttctggtttaaaagccactagctctaaattgctacaaatcagtgtacacaccaaacagatgagccgtttagctaagctttcaaattttgcttctaatgatgcattaccagattttcatgattttttagtcagtcttaaaggtaaaaaatttgctagcgcggtgcctaatgctatggatattatcaccgcctactcccaaagagataaattaaaaaacaacctatgggtaaccggtgtgggaggagcaagttttgtagcaggaggcacaggtacgctttatgggctgaatgtgggttatgatcgctttataaagggcatgattgtggggggttatatggcttatggttatagtggcttttatggcaatatcaatagcgctaattctaataatgtcaatgtgggattttatagccgcgcctttatcaagggtagaaacgagattacaggaagtattaatgaaacctatggctataacaaaacctacattgatgcaactaatcccattctcacccctcttaaccagcaataccactatggcacttggactaccaatgtcggtgctaactatggctatgacttcttttttaaaaacaaacatgttattctcaaaccccaaattggcctcacttattattatatcggcctctcaggcttgcaaggcaagatgaatgatccgatttataacgagtttagagccaatgccgatccagcgcataaatctgttttgacgatcaatttagccctagagagtcgccactattttaggaaaaactcctattactatgtcattgctggattgggtcgggatttattcgtccattctatgggcgataaaatggtacgctttattggtaatgatatgttaagttatagctatgggggaatgtataatacctttgctagcctcaccacagggggtgaggttcgcttatttcgatccttttatgtcaatgctggtattggagcgcgctttggtttggattatcaagatattaatattacaggcaatgtcgggatgcgctatgctttttag

SEQ ID NO: 30 - protein, 1530 aa, Helicobacter suis
ITEKIQNGKLTITISNSNHFVATDYHNIAPELFFYKQSAQNLPSTDSVSADISSYDYSSDESGTFFLDGNLKGVYYPKSSTTGTTSTPVIPGTYNAQGQPLQGLYISNNGLFNETTLNNLVGIVQSIWPHLKTLLPQILQDLSDPSNIIQDLENSRIKLTSQQSKELLSFIDGLSSNINQTFNNGTLVVGSPQAGQTGSSSVVWFGGNGYTTACTAAQTEKGCQDLRGTYLGQLLGSTSAALGYIEANFKAKDIYITGTVGSGNAWGIGGSADVTFNSATNLTLNQATIDAEGTDQIFNMLGEGGIQKILGQKGLAQMLGNYIYDLANGSSINLNPTSSIPSSIQPLAKDLGGQIGKIKLSDVLNASDVSALLNMPGMDNVIKNILSTKTVSSVLGSGGLISSLNQAEQNKIYDAIDKELHFSGGKAIASIANGVYGKHTLLSLINSLSPMTGKDVDNILNMPNTSSGQNQVQNFLKNTTFGQIFEELLSNQNLLNKTIAWLGPQILDEMLKTAIDDLLNPTNQLTAAEKNILDNILNNVFSSEKKAVQQMENSNPDVKHVIDGLMQAKGLGEVYSKGLQSILSNKLQGQLKSMGLGSLLAPKALGNFWQKGYFNFLANDDILVNNSTFSNASGGTLSFIAGKSIIFAGQNTINFSNNQGTLEFLSNDVSNIDLTTLNATDGLTIDAPFNNLYVQKGNITLNQYESFSVQAGNFDFLGTVQADGAVDLSGVTGLAVLGTLNLTEDSTLKANNLTTISAFNNQSQNLLNISGNFNSYGTFSTQGAGVNIGGGFNSTGALTFNVAGATVKTTGPSSDSSSSSTSSSTSSSNSNSSGGSSSSPSTSTPSTNSSASTSSKTATALTLASITVSTSSTTSSTASSSTSTSSSNTSSSSDNSPSSNSGSNVNPVSPTPSTQIPLIQVGGLVNLNLGGSAIISFNTEAQTNNAGSSAGSSSTSTPQTTTSSSTSSTQTASTAQSTTLSLSSNTSLATTSQTPTTSSGASPDSSNPTASPITPSNGAYTLIQTNSWIHYNPTSFNPNNWRQYLELYTSLKINGTAFQLNAQGTGLTYNGQAVNISQRGLLVNYQGTNGQEVSASIDYNKMQIGIGQSLHVIAPTITQYITQIQGQSVVNALENAGGPGVMNWFGKLLIETKNTPLFAPYYLEQHSLSDLLKIVKDIQNATDWMGASGLKATSSKLLQISVHTKQMSRLAKLSNFASNDALPDFHDFLVSLKGKKFASAVPNAMDIITAYSQRDKLKNNLWVTGVGGASFVAGGTGTLYGLNVGYDRFIKGMIVGGYMAYGYSGFYGNINSANSNNVNVGFYSRAFIKGRNEITGSINETYGYNKTYIDATNPILTPLNQQYHYGTWTTNVGANYGYDFFFKNKHVILKPQIGLTYYYIGLSGLQGKMNDPIYNEFRANADPAHKSVLTINLALESRHYFRKNSYYYVIAGLGRDLFVHSMGDKMVRFIGNDMLSYSYGGMYNTFASLTTGGEVRLFRSFYVNAGIGARFGLDYQDINITGNVGMRYAF

SEQ ID NO: 31 - DNA, 20 bp, Artificial Sequence
  primer FW1
cttttagcgtggtatccttc

SEQ ID NO: 32 - DNA, 20 bp, Artificial Sequence
  primer FW2
ttacaaagaccaccaacgga

SEQ ID NO: 33 - DNA, 19 bp, Artificial Sequence
  primer FW3
taccatagaaagcggaaac

SEQ ID NO: 34 - DNA, 20 bp, Artificial Sequence
  primer FW4
agctatagctttgcacctga

SEQ ID NO: 35 - DNA, 23 bp, Artificial Sequence
  primer FW5
actaacagcatagaagttgggga

SEQ ID NO: 36 - DNA, 20 bp, Artificial Sequence
  primer FW6
aacaacattacaggcatgag

SEQ ID NO: 37 - DNA, 20 bp, Artificial Sequence
  primer FW7
caaatagtaacaggacaatt

SEQ ID NO: 38 - DNA, 20 bp, Artificial Sequence
  primer FW8
caggatttaagaggcactta

SEQ ID NO: 39 - DNA, 19 bp, Artificial Sequence
  primer FW9
tcttaaccaatctgagcaa

SEQ ID NO: 40 - DNA, 23 bp, Artificial Sequence
  primer FW10
gtcaaacacgttattgatggctt

SEQ ID NO: 41 - DNA, 19 bp, Artificial Sequence
  primer FW11
ggggtttaatagcataggg

SEQ ID NO: 42 - DNA, 20 bp, Artificial Sequence
  primer FW12
tagagctctacaccagtctt

SEQ ID NO: 43 - DNA, 35 bp, Artificial Sequence
  primer FW13
ataaagcccatgaattcttaggcatgcgtgctctg

SEQ ID NO: 44 - DNA, 30 bp, Artificial Sequence
  primer FW14
tgctattgataaagagttacatttctcagg

SEQ ID NO: 45 - DNA, 19 bp, Artificial Sequence
  primer FW14-1
atttctcagggggaaagtc

SEQ ID NO: 46 - DNA, 33 bp, Artificial Sequence
  primer FW15
ggctaataatttaaccacaataagcgcctttaa

SEQ ID NO: 47 - DNA, 19 bp, Artificial Sequence
  primer forward
gatgggcgcttctggttta

SEQ ID NO: 48 - DNA, 25 bp, Artificial Sequence
  primer HSVAFW1
atgaattcttaggcatgcgtgctct

SEQ ID NO: 49 - DNA, 22 bp, Artificial Sequence
  primer HSVAFW2
ttttggaggtgtaggagtagat

SEQ ID NO: 50 - DNA, 20 bp, Artificial Sequence
  primer HSVAFW3
agcgcgcatttaaaacaggt

SEQ ID NO: 51 - DNA, 20 bp, Artificial Sequence
  primer HSVANOFW-2
agaaacgagattacaggaag

SEQ ID NO: 52 - DNA, 20 bp, Artificial Sequence
  primer HSVANOFW-3
aggagcaagttttgtagcag

SEQ ID NO: 53 - DNA, 20 bp, Artificial Sequence
  primer HSVANOFW-5
aaaatgcgactgattggatg

SEQ ID NO: 54 - DNA, 18 bp, Artificial Sequence
  primer FWNO3
ttgaaatttggccacgct

SEQ ID NO: 55 - DNA, 20 bp, Artificial Sequence
  primer RV1
tcacccatagaatggacgaa

SEQ ID NO: 56 - DNA, 22 bp, Artificial Sequence
  primer RV2
ctagcgcattaaccacagactg

SEQ ID NO: 57 - DNA, 19 bp, Artificial Sequence
  primer RV3
tagaagttgtagacacggt

SEQ ID NO: 58 - DNA, 20 bp, Artificial Sequence
  primer RV4
gtgatattgcctttctgaac

SEQ ID NO: 59 - DNA, 18 bp, Artificial Sequence
  primer RV5
gcaagttttgtgcggatt

SEQ ID NO: 60 - DNA, 19 bp, Artificial Sequence
  primer RV7
atgtgatacacatctgacc

SEQ ID NO: 61 - DNA, 20 bp, Artificial Sequence
  primer RV8
agtgccgttaccatcgtgaa

SEQ ID NO: 62 - DNA, 35 bp, Artificial Sequence
  primer RV9
tattcaaggaaagtccctggagaaactccagagac

SEQ ID NO: 63 - DNA, 33 bp, Artificial Sequence
  primer RV10
ttaaaggcgcttattgtggttaaattattagcc

SEQ ID NO: 64 - DNA, 21 bp, Artificial Sequence
  primer RV10-1
attagccttaagggtgctatc

SEQ ID NO: 65 - DNA, 25 bp, Artificial Sequence
  realtime-PCR Reverse
ctggtaatgcatcattagaagcaaa

SEQ ID NO: 66 - DNA, 20 bp, Artificial Sequence
  primer HSAVRV
tacgcgcaaaataggttctt

SEQ ID NO: 67 - DNA, 20 bp, Artificial Sequence
  primer HSAVRV2
tggagaaactccagagacta

SEQ ID NO: 68 - DNA, 21 bp, Artificial Sequence
  primer HSAVRV3
ttgttcgctgtagtgccgtgg

SEQ ID NO: 69 - DNA, 20 bp, Artificial Sequence
  primer RVNO4
gaaggataccacgctaaaag

SEQ ID NO: 70 - DNA, 20 bp, Artificial Sequence
  primer RVNO5
aagctagagttttggttgag

SEQ ID NO: 71 - DNA, 19 bp, Artificial Sequence
  primer RVNO6
ttgctcagattggttaaga

SEQ ID NO: 72 - DNA, 20 bp, Artificial Sequence
  primer HSVANORV-1
atcgaaataagcgaacctca

SEQ ID NO: 73 - DNA, 20 bp, Artificial Sequence
  primer HSVANORV-3
ttgaaagcttagctaaacgg

SEQ ID NO: 74 - DNA, 20 bp, Artificial Sequence
  primer HSVANORV-4
tggtattgctggttaagagg

SEQ ID NO: 75 - DNA, 16 bp, Artificial Sequence
  realtime PCR probe
caaacagatgagccgt

SEQ ID NO: 76 - DNA, 43 bp, Artificial Sequence
  primer HSVASTART
atgaaaaagtttagttctctcacattgaaatttggccacgctc

SEQ ID NO: 77 - DNA, 45 bp, Artificial Sequence
  primer HSVASTOP
ctaaaaagcatagcgcatcccgacattgcctgtaatattaatatc

SEQ ID NO: 78 - protein, 257 aa, Artificial Sequence
  SNTW101 partial peptide
LEQTTFEQVFQELLSNQNLLNKTIAWLGPQILDEMLKTAIDDLLNPTNQLTAAEKNILDNILNNVFSSEKKAVQQMENSNPDVKHVIDGLMQAKGLGEVYSKGLQSILSNKLQGQLKSMGLGSLLAPKALGNFWQKGYFNFLANDDILVNNSTFSNASGGTLSFIAGKSIIFAGQNTINFSNNQGTLEFLSNDVSNIDLTTLNATDGLTIDAPFNNLYVQKGNITLNQYESFSVQAGNFDFLGTVQADGAVDLSGVT

SEQ ID NO: 79 - protein, 257 aa, Artificial Sequence
  HS1 partial peptide
LNKTTFGQIFEELLSNQNLLNKTIAWLGPQILDEMLKTAIDDLLNPTNQLTAAEKNILDNILNNVFSSEKKAVQQMENSNPDVKHVIDGLMQAKGLGEVYSKGLQSILSNKLQGQLKSMGLGSLLAPKALGNFWQKGYFNFLANDDILVNNSTFSNASGGTLSFIAGKSIIFAGQNTINFSNNQGTLEFLSNDVSNIDLTTLNATDGLTIDAPFNNLYVQKGNITLASYEGLTVRANNFDFLGTVQADGAVDLSGVT

SEQ ID NO: 80 - protein, 257 aa, Artificial Sequence
  HS5 partial peptide
LKNTTFGQIFEELLSNQNLLNKTIAWLGPQILDEMLKTAIDDLLNPTNQLTAAEKNILDNILNNVFSSEKKAVQQMENSNPDVKHVIDGLMQAKGLGEVYSKGLQSILSNKLQGQLKSMGLGSLLAPKALGNFWQKGYFNFLANDDILVNNSTFSNASGGTLSFIAGKSIIFAGQNTINFSNNQGTLEFLSNDVSNIDLTTLNATDGLTIDAPFNNLYVQKGNITLNQYESFSVQAGNFDFLGTVQADGAVDLSGVT

SEQ ID NO: 81 - protein, 257 aa, Artificial Sequence
  TKY partial peptide
LNNTTFGQVLEELLSNQNLLNKTIAWLGPQILDEMLKTAIDDLLNPTSQLTAAEKNILDNILNNVFGSEKKAVEQMENSNPDVKQVIDGLMQAKGLGEVYSKGLQSILSNKLQDQLKSMGLGSLLAPKALGNFWQKGYFNFLANDDILVSNSTFDNASGGTLSFIAGKSIIFAGQNTINFSNNQGTLEFLSNDVSTIDLTTLNATDGLTIDAPFNNLYVQKGNITLNQYESFSVQSGNFDFLGTVQADGAVDLSGVT

SEQ ID NO: 82 - protein, 257 aa, Artificial Sequence
  SH8 partial paptide
LKQTNFTQAFQALESNQNLLNKTIAWVGPQILDEMLQTAINDLLNPTQELTKAETDILNNILNNVFKKEKDAVTSLKNSNSGIKTMIEGLINNKGLGEVYSKGLQSILSNKLQDQLKSMGLGSLLAPKALGNFWQKGYFNFLANDDILVSNSTFDNASGGTLSFIAGKSIIFAGQNTINFSNNQGTLEFLSNDVSNIDLTTLNATDGLTIDAPFNNLYVQKGNITLNQYESFSVQSGNFDFLGTVQADGAVDLSGVT

SEQ ID NO: 83 - protein, 257 aa, Artificial Sequence
  SH10 partial peptide
LKQTNFSQAFQALVSNQNLLNKTIAWLGPQILDEMLQTAINDLLNPTQELTKAETDILNNILNNVFKKEKDAVTSLENSNSGIKTMIEGLINNKGLGGVYTKGLQSIFSDKLQNMLKSLNMGSLLEPKALGKFWEKGYFNFLANDNVLVSNSTFKNASGGTLSFIAGKSIIFSGQNTINFSNNQGTLEFLSNDVSTIDLTTLNATDGLTIDAPFNNLYVQKGNITLNQYESFSVQSGNFDFLGTVQADGAVDLSGVT

SEQ ID NO: 84 - DNA, 20 bp, Artificial Sequence
  primer VAC3624F
gagcgagctatggttatgac

SEQ ID NO: 85 - DNA, 21 bp, Artificial Sequence
  primer VAC4041R
cattcctaaattggaagcgaa

SEQ ID NO: 86 - protein, 14 aa, Artificial Sequence
  antigen peptide No.8
KQLPQPKRSELKPK

SEQ ID NO: 87 - protein, 14 aa, Artificial Sequence
  antigen peptide No. 31N
TNIKQYMQNNHRSQ

SEQ ID NO: 88 - protein, 14 aa, Artificial Sequence
  antigen peptide No. 81
TLTLEGTETFAQNS

SEQ ID NO: 89 - protein, 14 aa, Artificial Sequence
  antigen peptide No.63
EAYAKNQGDIWSTI

SEQ ID NO: 90 - protein, 14 aa, Artificial Sequence
  antigen peptide No. 73
VIGSKSSITLNSAN

SEQ ID NO: 91 - protein, 14 aa, Artificial Sequence
  antigen peptide No. 61
ADIQSSQTTFANSV

(19) United States
(12) Patent Application Publication
(10) Pub. No.: US 2021/0292375 A1
(43) Pub. Date: Sep. 23, 2021

(54) METHOD FOR DIAGNOSING INFECTION WITH HELICOBACTER SUIS

(71) Applicant: THE KITASATO INSTITUTE, Tokyo (JP)

(72) Inventors: Hidenori MATSUI, Tokyo (JP); Masahiko NAKAMURA, Tokyo (JP); Somay MURAYAMA, Tokyo (JP); Tetsufumi TAKAHASHI, Tokyo (JP); Emiko RIMBARA, Tokyo (JP); Masato SUZUKI, Tokyo (JP); Keigo SHIBAYAMA, Tokyo (JP)

(21) Appl. No.: 17/057,499

(22) PCT Filed: May 22, 2019
(86) PCT No.: PCT/JP2019/020248

§ 371 Date: Jun. 3, 2021

(30) Foreign Application Priority Data
May 23, 2018 (JP) 2018-098504
May 24, 2018 (JP) 2018-099903

(51) Int. Cl. C07K 14/205; G01N 33/569; A61P 31/04; A61K 39/02; C07K 16/12

(52) U.S. Cl. CPC C07K 14/205; G01N 33/56922; A61K 2039/53; A61K 39/105; C07K 16/121; A61P 31/04

(57) ABSTRACT

Described herein is a newly found outer membrane protein gene specific for Helicobacter suis and a gene product thereof. Also described herein is a method for determining infection of a subject with Helicobacter suis and a method for treatment using an antibody against the gene product.

### TECHNICAL FIELD

The present invention relates to a newly found outer membrane protein gene specific for Helicobacter suis and a gene product (protein) thereof, and to a method and a drug for diagnosing Helicobacter suis infection using an antibody thereagainst.

### BACKGROUND ART

It is currently known that infection with Helicobacter pylori (a microaerophilic gram-negative spiral bacterium parasitic in the human stomach; hereinafter, referred to as “H. pylon”) is involved in chronic gastritis, gastric ulcer, duodenal ulcer, stomach cancer, gastric mucosa-associated lymphoid tissue (MALT) lymphoma, and diffuse large B-cell lymphoma. An isolation culture method, a urea breath test, measurement of H. pylori antibody titers in serum or urine (ELISA and latex agglutination method), and measurement of H. pylori antigens in feces (immunochromatography) are typically adopted as methods for testing for H. pylori infection.

However, as diagnostic techniques and disinfectants for H. pylori have become widely available, Helicobacter heilmannii in a broad sense has come to be recognized as a species of Helicobacter, other than H. pylori, causing severe gastric diseases in humans. The Helicobacter heilmannii in a broad sense (Helicobacter heilmannii sensu lato; hereinafter, referred to as “H. heilmannii”) includes Helicobacter heilmannii in a narrow sense (H. heilmannii sensu stricto), H. suis, H. bizzozeronnii, H. felis, and H. salmonis. Among them, Helicobacter suis (hereinafter, referred to as “H. suis”) is a species of Helicobacter often found in the human stomach. Nonetheless, a rapid diagnosis method has not yet been established therefor (Non Patent Literature 1).

H. pylori infects only primates, which presumably get infection from close relatives only in childhood, whereas H. suis is transmitted through animals such as pigs, cats, and dogs, irrespective of age. H. pylori is 2.5 to 5.0 μm in the whole length, while H. heilmannii is 5 to 10 μm (Non Patent Literature 2). H. heilmannii including H. suis lacks primary pathogenic factors of H. pylori, i.e., a gene of a type IV secretion apparatus-related protein called CagPAI (pathogenicity island), a gene cluster including genes of protein inducing cancer through injection into hosts, and a gene of a protein toxin, called VacA (vacuolating cytotoxin A), exhibiting a wide variety of effects such as development of erosion or ulcer in gastric mucosa, killing of cultured cells by vacuolar degeneration, and induction of apoptosis (Non Patent Literature 3).

It has also been reported that H. suis infection highly frequently causes development of gastric MALT lymphoma composed mainly of accumulated lymphocytes. Infection experiments using mice have shown that the infectivity of H. suis is much stronger than that of H. pylori (Non Patent Literature 4). In addition, it has been reported that H. suis has flagella at both ends, is highly motile, invades a deep part of a cavity of a gland or parietal cells of the stomach, and is resistant to antibiotics (Non Patent Literature 5).

In fact, approximately 60% or more of patients manifesting symptoms of gastritis or gastric diseases despite being negative for H. pylori are considered to be positive for H. suis infection (Non Patent Literature 6). Also, 25 to 50% of MALT lymphoma cases are considered to be caused by H. suis infection (Non Patent Literature 5).

However, H. pylori is urease-positive, while H. suis separated from humans often exhibits negativity in urease tests or urea breath tests despite having the urease gene (H. suis separated from animals is positive in urease tests or urea breath tests). Accordingly, infection with H. suis cannot be diagnosed by urease tests or urea breath tests which are used for H. pylori. Although there is a report on successful in vitro culture of a strain separated from pigs (Patent Literature 1), there is no report on the colony formation of H. suis in an isolation agar medium. A strain separated from humans cannot be cultured in vitro, and H. suis contained in gastric biopsy of human patients still cannot be purely cultured in vitro (Non Patent Literature 7). Hence, diagnosis by an isolation culture method which is used for H. pylori cannot be used for H. suis. Accordingly, H. suis found in humans cannot be detected by usual testing methods for H. pylori because of being poorly culturable and having weak urease activity.

Meanwhile, the outline of the whole genomes (draft genomes) of an H. heilmannii sensu stricto ASB1 strain isolated from a cat with severe gastritis (Non Patent Literature 8), an H. bizzozeronnii CIII-1 strain isolated from human gastric mucosa (Non Patent Literature 9), H. suis H1 and H5 strains isolated from pig gastric mucosa (Non Patent Literature 10), an H. felis CS1 strain (Non Patent Literature 11), and an H. suis SNTW101 strain isolated from a Japanese patient with nodular gastritis (Non Patent Literature 12) have been analyzed and reported. PCR testing targeting H. heilmannii-specific 16S rRNA gene after preparation of DNA from gastric biopsy is currently a general diagnosis method for infection with H. heilmannii including H. suis.

However, existing diagnosis methods for H. heilmannii require tissue testing and fail to discriminate H. suis from other species of H. heilmannii for diagnosis (Patent Literature 2). Thus, neither a method capable of diagnosing H. suis infection exclusively nor a non-invasive method has yet been established (Non Patent Literature 13). Accordingly, the development of a simple and highly sensitive testing method for H. suis has been desired.

The present inventors have previously reported a method for diagnosing H. suis infection, targeting an F2R2 protein and a gene sequence thereof, based on genome analysis information on H. suis isolated from cynomolgus monkeys (Patent Literature 3).

### CITATION LIST

### Patent Literature

Patent Literature 1: Japanese Patent Laid-Open No. 2011-15664
Patent Literature 2: U.S. Patent Publication No. 2006-0078919
Patent Literature 3: Japanese Patent Laid-Open No. 2016-10331

### Non Patent Literature

Non Patent Literature 1: Blaecher C et al., Helicobacter. (2016) 22 (3): e12369
Non Patent Literature 2: Overby A et al., Digestion. (2016) 93 (4): 260-5
Non Patent Literature 3: Vermoote M et al., Vet Res. (2011) 42: 51
Non Patent Literature 4: Nakamura M et al., Infect Immun. (2007) 75 (3): 1214-22
Non Patent Literature 5: Overby A et al., Digestion. (2017) 95 (1): 61-6
Non Patent Literature 6: Masahiko Nakamura et al., Modern Medical Laboratory (2016) 44 (4): 278-84
Non Patent Literature 7: Matsui H et al., Helicobacter. (2014) 19 (4): 260-71
Non Patent Literature 8: Smet A et al., Genome Announcements (2013) 1 (1): e00033-12
Non Patent Literature 9: Schott T et al., Journal of Bacteriology (2011) 193 (17): 4565-6
Non Patent Literature 10: Vermoote M et al., Veterinary Research (2011) 42: 51
Non Patent Literature 11: Arnold I C et al., Genome Biol. Evol. (2011) 3: 302-8
Non Patent Literature 12: Matsui H et al., Genome Announcements (2016) 4 (5): e00934-16
Non Patent Literature 13: Bento-Miranda M et al., World J Gastroenterol (2014) 20 (47): 17779-87

### SUMMARY OF INVENTION

### Technical Problem

An object of the present invention is to provide an H. suis-specific diagnosis method. Another object of the present invention is to provide a simple and rapid testing method including an H. suis-specific diagnosis method that is a non-invasive method.

### Solution to Problem

The present inventors newly found this time that an outer membrane protein (HsvA; named by the present inventor) specifically present exclusively in H. suis and an antibody against this protein are present in the body of a subject infected with H. suis. Further, the present inventors found a diagnosis method using an amino acid sequence that acts as the antigenic site of the HsvA protein. The F2R2 protein lucks structures such as an amino-terminal signal peptide region common in bacterial cell outer membrane proteins, a carboxy-terminal autotransporter (outer membrane transport mechanism of a protein unique to gram-negative bacteria), and a disordered region involved in the exertion of functions, which are present in the HsvA protein targeted by the present invention (see FIG. 1). Thus, subjects infected with H. suis can be expected to have a higher antibody titer against the HsvA protein, a putative outer membrane protein, than that against the F2R2 protein.

The present inventors have conducted various studies on a method capable of specifically detecting H. suis, and consequently found that an antibody against an H. suis-specific outer membrane protein HsvA is present in the blood of a patient infected with H. suis. The present inventors have revealed that H. suis infection can be detected while discriminating from H. pylori infection, by using an amino acid sequence of the antigenic site of the HsvA protein.

The hsvA gene is contained in an H. suis SNTW101 strain (see Matsui H et al., Genome Announcements (2016) 4 (5): e00934-16) separated from a patient with nodular gastritis. Its nucleotide sequence (hsvA gene: SEQ ID NO: 1) largely differs in molecular weight from and has no identity with that of an H. pylori vacuolating cytotoxin protein VacA (Cover T L et al., Nat Rev Microbiol. (2005) 3 (4): 320-32). However, the HsvA protein has an amino-terminal signal peptide region and a carboxy-terminal autotransporter (outer membrane transport mechanism of a protein unique to gram-negative bacteria) β-domain characteristic of an H. pylori outer membrane protein. This protein also has a disordered region involved in the exertion of functions (see FIG. 1). The hsvA gene is also contained in the draft genomes of H. suis HS1 and HS5 strains separated from a pig (Vermoote M et al., Vet Res. (2011) 42: 51). However, the HsvA protein derived from these strains has no identity with the H. pylori VacA protein. Thus, the hsvA gene had been considered not only to lack the functions of H. pylori VacA but to be not expressed in the first place. However, the present inventors have newly found the presence of the HsvA protein and an antibody thereagainst in the body of a subject infected with H. suis and conducted further studies on HsvA. As a result, the present inventors have found for the first time that the HsvA protein is expressed as an outer membrane protein in H. suis.

The present invention is based on these findings. Accordingly, the present invention relates to the following aspects.

(1) An HsvA antigen peptide or an immunologically equivalent mutant thereof.
(2) The antigen peptide or the immunologically equivalent mutant thereof of (1), derived from H. suis.
(3) The antigen peptide or the immunologically equivalent mutant thereof of (1), wherein the HsvA antigen peptide has a sequence consisting of 5 to 50 amino acids contained in any one of the amino acid sequences set forth in SEQ ID NO: 2, SEQ ID NO: 28, SEQ ID NO: 30, and SEQ ID NOs: 78 to 83.
(4) The peptide or the immunologically equivalent mutant thereof of (3), wherein the HsvA antigen peptide has a sequence consisting of 5 to 50 amino acids contained in any one of the amino acid sequences set forth in SEQ ID NOs: 78 to 83.
(5) The peptide or the immunologically equivalent mutant thereof of (1), wherein the HsvA antigen peptide is a peptide having any one amino acid sequence selected from the group of the following:

(peptide No. 11: SEQ ID NO: 24)
EKX1AVX2X3X4X5NSNX6X7;
(peptide No. 19: SEQ ID NO: 25)
NQGTLEFLSNDVSX8;
(peptide No. 33: SEQ ID NO: 26)
X9SX10KLQX11X12LKSX13X14X15;
(peptide No. 16: SEQ ID NO: 12)
TNGQEVSASIDYNK;
(peptide No. 23: SEQ ID NO: 13)
AKLSNFASNDALPD;
(peptide No. 10: SEQ ID NO: 14)
PTTSSGASPDSSNP;
(peptide No. 21: SEQ ID NO: 15)
GLGRDLFVHSMGDK;
(peptide No. 15: SEQ ID NO: 16)
QIGKIKLSDVLSAS;
(peptide No. 34: SEQ ID NO: 17)
YGAIDKELHFSGGK;
(peptide No. 20: SEQ ID NO: 18)
NVDNILNMPSTTSG;
(peptide No. 22: SEQ ID NO: 19)
GNLKGVYYPKSSTT;
(peptide No. 14: SEQ ID NO: 20)
ITEKIQSGKLTITI;
(peptide No. 26: SEQ ID NO: 21)
FHDFLVSLKGKKFA;
(peptide No. 31: SEQ ID NO: 22)
TTGGEVRLFRSFYV;
and
(peptide No. 35: SEQ ID NO: 23)
IGARFGLDYQDINI,

or an immunologically equivalent mutant thereof, wherein

X1 is K or D, X2 is Q, E or T, X3 is Q or S, X4 is M or L, X5 is E or K, X6 is P or S, X7 is D or G, X8 is N or T, X9 is L or F, X10 is N or D, X11 is G, D or N, X12 is Q or M, X13 is M or L, X14 is G or N, and X15 is L or M.

(6) The peptide or the immunologically equivalent mutant thereof of (5), wherein the HsvA antigen peptide is a peptide having any one amino acid sequence selected from the group of the following:

(peptide No. 11: SEQ ID NO: 3)
EKKAVQQMENSNPD;
(peptide No. 11 (TKY): SEQ ID NO: 4)
EKKAVEQMENSNPD;
(peptide No. 11 (SH8): SEQ ID NO: 5)
EKDAVTSLKNSNSG;
(peptide No. 11 (SH10): SEQ ID NO: 6)
EKDAVTSLENSNSG;
(peptide No. 19: SEQ ID NO: 7)
NQGTLEFLSNDVSN;
(peptide No. 19 (TKY): SEQ ID NO: 8)
NQGTLEFLSNDVST;
(peptide No. 33: SEQ ID NO: 9)
LSNKLQGQLKSMGL;
(peptide No. 33 (TKY): SEQ ID NO: 10)
LSNKLQDQLKSMGL;
(peptide No. 33 (SH10): SEQ ID NO: 11)
FSDKLQNMLKSLNM;
(peptide No. 16: SEQ ID NO: 12)
TNGQEVSASIDYNK;
(peptide No. 23: SEQ ID NO: 13)
AKLSNFASNDALPD;
(peptide No. 10: SEQ ID NO: 14)
PTTSSGASPDSSNP;
(peptide No. 21: SEQ ID NO: 15)
GLGRDLFVHSMGDK;
(peptide No. 15: SEQ ID NO: 16)
QIGKIKLSDVLSAS;
(peptide No. 34: SEQ ID NO: 17)
YGAIDKELHFSGGK;
(peptide No. 20: SEQ ID NO: 18)
NVDNILNMPSTTSG;
(peptide No. 22: SEQ ID NO: 19)
GNLKGVYYPKSSTT;
(peptide No. 14: SEQ ID NO: 20)
ITEKIQSGKLTITI;
(peptide No. 26: SEQ ID NO: 21)
FHDFLVSLKGKKFA;
(peptide No. 31: SEQ ID NO: 22)
TTGGEVRLFRSFYV;
(peptide No. 35: SEQ ID NO: 23)
IGARFGLDYQDINI;
(peptide No. 8: SEQ ID NO: 86)
KQLPQPKRSELKPK;
(peptide No. 31N: SEQ ID NO: 87)
TNIKQYMQNNHRSQ;
(peptide No. 81: SEQ ID NO: 88)
TLTLEGTETFAQNS;
(peptide No. 63: SEQ ID NO: 89)
EAYAKNQGDIWSTI;
(peptide No. 73: SEQ ID NO: 90)
VIGSKSSITLNSAN;
and
(peptide No. 61: SEQ ID NO: 91)
ADIQSSQTTFANSV;

or an immunologically equivalent mutant thereof.
(7) An antibody that specifically binds to a peptide or an immunologically equivalent mutant thereof of any one of (1) to (6), or an immunoreactive fragment thereof.
(8) A nucleic acid molecule consisting of 5 to 40 nucleotides, the nucleic acid molecule being capable of binding under stringent conditions to hsvA gene.
(9) The nucleic acid molecule of (8), wherein the hsvA gene has the nucleotide sequence set forth in SEQ ID NO: 1, SEQ ID NO: 27 or SEQ ID NO: 29.
(10) The nucleic acid molecule of (9) having any one nucleotide sequence selected from the group of the following:

(SEQ ID NO: 31)
CTTTTAGCGTGGTATCCTTC;
(SEQ ID NO: 32)
TTACAAAGACCACCAACGGA;
(SEQ ID NO: 33)
TACCATAGAAAGCGGAAAC;
(SEQ ID NO: 34)
AGCTATAGCTTTGCACCTGA;
(SEQ ID NO: 35)
ACTAACAGCATAGAAGTTGGGGA;
(SEQ ID NO: 36)
AACAACATTACAGGCATGAG;
(SEQ ID NO: 37)
CAAATAGTAACAGGACAATT;
(SEQ ID NO: 38)
CAGGATTTAAGAGGCACTTA;
(SEQ ID NO: 39)
TCTTAACCAATCTGAGCAA;
(SEQ ID NO: 40)
GTCAAACACGTTATTGATGGCTT;
(SEQ ID NO: 41)
GGGGTTTAATAGCATAGGG;
(SEQ ID NO: 42)
TAGAGCTCTACACCAGTCTT;
(SEQ ID NO: 43)
ATAAAGCCCATGAATTCTTAGGCATGCGTGCTCTG;
(SEQ ID NO: 44)
TGCTATTGATAAAGAGTTACATTTCTCAGG;
(SEQ ID NO: 45)
ATTTCTCAGGGGGAAAGTC;
(SEQ ID NO: 46)
GGCTAATAATTTAACCACAATAAGCGCCTTTAA;
(SEQ ID NO: 47)
GATGGGCGCTTCTGGTTTA;
(SEQ ID NO: 48)
ATGAATTCTTAGGCATGCGTGCTCT;
(SEQ ID NO: 49)
TTTTGGAGGTGTAGGAGTAGAT;
(SEQ ID NO: 50)
AGCGCGCATTTAAAACAGGT;
(SEQ ID NO: 51)
AGAAACGAGATTACAGGAAG;
(SEQ ID NO: 52)
AGGAGCAAGTTTTGTAGCAG;
(SEQ ID NO: 53)
AAAATGCGACTGATTGGATG;
(SEQ ID NO: 54)
TTGAAATTTGGCCACGCT;
(SEQ ID NO: 55)
TCACCCATAGAATGGACGAA;
(SEQ ID NO: 56)
CTAGCGCATTAACCACAGACTG;
(SEQ ID NO: 57)
TAGAAGTTGTAGACACGGT;
(SEQ ID NO: 58)
GTGATATTGCCTTTCTGAAC;
(SEQ ID NO: 59)
GCAAGTTTTGTGCGGATT;
(SEQ ID NO: 60)
ATGTGATACACATCTGACC;
(SEQ ID NO: 61)
AGTGCCGTTACCATCGTGAA;
(SEQ ID NO: 62)
TATTCAAGGAAAGTCCCTGGAGAAACTCCAGAGAC;
(SEQ ID NO: 63)
TTAAAGGCGCTTATTGTGGTTAAATTATTAGCC;
(SEQ ID NO: 64)
ATTAGCCTTAAGGGTGCTATC;
(SEQ ID NO: 65)
CTGGTAATGCATCATTAGAAGCAAA;
(SEQ ID NO: 66)
TACGCGCAAAATAGGTTCTT;
(SEQ ID NO: 67)
TGGAGAAACTCCAGAGACTA;
(SEQ ID NO: 68)
TTGTTCGCTGTAGTGCCGTGG;
(SEQ ID NO: 69)
GAAGGATACCACGCTAAAAG;
(SEQ ID NO: 70)
AAGCTAGAGTTTTGGTTGAG;
(SEQ ID NO: 71)
TTGCTCAGATTGGTTAAGA;
(SEQ ID NO: 72)
ATCGAAATAAGCGAACCTCA;
(SEQ ID NO: 73)
TTGAAAGCTTAGCTAAACGG;
(SEQ ID NO: 74)
TGGTATTGCTGGTTAAGAGG,
(SEQ ID NO: 75)
CAAACAGATGAGCCGT;
(SEQ ID NO: 76)
ATGAAAAAGTTTAGTTCTCTCACATTGAAATTTGGCCACGCTC;
and
(SEQ ID NO: 77)
CTAAAAAGCATAGCGCATCCCGACATTGCCTGTAATATTAATATC.

(11) The nucleic acid molecule of any one of (8) to (10), wherein the nucleic acid molecule is a primer capable of amplifying the whole or a portion of the hsvA gene.
(12) The nucleic acid molecule of any one of (8) to (10), wherein the nucleic acid molecule is a probe for detecting the hsvA gene.
(13) A method for determining the presence of H. suis, comprising detecting the whole or a portion of hsvA gene in a sample, wherein the sample in which the whole or a portion of the hsvA gene has been detected is determined to have H. suis.
(14) The method for determining the presence of H. suis of (13), comprising:

amplifying the whole or a portion of the hsvA gene using DNA in the sample as a template and using a primer of (11);

detecting the amplified DNA; and

determining the sample in which the DNA amplification has been detected, to have H. suis.

(15) The method for determining the presence of H. suis of (13), comprising:

contacting DNA in the sample with a probe of (12);

detecting DNA bound with the probe of (12); and

determining the sample in which the DNA bound with the probe of (11) has been detected, to have H. suis.

(16) A method for determining the presence of H. suis, comprising: detecting HsvA protein or a fragment thereof in a sample; and determining the sample in which the HsvA protein or the fragment thereof has been detected, to have H. suis.
(17) The method for determining the presence of H. suis of (16), comprising:

contacting the sample with an antibody or an immunoreactive fragment thereof of (7);

detecting the HsvA protein or the fragment thereof bound with the antibody or the immunoreactive fragment thereof, in the sample; and

determining the sample in which the HsvA protein or the fragment thereof bound with the antibody or the immunoreactive fragment thereof has been detected, as a sample having H. suis.

(18) A method for determining infection of a subject with H. suis, comprising:

performing a method of any one of (12) to (17) using a sample derived from the subject as a sample; and

determining the subject from which the sample determined to have H. suis by the method is derived, to be infected with H. suis.

(19) A method for determining infection of a subject with H. suis, comprising:

detecting an antibody that binds to an HsvA antigen peptide, in a blood sample derived from the subject; and

determining the subject in which the antibody that binds to the peptide has been detected, to be infected with H. suis.

(20) The method for determining infection with H. suis of (19), comprising the steps of:

contacting the sample derived from the subject with a peptide of any one of (1) to (6);

detecting an antibody bound with the peptide, in the blood sample; and

determining the subject in which the antibody bound with the peptide has been detected, to be infected with H. suis.

(21) A composition for determination of H. suis infection, comprising any one member selected from a peptide of any one of (1) to (6), a nucleic acid molecule of any one of (8) to (10), and an antibody or an immunoreactive fragment thereof of (7).
(22) A pharmaceutical composition comprising any one member selected from a peptide of any one of (1) to (6), a nucleic acid molecule of any one of (8) to (10), and an antibody or an immunoreactive fragment thereof of (7).
(23) The pharmaceutical composition of (22) for H. suis eradication therapy.
(24) The pharmaceutical composition of (22) for treatment or prevention of gastritis, gastric ulcer, duodenal ulcer, stomach cancer, chronic gastritis, gastric MALT lymphoma, nodular gastritis, idiopathic thrombocytopenic purpura, functional dyspepsia, or diffuse large B-cell lymphoma.

(H. suis)

The term “H. suis” herein means a strain that is included in H. heilmannii (in a broad sense) and belongs to H. heilmannii type 1. H. suis is known to infect the stomachs of humans as well as dogs, cats, pigs, monkeys, and the like. An infected mammal herein from which the H. suis has been isolated is not particularly limited and can be, for example, a human, a monkey, or a pig. Preferably, H. suis is H. suis isolated from a human (e.g., an SNTW101 strain).

(hsvA Gene and HsvA Protein)

The terms “hsvA gene” and “HsvA protein” herein mean an H. suis-derived gene encoding or amino acids having an amino-terminal signal peptide region, a carboxy-terminal autotransporter (outer membrane transport mechanism of a protein) β-domain and a disordered region involved in the exertion of functions, in an outer membrane protein of a bacterium of the Helicobacter genus. One non-limiting example of the hsvA gene and the HsvA protein can include a gene having the nucleotide sequence set forth in SEQ ID NO: 1 (or the nucleotide sequence of FIG. 2; the same holds true herein), and a protein having the amino acid sequence set forth in SEQ ID NO: 2 (or the amino acid sequence of FIG. 2; the same holds true herein), respectively, both of which are derived from an H. suis SNTW101 strain. In the amino acid sequence set forth in SEQ ID NO: 2, amino acid positions 1 to 27 correspond to a signal sequence, amino acid positions 2273 to 2475 correspond to a disordered region, and amino acid positions 2686 to 2992 correspond to an autotransporter β-region. Accordingly, the HsvA protein herein includes a protein consisting of amino acid positions 28 to 2992 excluding the signal sequence in the amino acid sequence set forth in SEQ ID NO: 2. Other hsvA genes and HsvA proteins derived from other strains of H. suis are also included in the hsvA gene and the HsvA protein herein. As one example, the HsvA protein includes an H. suis-derived protein having an amino acid sequence having 80%, 85%, 90%, 95%, 98%, or 99% identity to the amino acid sequence set forth in SEQ ID NO: 2 (or an amino acid sequence consisting of amino acid positions 28 to 2992 in the amino acid sequence set forth in SEQ ID NO: 2). Likewise, the hsvA gene includes an H. suis-derived gene having a nucleotide sequence encoding an amino acid sequence having 80%, 85%, 90%, 95%, 98%, or 99% identity to the amino acid sequence set forth in SEQ ID NO: 2 (or an amino acid sequence consisting of amino acid positions 28 to 2992 in the amino acid sequence set forth in SEQ ID NO: 2). In this context, the identity of an amino acid sequence can be determined by a method usually used by those skilled in the art, for example, using a known program such as BLAST or FASTA. Alternatively, the hsvA gene includes an H. suis-derived gene comprising DNA hybridizing under stringent conditions to DNA having a nucleotide sequence complementary to the nucleotide sequence set forth in SEQ ID NO: 1. The HsvA protein includes a protein encoded by this gene. The HsvA protein further includes an H. suis-derived protein having an amino acid sequence derived from the amino acid sequence set forth in SEQ ID NO: 2 (or an amino acid sequence consisting of amino acid positions 28 to 2992 in the amino acid sequence set forth in SEQ ID NO: 2) by the substitution with other amino acids, deletion, addition, or insertion of 1 to 50 (which may be, for example, 1 to 40, 1 to 30, 1 to 25, 1 to 20, 1 to 15, 1 to 10, 1 to 8, 1 to 6, 1 to 5, 1 to 4, 1 to 3, or 1 to 2; the same holds true herein) amino acids. Likewise, the hsvA gene includes an H. suis-derived gene having a nucleotide sequence encoding an amino acid sequence derived from the amino acid sequence set forth in SEQ ID NO: 2 (or an amino acid sequence consisting of amino acid positions 28 to 2992 in the amino acid sequence set forth in SEQ ID NO: 2) by the substitution with other amino acids, deletion, addition, or insertion of 1 to 50 amino acids.

The hsvA gene and the HsvA protein include, for example, a gene having the nucleotide sequence set forth in SEQ ID NO: 27 or 29, and the amino acid sequence set forth in SEQ ID NO: 28 or 30, which are derived from an H. suis HS1 or HS5 strain, respectively.

Preferably, the HsvA protein has any one of the amino acid sequences set forth in SEQ ID NOs: 78 to 83; an amino acid sequence having 80%, 85%, 90%, 95%, 98%, or 99% identity to any one of the amino acid sequences set forth in SEQ ID NOs: 78 to 83; or an amino acid sequence derived from any one of the amino acid sequences set forth in SEQ ID NOs: 78 to 83 by the substitution with other amino acids, deletion, addition, or insertion of 1 to 50 amino acids.

(HsvA Antigen Peptide)

The term “HsvA antigen peptide” herein means a peptide having an amino acid sequence contained in the HsvA protein mentioned above, the peptide being capable of functioning as an antigen in vivo. Preferably, the peptide has an amino acid sequence conserved among a plurality of H. suis strains. Preferably, the HsvA antigen peptide consists of an H. suis-specific amino acid sequence absent in H. pylon or other H. heilmannii. Since HsvA is a membrane protein, preferably, the HsvA antigen peptide comprises an amino acid sequence constituting the extracellular domain of the HsvA protein. The HsvA antigen peptide consists of, for example, an amino acid sequence contained in a sequence from positions 1467 to 2992, positions 1467 to 2685, positions 1535 to 2135, or positions 2008 to 2135 in the amino acid sequence set forth in SEQ ID NO: 2 of the HsvA protein derived from an H. suis SNTW101 strain. As a result of analyzing HsvA-specific amino acid sequences that satisfy such conditions, the present inventors have successfully found the partial amino acid sequences set forth in SEQ ID NOs: 78 to 83. The amino acid sequences set forth in SEQ ID NOs: 78 to 83 are of HsvA proteins derived from different strains of H. suis infecting different species. These amino acid sequences are relatively conserved and have 76.7% identity. Accordingly, the HsvA protein antigen peptide of the present invention can have an amino acid sequence having 70%, 75%, 80%, 85%, 90%, 95%, 98%, or 99% identity to any one of the amino acid sequences set forth in SEQ ID NOs: 78 to 83; or an amino acid sequence derived from any one of the amino acid sequences set forth in SEQ ID NOs: 78 to 83 by the substitution with other amino acids, deletion, addition, or insertion of 1 to 50 amino acids, or can have an amino acid sequence contained in this amino acid sequence. The phrase “having a (particular) amino acid sequence” herein is meant to also include “consisting of the amino acid sequence”.

The number of amino acids constituting the HsvA antigen peptide can be appropriately selected according to a purpose and may be, for example, 5 to 50 amino acids, 5 to 45 amino acids, 5 to 40 amino acids, 5 to 35 amino acids, 5 to 30 amino acids, 5 to 25 amino acids, 5 to 20 amino acids, 5 to 15 amino acids, 10 to 50 amino acids, 10 to 45 amino acids, 10 to 40 amino acids, 10 to 35 amino acids, 10 to 30 amino acids, 10 to 25 amino acids, or 10 to 20 amino acids. One example of the HsvA antigen peptide can include a peptide having the whole or a portion of an amino acid sequence contained in EKX1AVX2X3X4X5NSNX6X7 (peptide No. 11 (mix): SEQ ID NO: 24) (wherein X1 is K or D, X2 is Q, E or T, X3 is Q or S, X4 is M or L, X5 is E or K, X6 is P or S, and X7 is D or G), EKKAVQQMENSNPD (peptide No. 11 (SNTW101; HS1; HS5): SEQ ID NO: 3), EKKAVEQMENSNPD (peptide No. 11 (TKY): SEQ ID NO: 4), EKDAVTSLKNSNSG (peptide No. 11 (SH8): SEQ ID NO: 5), EKDAVTSLENSNSG (peptide No. 11 (SH10): SEQ ID NO: 6), NQGTLEFLSNDVSX8 (peptide No. 19: SEQ ID NO: 25) (wherein X8 is N or T), NQGTLEFLSNDVSN (peptide No. 19: SEQ ID NO: 7), NQGTLEFLSNDVST (peptide No. 19 (TKY): SEQ ID NO: 8), X9SX10KLQX11X12LKSX13X14X15 (peptide No. 33: SEQ ID NO: 26) (wherein X9 is L or F, X10 is N or D, X11 is G, D or N, X12 is Q or M, X13 is M or L, X14 is G or N, and X15 is L or M), LSNKLQGQLKSMGL (peptide No. 33: SEQ ID NO: 9), LSNKLQDQLKSMGL (peptide No. 33 (TKY): SEQ ID NO: 10), FSDKLQNMLKSLNM (peptide No. 33 (SH10): SEQ ID NO: 11), TNGQEVSASIDYNK (peptide No. 16: SEQ ID NO: 12), AKLSNFASNDALPD (peptide No. 23: SEQ ID NO: 13), PTTSSGASPDSSNP (peptide No. 10: SEQ ID NO: 14), GLGRDLFVHSMGDK (peptide No. 21: SEQ ID NO: 15), QIGKIKLSDVLSAS (peptide No. 15: SEQ ID NO: 16), YGAIDKELHFSGGK (peptide No. 34: SEQ ID NO: 17), NVDNILNMPSTTSG (peptide No. 20: SEQ ID NO: 18), GNLKGVYYPKSSTT (peptide No. 22: SEQ ID NO: 19), ITEKIQSGKLTITI (peptide No. 14: SEQ ID NO: 20), FHDFLVSLKGKKFA (peptide No. 26: SEQ ID NO: 21), TTGGEVRLFRSFYV (peptide No. 31: SEQ ID NO: 22), IGARFGLDYQDINI (peptide No. 35: SEQ ID NO: 23), KQLPQPKRSELKPK (peptide No. 8: SEQ ID NO: 86), TNIKQYMQNNHRSQ (peptide No. 31N: SEQ ID NO: 87), TLTLEGTETFAQNS (peptide No. 81: SEQ ID NO: 88), EAYAKNQGDIWSTI (peptide No. 63: SEQ ID NO: 89), VIGSKSSITLNSAN (peptide No. 73: SEQ ID NO: 90), and ADIQSSQTTFANSV (peptide No. 61: SEQ ID NO: 91). The HsvA antigen peptide may have one to several amino acids substituted, deleted, added or inserted in the peptide sequence as long as the peptide exerts functions as the HsvA antigen of interest. In the substitution of an amino acid, preferably, the amino acid is conservatively substituted with a similar amino acid residue. Examples thereof can include substitution between amino acids such as G and P, G and A or V, L and I, E and Q, D and N, C and T, T and S or A, and K and R.

The amino acid herein is described in a single-letter code. Specifically, C represents cysteine, A represents alanine, Y represents tyrosine, H represents histidine, R represents arginine, G represents glycine, E represents glutamic acid, L represents leucine, V represents valine, W represents tryptophan, T represents threonine, Y represents tyrosine, I represents isoleucine, F represents phenylalanine, D represents aspartic acid, N represents asparagine, Q represents glutamine, M represents methionine, K represents lysine, and P represents proline. Xn (n is a natural number) herein represents one type of amino acid selected from the group consisting of a plurality of amino acids defined respectively. For example, if “X1” is K or D in EKX1AVX2X3X4X5NSNX6X7 (SEQ ID NO: 24), that means the amino acid represented by X1 is either lysine or aspartic acid.

In one aspect, the present invention relates to an immunologically equivalent mutant of the HsvA antigen peptide. The term “Immunologically equivalent mutant of the peptide” herein means a peptide capable of binding to an antibody that binds to a peptide having a sequence derived from natural HsvA protein, but has an amino acid sequence different from that of the peptide having a sequence derived from natural HsvA protein. The mutant may be, for example, a peptide having one to several amino acids substituted with natural or artificial amino acids, deleted, added, or inserted in the peptide sequence. Alternatively, the mutant may be a peptide having some modified amino acids constituting the peptide having a sequence derived from natural HsvA protein. Alternatively, both the mutations, i.e., the amino acid mutation and the modification, may be introduced therein. The binding to an antibody that binds to a peptide having a sequence derived from natural HsvA protein means binding to an anti-HsvA antibody in other words. For example, a peptide capable of binding to an anti-HsvA antibody present in the blood of a patient infected with H. suis is included in the immunologically equivalent mutant of the peptide of the present invention. Whether or not a test peptide mutant is capable of binding to the antibody that binds to a peptide having a sequence derived from natural HsvA protein, or the anti-HsvA antibody present in the blood of a patient infected with H. suis can be determined by reacting the antibody with a solid phase bound with the peptide mutant, washing the solid phase, and then further reacting a labeled anti-IgG antibody therewith. When labeled IgG bound with the solid phase is detected after washing of labeled IgG, this peptide is determined as the immunologically equivalent mutant of the peptide of the present invention. The term “HsvA antigen peptide” herein includes the immunologically equivalent mutant of the HsvA antigen peptide, unless otherwise specified.

(Anti-HsvA Antibody or Immunoreactive Fragment Thereof)

In another aspect, the present invention relates to an antibody specifically recognizing HsvA protein (referred to as “anti-HsvA antibody” herein) or an immunoreactive fragment thereof. The anti-HsvA antibody of the present invention preferably binds to any of the HsvA protein antigen peptides defined above. The anti-HsvA antibody binds to, for example, a peptide having any one of the amino acid sequences set forth in SEQ ID NOs: 78 to 83; an amino acid sequence having 80%, 85%, 90%, 95%, 98%, or 99% identity to any one of the amino acid sequences set forth in SEQ ID NOs: 78 to 83; or an amino acid sequence derived from any one of the amino acid sequences set forth in SEQ ID NOs: 78 to 83 by the substitution with other amino acids, deletion, addition, or insertion of 1 to 50 amino acids. “Immunoreactive fragment of the antibody” means a portion of the antibody (partial fragment) or a peptide comprising a portion of the antibody, which retains the binding effect of the antibody to the antigen. Examples of such a fragment of the antibody can include F(ab′)2, Fab′, Fab, single-chain Fv (hereinafter, referred to as “scFv”), disulfide-stabilized Fv (hereinafter, referred to as “dsFv”) and their polymers, dimerized V region (hereinafter, referred to as “Diabody”), and a peptide comprising CDRs. Preferably, the antibody of the present invention or the immunoreactive fragment thereof specifically recognizes the HsvA antigen peptide. The phrase ““specifically” recognizes (binds)” herein for the antibody or the immunoreactive fragment thereof means that the antibody or the immunoreactive fragment thereof binds to the HsvA antigen peptide with substantially higher affinity than that for other amino acid sequences or conformations. The association constant (Ka) of the binding of the antibody of the present invention to the HsvA antigen can include, for example, at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, and at least 10¹³ M⁻¹.

The antibody of the present invention may be polyclonal or monoclonal as long as the antibody can specifically recognize the HsvA antigen peptide. Preferably, the antibody of the present invention is monoclonal antibody. In this context, “monoclonal antibody” means an antibody derived from a single clone. The antibody of the present invention includes a complete antibody as well as a bispecific antibody and a single-chain antibody as long as the bispecific antibody and the single-chain antibody retain binding activity against the antigen. The complete antibody encompasses a nonhuman animal antibody, an antibody having the amino acid sequence of a nonhuman animal antibody and the amino acid sequence of a human-derived antibody, and a human antibody. Examples of the nonhuman animal antibody can include mouse, rat, hamster, and rabbit antibodies. The nonhuman animal antibody is preferably an antibody of an animal from which a hybridoma can be prepared, more preferably a mouse antibody. Examples of the antibody having the amino acid sequence of a nonhuman animal antibody and the amino acid sequence of a human-derived antibody can include a human-type chimeric antibody composed of a human antibody grafted with antigen binding domain Fv of an animal-derived monoclonal antibody, and a humanized antibody in which the CDR sequences of Fv domains directly involved in the antigen binding of an animal-derived monoclonal antibody are incorporated in the framework regions of a human antibody. The human antibody is a human antibody which is an expression product of a completely human-derived antibody gene. The immunoglobulin class of the antibody of the present invention is not particularly limited and may be any of immunoglobulin classes IgG, IgM, IgA, IgE, IgD, and IgY. Preferably, the immunoglobulin class of the antibody of the present invention is IgG. The antibody of the present invention also encompasses an antibody of any isotype.

The HsvA antigen peptide, or the anti-HsvA antibody or the immunoreactive fragment thereof may be labeled, if necessary. A detectable label such as a radiolabel, an enzyme, a fluorescent label, a bioluminescent label, a chemiluminescent label, metal can be used as the label. Examples of such a label can include, but are not limited to, detectable labels including: radiolabels such as 35S (sulfur 35), 32P (phosphorus 32), 3H (tritium), 125I (iodine 125), 131I (iodine 131), and 14C (carbon 14); enzymes such as β galactosidase, peroxidase, alkaline phosphatase, glucose oxidase, lactate oxidase, alcohol oxidase, monoamine oxidase, and horse radish peroxidase; coenzymes or prosthetic groups such as FAD, FMN, ATP, biotin, and hem; fluorescent labels such as fluorescein derivatives (fluorescein isothiocyanate (FITC), fluorescein thiofulbamil, etc.), rhodamine derivatives (tetramethylrhodamine, trimethylrhodamine (RITC), Texas Red, rhodamine 110, etc.), Cy dyes (Cy3, Cy5, Cy5.5, and Cy7), Cy-chrome, Spectrum Green, Spectrum Orange, propidium iodide, allophycocyanin (APC), R-phycoerythrin (R-PE), Alexa-Flour® fluorescent dyes such as Alexa-Flour® 488 and Alexa-Flour® 568 (Molecular Probes, Inc., USA), and Alexa-Flour® 568; bioluminescent labels such as luciferase; chemiluminescent labels such as luminol, luminol derivatives such as isoluminol and N-(4-aminobutyl)-N-ethyl isoluminol ester, acridinium derivatives such as N-methyl acridinium ester and N-methyl acridinium acylsulfonamide ester, lucigenin, adamantyl dioxetane, indoxyl derivatives, and ruthenium complexes; and metals such as metal colloids.

(Nucleic Acid Molecule, Primer, and Probe Having hsvA Gene-Specific Nucleotide Sequence)

In an alternative aspect, the present invention relates to a nucleic acid molecule capable of specifically binding to hsvA gene. Preferably, the nucleic acid molecule capable of specifically binding to hsvA gene relates to a nucleic acid molecule having an hsvA gene-specific nucleotide sequence or a sequence complementary to the nucleotide sequence, or a nucleic acid molecule capable of binding under stringent conditions to an hsvA gene-specific nucleotide sequence or a sequence complementary to the nucleotide sequence (hereinafter, referred to as “nucleic acid molecule having an hsvA gene-specific nucleotide sequence, etc.”). The hsvA-specific nucleic acid molecule may be a primer or a probe according to an application. In this context, the primer is a nucleic acid molecule that is used for the purpose of amplifying DNA by PCR or the like. The primer is a nucleic acid molecule that hybridizes to DNA having a sequence complementary to the primer so that the DNA can be elongated and thereby amplified. The DNA is detectable by detecting the amplified DNA. The probe is a nucleic acid molecule that is used for the purpose of detecting the presence of target DNA through binding to the target DNA. The nucleic acid molecule capable of binding to hsvA gene of the present invention may have a nucleotide sequence that is not derived from hsvA in the nucleotide sequence of the hsvA gene as long as the sequence is not identical or similar to that of H. pylori and H. heilmannii (except for H. suis). Whether or not a selected nucleotide sequence is specific for the hsvA gene can be determined, for example, by searching an already published database for this sequence, examining whether or not H. pylori and H. heilmannii (except for H. suis) have an identical or similar sequence, and determining the sequence to be not specific when the identical or similar sequence is present and to be specific when the identical or similar sequence is absent. In this context, the phrase “not similar” may mean that the identity is 10% or lower, 20% or lower, 30% or lower, 40% or lower, or 50% or lower. The nucleic acid molecule capable of specifically binding to hsvA gene can be selected as an arbitrary sequence, for example, from SEQ ID NO: 1, according to the method described above. The nucleic acid molecule capable of binding to hsvA gene of the present invention can be of, for example, 5 to 50 nucleotides, 5 to 45 nucleotides, 5 to 40 nucleotides, 5 to 35 nucleotides, 5 to 30 nucleotides, 5 to 25 nucleotides, 5 to 20 nucleotides, 5 to 15 nucleotides, 5 to 10 nucleotides, 10 to 50 nucleotides, 10 to 45 nucleotides, 10 to 40 nucleotides, 10 to 35 nucleotides, 10 to 30 nucleotides, 10 to 25 nucleotides, 10 to 20 nucleotides, 10 to 15 nucleotides, 15 to 50 nucleotides, 15 to 45 nucleotides, 15 to 40 nucleotides, 15 to 35 nucleotides, 15 to 30 nucleotides, 15 to 25 nucleotides, 15 to 20 nucleotides, or 17 to 25 nucleotides. Preferably, examples of the hsvA gene-specific nucleotide sequence can include

(SEQ ID NO: 31)
CTTTTAGCGTGGTATCCTTC,
(SEQ ID NO: 32)
TTACAAAGACCACCAACGGA,
(SEQ ID NO: 33)
TACCATAGAAAGCGGAAAC,
(SEQ ID NO: 34)
AGCTATAGCTTTGCACCTGA,
(SEQ ID NO: 35)
ACTAACAGCATAGAAGTTGGGGA,
(SEQ ID NO: 36)
AACAACATTACAGGCATGAG,
(SEQ ID NO: 37)
CAAATAGTAACAGGACAATT,
(SEQ ID NO: 38)
CAGGATTTAAGAGGCACTTA,
(SEQ ID NO: 39)
TCTTAACCAATCTGAGCAA,
(SEQ ID NO: 40)
GTCAAACACGTTATTGATGGCTT,
(SEQ ID NO: 41)
GGGGTTTAATAGCATAGGG,
(SEQ ID NO: 42)
TAGAGCTCTACACCAGTCTT,
(SEQ ID NO: 43)
ATAAAGCCCATGAATTCTTAGGCATGCGTGCTCTG,
(SEQ ID NO: 44)
TGCTATTGATAAAGAGTTACATTTCTCAGG,
(SEQ ID NO: 45)
ATTTCTCAGGGGGAAAGTC,
(SEQ ID NO: 46)
GGCTAATAATTTAACCACAATAAGCGCCTTTAA,
(SEQ ID NO: 47)
GATGGGCGCTTCTGGTTTA,
(SEQ ID NO: 48)
ATGAATTCTTAGGCATGCGTGCTCT,
(SEQ ID NO: 49)
TTTTGGAGGTGTAGGAGTAGAT,
(SEQ ID NO: 50)
AGCGCGCATTTAAAACAGGT,
(SEQ ID NO: 51)
AGAAACGAGATTACAGGAAG,
(SEQ ID NO: 52)
AGGAGCAAGTTTTGTAGCAG,
(SEQ ID NO: 53)
AAAATGCGACTGATTGGATG,
(SEQ ID NO: 54)
TTGAAATTTGGCCACGCT,
(SEQ ID NO: 55)
TCACCCATAGAATGGACGAA,
(SEQ ID NO: 56)
CTAGCGCATTAACCACAGACTG,
(SEQ ID NO: 57)
TAGAAGTTGTAGACACGGT,
(SEQ ID NO: 58)
GTGATATTGCCTTTCTGAAC,
(SEQ ID NO: 59)
GCAAGTTTTGTGCGGATT,
(SEQ ID NO: 60)
ATGTGATACACATCTGACC,
(SEQ ID NO: 61)
AGTGCCGTTACCATCGTGAA,
(SEQ ID NO: 62)
TATTCAAGGAAAGTCCCTGGAGAAACTCCAGAGAC,
(SEQ ID NO: 63)
TTAAAGGCGCTTATTGTGGTTAAATTATTAGCC,
(SEQ ID NO: 64)
ATTAGCCTTAAGGGTGCTATC,
(SEQ ID NO: 65)
CTGGTAATGCATCATTAGAAGCAAA,
(SEQ ID NO: 66)
TACGCGCAAAATAGGTTCTT,
(SEQ ID NO: 67)
TGGAGAAACTCCAGAGACTA,
(SEQ ID NO: 68)
TTGTTCGCTGTAGTGCCGTGG,
(SEQ ID NO: 69)
GAAGGATACCACGCTAAAAG,
(SEQ ID NO: 70)
AAGCTAGAGTTTTGGTTGAG,
(SEQ ID NO: 71)
TTGCTCAGATTGGTTAAGA,
(SEQ ID NO: 72)
ATCGAAATAAGCGAACCTCA,
(SEQ ID NO: 73)
TTGAAAGCTTAGCTAAACGG,
(SEQ ID NO: 74)
TGGTATTGCTGGTTAAGAGG,
(SEQ ID NO: 75)
CAAACAGATGAGCCGT,
(SEQ ID NO: 76)
ATGAAAAAGTTTAGTTCTCTCACATTGAAATTTGGCCACGCTC,
and
(SEQ ID NO: 77)
CTAAAAAGCATAGCGCATCCCGACATTGCCTGTAATATTAATATC.

In use of the nucleic acid molecule having an hsvA gene-specific nucleotide sequence as a primer, primers selected from the following primers can be used in combination as a forward primer and a reverse primer.

| (Forward primer) | |
|---|---|
| (SEQ ID NO: 51) | |
| HSVANOFW-2: | AGAAACGAGATTACAGGAAG |
| (SEQ ID NO: 52) | |
| HSVANOFW-3: | AGGAGCAAGTTTTGTAGCAG |
| (SEQ ID NO: 53) | |
| HSVANOFW-5: | AAAATGCGACTGATTGGATG |
| (Reverse primer) | |
| (SEQ ID NO: 72) | |
| HSVANORV-1: | ATCGAAATAAGCGAACCTCA |
| (SEQ ID NO: 73) | |
| HSVANORV-3: | TTGAAAGCTTAGCTAAACGG |
| (SEQ ID NO: 74) | |
| HSVANORV-4: | TGGTATTGCTGGTTAAGAGG. |

In use of the nucleic acid molecule having an hsvA gene-specific nucleotide sequence as a primer, the combination of the forward primer and the reverse primer is preferably a combination HSVANOFW-2/HSVANORV-4 (101 bp), HSVANOFW-3/HSVANORV-4 (291 bp), HSVANOFW-5/HSVANORV-4 (556 bp), HSVANOFW-2/HSVANORV-1 (508 bp), or HSVANOFW-5/HSVANORV-3 (108 bp) (the length of a DNA fragment obtained by PCR amplification using the primers is indicated within the parentheses).

The phrase “hybridizing under stringent conditions” herein means hybridizing under hybridization conditions usually used by those skilled in the art. Whether or not to hybridize can be determined by a method described in, for example, Molecular Cloning, a Laboratory Manual, Fourth Edition, Cold Spring Harbor Laboratory Press (2012), or Current Protocols in Molecular Biology, Wiley Online Library. The hybridization conditions may be, for example, conditions involving hybridization at 42° C. in 6×SSC (0.9 M NaCl, 0.09 M trisodium citrate) or 6×SSPE (3 M NaCl, 0.2 M NaH₂PO₄, 20 mM EDTA.2Na, pH 7.4) followed by washing with 0.5×SSC at 42° C.

A “specific” sequence herein means that the sequence is characteristic of the protein, the peptide, or the nucleic acid molecule, and means that the sequence is substantially absent in other proteins, peptides, or nucleic acid molecules. In this context, the phrase “substantially absent” includes not only the case where a completely identical sequence is absent, as well as the case where similar sequence to that of the objective molecule (hsvA gene or HsvA protein) is present in the other molecules (e.g., an H. pylon-derived component) but the presence of the similar sequence still allows discrimination in biological binding reaction.

The phrase “specifically recognizing” or “specifically binding” means binding to the substance of interest but not substantially binding to other substances. Whether or not a candidate substance binds to the substance of interest and/or substantially does not bind to other substances can be confirmed by adopting a method well known to those skilled in the art such as Southern hybridization, PCR, Western blotting, or ELISA according to the types of the candidate substance (a nucleic acid, an antibody, etc.) and the substance of interest (a nucleic acid, a protein, etc.). In Western blotting, a protein can be confirmed, for example, by dissolving the protein in a solution of 2% SDS, 10% glycerol, 50 mM Tris-HCl (pH 6.8), and 100 mM dithiothreitol, boiling the solution, and conducting Western blot analysis using an anti-His antibody and a test antibody. In Southern hybridization, PCR, or ELISA, the substance can be confirmed using a method mentioned later. In this context, the phrase “not substantially binding” includes sufficiently discriminably low binding of a test substance to an allegedly non-bindable (or non-recognizable) nucleic acid, protein or peptide when compared with binding to the substance of interest. For example, the rate of cross-reaction with other substances compared with the substance of interest may be 1% or less, 0.5% or less, 0.3% or less, 0.1% or less, 0.05% or less, or 0.03% or less. The cross-reactivity can be obtained by calculation according to {(Actually measured value (concentration) for binding to other substances to be compared/Added sample concentration of the other substances to be compared)×100%}. Particularly, the phrase “the antibody or the immunoreactive fragment thereof “specifically” recognizes (binds)” herein means that the antibody or the immunoreactive fragment thereof binds to the antigen with substantially high affinity compared with affinity for other amino acid sequences, or the antigen binds to the antibody or the immunoreactive fragment thereof with substantially high affinity compared with affinity for other antibodies or immunoreactive fragments thereof. The term “substantially high affinity” herein means high affinity detectable by discriminating the particular amino acid sequence from other amino acid sequences using a desired measurement apparatus or method. The substantially high affinity may be, for example, 3 or more times, 4 or more times, 5 or more times, 6 or more times, 7 or more times, 8 or more times, 9 or more times, or 10 or more times in terms of intensity (e.g., fluorescence intensity) detected by ELISA or EIA.

(Kit and Composition)

In an alternative aspect, the present invention relates to a kit for determination of H. suis infection, a composition for determination of H. suis infection, and a pharmaceutical composition, comprising the HsvA antigen peptide or the nucleic acid molecule capable of specifically binding to hsvA gene. The kit and the composition of the present invention may further comprise a carrier selected from the group consisting of a solid phase, a hapten, and an insoluble carrier.

The kit and the composition of the present invention containing the nucleic acid molecule capable of specifically binding to hsvA gene can be based on a known method using a nucleic acid. Examples of such a method can include: a method using hybridization; a method using FOR; and a method known as Invader® assay (see, for example, Kwiatkowski, R. W. et al., Mol. Diagn. (1999) 4: 353-364).

The kit and the composition of the present invention may comprise, for example, a solid phase or a hapten on which a labeled nucleic acid molecule capable of specifically binding to hsvA gene is immobilized, and optionally, a solid phase on which a substance specifically binding to the hapten is immobilized. The kit and the composition of the present invention may comprise the nucleic acid molecule capable of specifically binding to hsvA gene as a primer (or a primer pair) or a probe. Alternatively, the kit and the composition of the present invention can have an allele-specific probe having an hsvA gene-specific nucleotide sequence, etc. and a sequence complementary to a portion of a quenching probe (flap), an Invader probe having the hsvA gene-specific nucleotide sequence, etc., triplex-specific DNase, and a fluorescently labeled universal probe having the quenching probe. The flap preferably differs among allele-specific probes. The fluorescent label can be appropriately selected for a probe well known to those skilled in the art and preferably differs among fluorescently labeled universal probes. For example, FAM and VIC can be used as fluorescent labels.

The kit and the composition containing the HsvA antigen peptide, or the antibody recognizing the peptide or the immunoreactive fragment thereof can be based on a known method using an antibody molecule. Examples of such a method can include: labeled immunoassay; immunoblotting; immunochromatography; chromatography; turbidimetric immunoassay (TIA); nanofluidic proteomic immunoassay (NIA); colorimetric method; latex immunoassay (LIA); counting immunoassay (CIA); chemiluminescent (enzyme) immunoassay (CLIA and CLEIA); precipitation reaction method; surface plasmon resonance (SPR) method; resonant mirror detector (RMD) method; and comparison interferometry. Whether or not the kit of the present invention is capable of carrying out the desired measurement can be confirmed by carrying out each measurement method by a method well known to those skilled in the art using the sample concerned or a sample of the concentration concerned, and thereby determining whether or not to be detectable.

The kit and the composition of the present invention can comprise, for example, (i) a solid phase or a hapten on which the HsvA antigen peptide, or the antibody recognizing the peptide or the immunoreactive fragment thereof is immobilized, and (ii) a labeled secondary antibody. The secondary antibody can be an anti-IgG antibody (in the case of immobilizing HsvA antigen peptide) or an anti-HsvA antibody (

in the case of immobilizing anti-HsvA antibody or immunoreactive fragment thereof)
according to the immobilized substance and an object to be detected. The kit and the composition of the present invention comprising a hapten may further comprise a solid phase on which a substance specifically binding to the hapten is immobilized.

In the kit and the composition of the present invention, the solid phase is not particularly limited as long as the solid phase can be used in immunochemical measurement. Examples thereof can include plates, tubes, chips (e.g., protein chips and Lab-on-a-Chip), beads, membranes, absorbers and/or particles containing nitrocellulose, Sepharose, nylon, vinylon, polyester, acryl, polyolefin, polyurethane, rayon, polynosic, cupra, lyocell, acetate, polyvinylidene difluoride, silicone rubber, latex, polystyrene, polypropylene, polyethylene, polyvinyl chloride, polyvinylidene chloride, polystyrene, polyvinyl acetate, fluorinated resin, ABS resin, AS resin, acrylic resin, polymer alloy, glass fiber, carbon fiber, glass, gelatin, a polyamino acid and/or a magnetically sensitive material. A plate and magnetic beads are preferred. The term “insoluble carrier” herein means a suspendable insoluble solid phase such as beads. Examples thereof can include latex beads and magnetic beads.

The kit and the composition of the present invention may comprise, if necessary, a coloring reagent, a reagent for reaction termination, a standard antigen reagent, a reagent for sample pretreatment, a blocking reagent, or the like. The kit and the composition of the present invention comprising a labeled antibody may further comprise a substrate reactive with the label. The kit of the present invention may appropriately comprise an attached document, an instruction, and a container for housing of the kit or the composition.

### Advantageous Effects of Invention

An HsvA antigen peptide and a nucleic acid molecule capable of specifically binding to hsvA gene can be used for measuring the presence or absence or antibody titer of an anti-H. suis antibody in blood derived from a subject to be diagnosed with H. suis infection. Use of the HsvA antigen peptide as a peptide vaccine is possible for prevention or treatment of H. suis infection. The HsvA antigen peptide can further be used as an antigen for obtaining an anti-HsvA antibody. An anti-HsvA antibody or an immunoreactive fragment thereof can be used in the detection of an HsvA antigen peptide. The anti-HsvA antibody or the immunoreactive fragment thereof can further be used as a therapeutic drug for treatment of H. suis infection.

### BRIEF DESCRIPTION OF DRAWINGS

FIG. 1 is a schematic view of an hsvA gene-specific nucleic acid molecule.

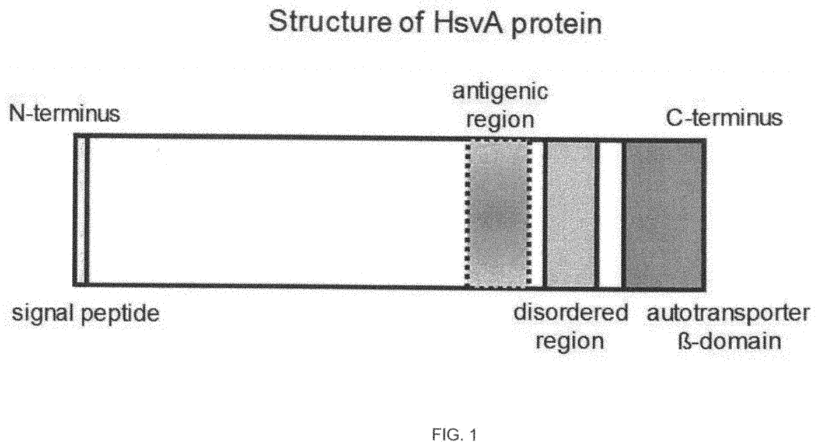

FIG. 2A shows the sequences of hsvA gene and HsvA protein derived from an SNTW101 strain of H. suis. FIGS. 2A to 2N show the consecutive sequences of one gene and a protein encoded thereby.

FIG. 2B shows the sequences of the hsvA gene and the HsvA protein derived from an SNTW101 strain of H. suis.

FIG. 2C shows the sequences of the hsvA gene and the HsvA protein derived from an SNTW101 strain of H. suis.

FIG. 2D shows the sequences of the hsvA gene and the HsvA protein derived from an SNTW101 strain of H. suis.

FIG. 2E shows the sequences of the hsvA gene and the HsvA protein derived from an SNTW101 strain of H. suis.

FIG. 2F shows the sequences of the hsvA gene and the HsvA protein derived from an SNTW101 strain of H. suis.

FIG. 2G shows the sequences of the hsvA gene and the HsvA protein derived from an SNTW101 strain of H. suis.

FIG. 2H shows the sequences of the hsvA gene and the HsvA protein derived from an SNTW101 strain of H. suis.

FIG. 2I shows the sequences of the hsvA gene and the HsvA protein derived from an SNTW101 strain of H. suis.

FIG. 2J shows the sequences of the hsvA gene and the HsvA protein derived from an SNTW101 strain of H. suis.

FIG. 2K shows the sequences of the hsvA gene and the HsvA protein derived from an SNTW101 strain of H. suis.

FIG. 2L shows the sequences of the hsvA gene and the HsvA protein derived from an SNTW101 strain of H. suis.

FIG. 2M shows the sequences of the hsvA gene and the HsvA protein derived from an SNTW101 strain of H. suis.

FIG. 2N shows the sequences of the hsvA gene and the HsvA protein derived from an SNTW101 strain of H. suis.

FIG. 3 is a diagram comparing the amino acid sequences of regions including peptide Nos. 11, 33, and 19 of SNTW101 (SEQ ID NO: 78), HS1 (SEQ ID NO: 79), HS5 (SEQ ID NO: 80), TKY (SEQ ID NO: 81), SH8 (SEQ ID NO: 82) and SH10 (SEQ ID NO: 83) strains of H. suis.

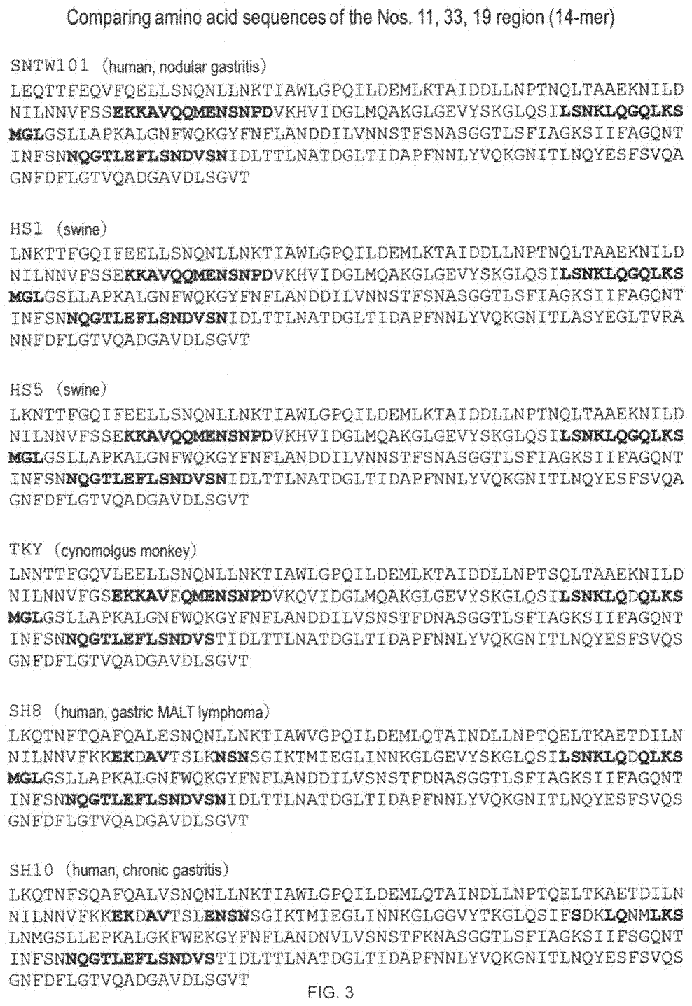

FIG. 4 is a diagram comparing the amino acid sequences of regions corresponding to peptide Nos. 11, 33, and 19 of SNTW101, HS1, HS5, TKY, SH8 and SH10 strains of H. suis (SEQ ID NOs: 3 to 11).

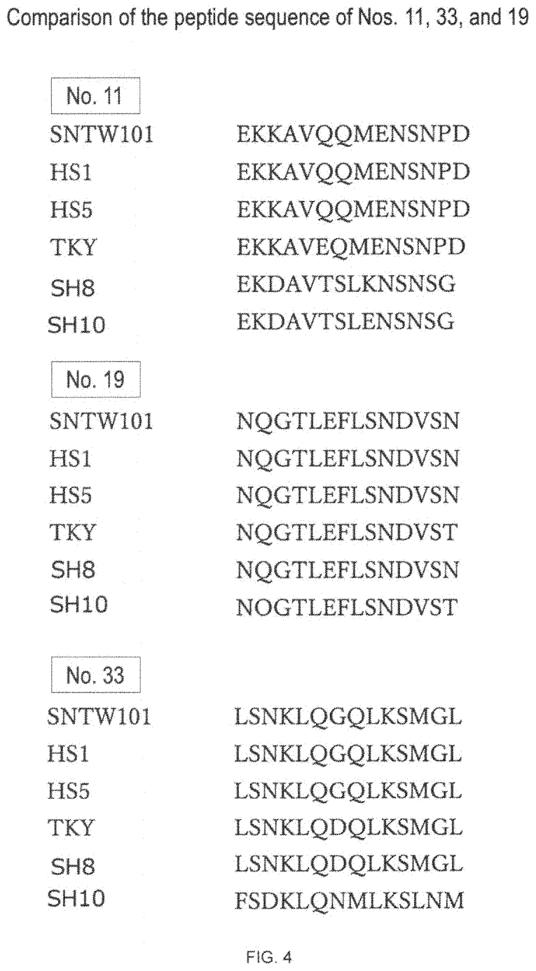

FIG. 5 shows the determination of H. suis by PCR. In photographs for H. pylori (SS1, TN2GF4, NCTC11637, ATCC43579, and RC-1 strains) and H. suis (SNTW101 and TKY strains), DNA was amplified by PCR using designed forward primer FW2, FW3, FW5, or VAC3624F (Non Patent Literature 7 described above) and reverse primer RV1, RV3, RV4, or VAC4041R (Non Patent Literature 7 described above), and the amplification product was confirmed by electrophoresis. Each lane depicts templated DNA prepared from 1) H. suis TKY, 2) H. suis SNTW101, 3) H. pylori SS1, 4) H. pylori TN2GF4, 5) H. pylori NCTC11637, 6) H. pylori ATCC43579, and 7) H. pylori RC-1 strains. The primer set used is shown below each photograph. For all the primer sets of the forward primer FW2, FW3, or FW5 and the reverse primer RV1, RV3, or RV4, the amplification product was confirmed only from DNA prepared from the gastric mucosa of an H. suis-infected mouse.

FIG. 6 is a graph showing results of measuring the binding titers of antibodies in serum obtained from A, an H. suis-infected human, B, an H. pylori-infected human, and C, a non-infected human against various peptides by ELISA. The abscissa depicts Nos of the peptides. The ordinate depicts absorbance at 450 nm. A mean from the experiment conducted in duplicate is shown. Synthesized 15 HsvA antigen peptides (Nos. 10 (SEQ ID NO: 14), 11 (SEQ ID NO: 3), 14 (SEQ ID NO: 20), 15 (SEQ ID NO: 16), 16 (SEQ ID NO: 12), 19 (SEQ ID NO: 7), 20 (SEQ ID NO: 18), 21 (SEQ ID NO: 15), 22 (SEQ ID NO: 19), 23 (SEQ ID NO: 13), 26 (SEQ ID NO: 21), 31 (SEQ ID NO: 22), 33 (SEQ ID NO: 9), 34 (SEQ ID NO: 17), and 35 (SEQ ID NO: 23)) specifically reacted with the serum of the subject infected with H. suis.

FIG. 7 is a graph showing results of measuring the binding titers of antibodies in the serum of 5 subjects against No. 11 peptide (SEQ ID NO: 3), No. 19 peptide (SEQ ID NO: 7), and No. 33 peptide (SEQ ID NO: 9) by ELISA. A and B show data from the serum of different H. suis-infected subjects, respectively. C and D show data from the serum of different H. pylori-infected subjects, respectively. E shows data from the serum of a non-infected subject. The abscissa depicts SEQ ID NOs of the antigen peptides and dilution ratios of the serum. The ordinate depicts absorbance at 450 nm. A mean from the experiment conducted in triplicate and standard deviation are shown. All the peptides strongly reacted with the H. suis-infected serum and weakly reacted with the H. pylori-infected or non-infected serum.

FIG. 8 is a graph showing results of measuring the binding titers of antibodies in the serum of 6 subjects (two H. suis-infected subjects, three H. pylori-infected subjects, and one non-infected subject) against No. 11 peptide (SEQ ID NOs: 3, 4, 5, 6, and 24), No. 19 peptide (SEQ ID NOs: 7, 8, and 25) and No. 33 peptide (SEQ ID NOs: 9, 10, 11, and 26) by ELISA. A and B show data from the serum of different H. suis-infected subjects, respectively. C, D and E show data from the serum of different H. pylori-infected subjects, respectively. F shows data from the serum of a non-infected subject. The abscissa depicts SEQ ID NOs of the antigen peptides and dilution ratios of the serum. The ordinate depicts absorbance at 450 nm. A mean from the experiment conducted in duplicate is shown. All the peptides strongly reacted with the H. suis-infected serum and weakly reacted with the H. pylori-infected or non-infected serum.

FIG. 9 is a graph showing results of measuring the binding titers of antibodies in the serum of 8 subjects (three H. suis-infected subjects, three H. pylori-infected subjects, and two non-infected subjects) against whole cells of an H. pylori TN2GF4 strain by ELISA. A and B show results obtained from serum diluted 600-fold and 1800-fold, respectively. The ordinate depicts absorbance at 450 nm. A mean from the experiment conducted in duplicate is shown. Only the H. pylori-infected serum strongly reacted with the bacterial cells of H. pylori.

FIG. 10 is a graph showing results of measuring the binding titers of the serum of mice infected with an H. suis TKY or H. suis SNTW101 strain against No. 11 peptide (SEQ ID NOs: 3, 4, 5, 6, and 24), No. 19 peptide (SEQ ID NOs: 7, 8, and 25), No. 33 peptide (SEQ ID NOs: 9, 10, 11, and 26) and whole cells of H. pylori TN2GF4 and SS1 strains by ELISA. The abscissa depicts the types of the peptides or the bacterial cells used in ELISA. A, B and C show data from the serum of different H. suis TKY strain-infected mice, respectively. D, E and F show data from the serum of different H. suis SNTW101 strain-infected mice, respectively. A and D show results obtained from serum diluted 20-fold. B and E show results obtained from serum diluted 100-fold. C and F show results obtained from serum diluted 400-fold. The abscissa depicts SEQ ID NOs of the antigen peptides and dilution ratios of the serum. The ordinate depicts absorbance at 450 nm. A mean from the experiment conducted in duplicate is shown. The H. suis-infected mouse serum strongly reacted with the H. pylori whole cells, but also strongly reacted with all the peptides of Nos. 11, 19, and 33.

FIG. 11 is a graph showing results of measuring the serum of mice infected with an H. suis TKY or H. pylori SS1 strain by sandwich ELISA. The abscissa depicts the types of peptides or bacterial cells used in sandwich ELISA. The peptides and the bacterial cells used are as follows: SEQ ID NO: 3 (peptide No. 11 (SNTW101; HS1; HS5), SEQ ID NO: 7 (peptide No. 19 (SNTW101; HS1; H55, SH8), and SEQ ID NO: 9 (peptide No. 33 (SNTW101; HS1; HS5). The ordinate depicts absorbance at 450 nm. A mean from the experiment conducted in triplicate and standard deviation are shown. Only the H. suis-infected mouse serum strongly reacted with all the peptides of Nos. 11, 19, and 33.

FIG. 12 is a graph showing results of ELISA about the binding of each rabbit antibody to various antigens (HsvA-derived peptides or H. pylori whole cells). The abscissa depicts various antigens (represented by the same numbers as in FIG. 10) immobilized on a solid phase. The ordinate depicts absorbance at 450 nm. A shows the results obtained using a rabbit antibody against No. 11 peptide (SEQ ID NO: 3) prepared in Example 2. B shows the results obtained using a rabbit antibody against No. 33 peptide (SEQ ID NO: 9) prepared in Example 2. C shows the results obtained using a rabbit antibody against No. 19 peptide (SEQ ID NO: 7) prepared in Example 2. A mean from the experiment conducted in triplicate and standard deviation are shown. The antibody against each peptide strongly reacted with the peptide, but weakly reacted with the H. pylori whole cells.

FIG. 13 shows immunohistochemical photographs obtained using a polyclonal antibody obtained by immunizing a rabbit with No. 11 peptide. A gastric tissue section of an H. suis-infected patient was reacted with a rabbit antibody against No. 11 peptide adjusted to 2 μg/mL with phosphate-buffered saline and then reacted with Alexa-Flour 488 anti-rabbit IgG diluted 400-fold with phosphate-buffered saline, and photographed under Leica confocal laser fluorescence microscope (TCS-SP5). Alexa-Flour 568 diluted 400-fold with phosphate-buffered saline was used in counterstaining. A is a photograph taken at a 200× magnification. B is a photograph of the boxed portion of A magnified 760 times. The arrows depict bacterial cells of H. suis.

FIG. 14 is a graph showing results of measuring the binding activity of antibodies in serum collected from patients infected with H. suis or H. pylori against each peptide or bacterial cells by ELISA. The values are indicated by mean±standard deviation (SD). A shows the binding of antibodies in serum collected from H. suis-infected patients (n=4) to each peptide. The ordinate depicts absorbance at 450 nm. The abscissa depicts SEQ ID NOs of peptides used in immobilization on a solid phase. B shows the binding of antibodies in serum collected from H. pylori-infected patients (n=5) to each peptide. The ordinate depicts absorbance at 450 nm. The abscissa depicts SEQ ID NOs of peptides used in immobilization on a solid phase. C shows the binding of antibodies in serum obtained from non-infected patients (n=3) to each peptide. The ordinate depicts absorbance at 450 nm. The abscissa depicts SEQ ID NOs of peptides used in immobilization on a solid phase. D shows the binding of antibodies contained in H. suis-infected patient-derived serum, H. pylori-infected patient-derived serum, and non-infected patient-derived serum diluted 600-fold or 1800-fold to H. pylori TN2GF4 (filled marks) or SS1 (open marks). The ordinate depicts absorbance at 450 nm. The abscissa depicts the attributions of the patients from which the serum used was derived (H. suis: H. suis-infected patient, H. pylori: H. pylori-infected patient, none: non-infected patient). 1:600 shown in the upper part represents that the patient serum was diluted 600-fold and used as a sample. 1:1800 represents that the patient serum was diluted 1800-fold and used as a sample. In both “1:600” and “1:1800”, the results indicated by filled marks on the left side depict binding to H. pylori TN2GF4, and the results indicated by open marks on the right side depict binding to H. pylori SS1. ***P<0.0001 (H. suis-infected serum vs. H. pylori-infected serum or non-infected serum).

FIG. 15 is a graph showing results of examining the specificity of an antibody against each of peptides of Nos. 61, 11, 33, 19, 10, and 16 for the corresponding antigen peptide. The upper part of each graph describes the type of the peptide as an antigen with which the antibody measured in this graph was obtained (e.g., “Ab. against SEQ ID NO: 91” means the antibody obtained with the peptide of SEQ ID NO: 91 as an antigen). The ordinate depicts absorbance at 450 nm. The abscissa depicts SEQ ID NOs of the peptides used in immobilization on a solid phase. The values are indicated by mean±standard deviation (SD).

FIG. 16 is a graph showing results of measuring the binding activity of anti-peptide rabbit polyclonal antibodies obtained in Example 2 against bacterial cells of each Helicobacter species by ELISA. The upper part of each graph describes the type of the peptide as an antigen with which the antibody measured in this graph was obtained (e.g., “Ab. against SEQ ID NO: 91” means the antibody obtained with the peptide of SEQ ID NO: 91 as an antigen). The ordinate depicts absorbance at 450 nm. The abscissa depicts Helicobacter strains used in immobilization on a solid phase. The values are indicated by mean±standard deviation (SD).

FIG. 17 shows photographs showing results of immunohistochemically staining (A) an H. suis SNTW101-infected mouse gastric section, (B) an enlarged view of the boxed portion of (A), and (C) a non-infected mouse gastric section using an anti-No. 16 peptide (SEQ ID NO: 12) antibody. The arrows depict stained bacterial cells.

### DESCRIPTION OF EMBODIMENTS

In one aspect, the present invention relates to a method for determining the presence of H. suis in a sample, comprising detecting hsvA gene or a portion thereof, HsvA protein or a portion thereof, or an antibody against the HsvA protein in the sample. Particularly, the present invention relates to a method for determining infection of a subject with H. suis, comprising detecting hsvA gene or a portion thereof, HsvA protein or a portion thereof, or an antibody against the HsvA protein in a sample derived from the subject. In the present method, the sample or the subject in which the hsvA gene or a portion thereof, the HsvA protein or a portion thereof, or the antibody against the HsvA protein has been detected is determined to have H. suis or to be infected with H. suis.

When the method of the present invention is performed by measuring the binding of a polynucleotide, a protein or a portion thereof, or an antibody or a portion thereof in the sample to a test reagent, whether or not to “have been detected” in the determination does not require the absolute presence or absence of detection and may be determined by comparison with other samples. Specifically, the presence or absence of detection may be determined from measurement values, rather than being based on ±detection results. Specifically, in the method of the present invention, the “detecting” step is interchangeable, if necessary, with “measuring”, and whether or not to “have been detected” may be determined by comparison with a negative control based on a measurement value of an intended substance. For example, even when the substance of interest is detected in a negative control, i.e., a sample containing no H. suis or a sample derived from a subject evidently having no H. suis infection, the substance detected in a very small amount is regarded as “having not been detected” in the method of the present invention as long as the measurement value of the subject is equivalent to that of the negative control. Thus, the sample or the subject is determined to have no H. suis or to be not infected with H. suis. On the other hand, when the measurement value of the subject is higher than that of the negative control, the substance is regarded as “having been detected”. Thus, the sample or the subject is determined to have H. suis or to be infected with H. suis. Accordingly, in the method of the present invention, slight measurement value found in a negative control is acceptable by the present invention.

Preferably, the method for determining the presence of H. suis or the method for determining infection of a subject with H. suis according to the present invention employs at least one member selected from the group consisting of (i) a nucleic acid molecule capable of specifically binding to hsvA gene, (ii) an HsvA antigen peptide, and (iii) an anti-HsvA antibody or an immunoreactive fragment thereof in a sample derived from the subject.

(Method Using Nucleic Acid Molecule Having hsvA Gene-Specific Nucleotide Sequence)

Whether or not a subject is infected with H. suis can be determined as the presence or absence of the infection by detecting hsvA gene present in a sample of the subject. The detection of H. suis using the nucleic acid molecule capable of specifically binding to hsvA gene can be performed by a method using hybridization; a method using FOR; or a method known as Invader® assay (see, for example, Kwiatkowski, R. W. et al., Mol. Diagn. (1999) 4: 353-364).

Examples of the method using hybridization can include methods such as Southern hybridization, Northern hybridization, dot hybridization, fluorescence in situ hybridization (FISH), DNA microarrays, and ASO. The method for determining the presence of H. suis in a sample according to the present invention may comprise, for example:

(a) contacting the sample with at least one nucleic acid molecule capable of specifically binding to hsvA gene;
(b) detecting binding of a polynucleotide in the sample to the nucleic acid molecule capable of specifically binding to hsvA gene; and
(c) determining the sample in which the binding has been detected, as a sample having H. suis, and determining the sample in which the binding has not been detected, as a sample having no H. suis, and thereby determining the presence of H. suis.

The method of the present invention may be, for example, a method for determining infection of a subject with H. suis, comprising:

(a) contacting a sample derived from the subject with at least one nucleic acid molecule capable of specifically binding to hsvA gene;
(b) detecting binding of a polynucleotide in the sample to the nucleic acid molecule capable of specifically binding to hsvA gene; and
(c) determining the subject from which the sample in which the binding has been detected is derived, to be infected with H. suis, and/or determining the sample in which the binding has not been measured or detected, as a sample having no H. suis.

The binding of a polynucleotide in the sample to the nucleic acid molecule capable of specifically binding to hsvA gene can be detected, for example, by labeling in advance the nucleic acid molecule capable of specifically binding to hsvA gene, contacting a sample derived from the subject with the nucleic acid molecule capable of specifically binding to hsvA gene, then performing washing, and detecting or measuring the label on the remaining nucleic acid molecule capable of specifically binding to hsvA gene. In this case, preferably, the sample derived from the subject is immobilized on a solid phase.

Examples of the method using PCR can include methods such as ARMS (amplification refractory mutation system), RT-PCR (reverse transcription-PCR), and nested PCR. The method for determining the presence of H. suis according to the present invention may comprise, for example, the step of:

(a) amplifying a portion of hsvA gene in a sample using a nucleic acid molecule capable of specifically binding to hsvA gene as a primer;
(b) detecting the amplified nucleic acid molecule; and
(c) determining the sample in which the amplified nucleic acid molecule has been detected, as a sample having H. suis, and determining the sample in which the amplified nucleic acid molecule has not been detected, as a sample having no H. suis, and thereby determining the presence of H. suis.

The present invention also relates to a method for determining H. suis infection, comprising:

(a) amplifying a portion of hsvA gene in a sample derived from a subject using a nucleic acid molecule capable of specifically binding to hsvA gene as a primer;
(b) detecting the amplified nucleic acid molecule; and
(c) determining the subject from which the sample in which the amplified nucleic acid molecule has been detected is derived, to be infected with H. suis, and determining the subject from which the sample in which the amplified nucleic acid molecule has not been detected is derived, to be not infected with H. suis.

In another aspect, the present invention relates to a method for determining H. suis infection, comprising:

(a) amplifying a portion of hsvA gene in a sample derived from a subject using a hsvA gene-specific nucleic acid molecule or the like as a primer;
(b) measuring a level of the amplified nucleic acid molecule; and
(c) determining the subject to be infected with H. suis when the measured level of the nucleic acid molecule is higher than that of the nucleic acid molecule measured for a negative control by a similar method, and determining the subject to be not infected with H. suis when the measured level of the nucleic acid molecule is equivalent to or lower than that of the nucleic acid molecule measured for a negative control by a similar method.

The amplification of a portion of the hsvA gene in the sample derived from the subject can be carried out through PCR reaction or the like using the sample derived from the subject as a template.

The amplified nucleic acid can be determined by dot blot hybridization, surface plasmon resonance (SPR) method, PCR-RFLP, in situ RT-PCR, PCR-SSO (sequence specific oligonucleotide), PCR-SSP, AMPFLP (amplifiable fragment length polymorphism), MVR-PCR, or PCR-SSCP (single strand conformation polymorphism).

The method for determining the presence of H. suis using the method known as Invader® assay may comprise, for example, the steps of:

(a) contacting a specimen with the following nucleic acids (i) and (ii) to form a triplex of DNA complementary to an allele probe:

(i) an allele-specific probe having an hsvA gene-specific nucleotide sequence or a sequence complementary to the sequence and a sequence complementary to a portion of a quenching probe (flap), and/or an allele-specific probe having an hsvA-specific nucleotide sequence or a sequence complementary to the sequence and a sequence complementary to a portion of a quenching probe (flap), and

(ii) an Invader probe having the hsvA gene-specific nucleotide sequence;

(b) contacting triplex-specific DNase with the nucleic acid specimen obtained by the step (a) to liberate the flap from the nucleic acid triplex;
(c) contacting the liberated flap with fluorescently labeled universal probes each having a sequence complementary to the flap and the quenching probe;
(d) contacting triplex-specific DNase with the nucleic acid specimen obtained by the step (c) to liberate the fluorescent label so that fluorescence is emitted;
(e) detecting the emitted fluorescence and thereby detecting the hsvA gene, wherein the sample in which the fluorescence emission has been detected is determined as a sample having H. suis, and the sample in which the fluorescence emission has not been detected is determined as a sample having no H. suis, thereby determining the presence of H. suis.

(Method Using Detection of HsvA Protein-Specific Antibody)

Whether or not a subject is infected with H. suis can be determined by confirming the presence of an antibody against an H. suis-derived protein (particularly, HsvA protein) produced by the own immune system of the subject. The detection of H. suis using the HsvA antigen peptide, or the anti-HsvA antibody or the immunoreactive fragment thereof can be performed by, for example, labeled immunoassay such as enzyme immunoassay (EIA), simple EIA, enzyme-linked immunosorbent assay (ELISA), radioimmunoassay (RIA), or fluorescence immunoassay (FIA); immunoblotting such as Western blotting; immunochromatography such as metal colloid agglutination method; chromatography such as ion-exchange chromatography or affinity chromatography; turbidimetric immunoassay (TIA); nanofluidic proteomic immunoassay (NIA); colorimetric method; latex immunoassay (LIA); counting immunoassay (CIA); chemiluminescent (enzyme) immunoassay (CLIA and CLEIA); precipitation reaction method; surface plasmon resonance (SPR) method; resonant mirror detector (RMD) method; or comparison interferometry.

In one aspect, the present invention relates to a method for determining H. suis infection, comprising:

(a) contacting a sample derived from a subject with the HsvA antigen of the present invention;
(b) detecting an antibody bound with the peptide, in the blood sample; and
(c) determining the subject in which the antibody bound with the peptide has been detected, to be infected with H. suis, and determining the subject in which the antibody bound with the peptide has not been detected, to be not infected with H. suis.

In another aspect, the present invention relates to a method for determining H. suis infection, comprising:

(a) contacting a sample derived from a subject with the HsvA antigen peptide of the present invention;
(b) measuring a level of an antibody bound with the peptide, in the blood sample; and
(c) determining the subject to be infected with H. suis when the measured level of the antibody is higher than that of the antibody measured for a negative control by a similar method, and determining the subject to be not infected with H. suis when the measured level of the antibody is equivalent to or lower than that of the antibody measured for a negative control by a similar method.

Whether or not the antibody in the sample derived from the subject is bound with the peptide can be confirmed, for example, by contacting the sample derived from the subject with the HsvA antigen peptide of the present invention, then washing the reaction solution to remove an unbound antibody, contacting a labeled anti-Ig antibody therewith so that a subject-derived antibody bound with the HsvA antigen peptide binds to the anti-Ig antibody, washing the reaction solution to remove an unbound antibody, then detecting the label on the anti-Ig antibody, and determining binding between the peptide and the antibody in the sample derived from the subject when the label on the anti-Ig antibody is detected. The binding level between the peptide and the antibody in the sample derived from the subject may be measured by measuring the label level of the anti-Ig antibody. In using the binding level, the amount of the antibody can be calculated from a measurement value by preparing a calibration curve using a standard solution having an abundance known in advance. Alternatively, the binding between the peptide and the antibody in the sample derived from the subject may be detected or measured using a surface plasmon resonance sensor.

(Diagnosis of H. suis Infection Using Detection of HsvA Antigen Peptide)

Whether or not a subject is infected with H. suis can be determined by confirming the presence of an H. suis-derived protein (particularly, HsvA protein) in a sample derived from the subject. The detection of H. suis using the HsvA protein can be performed by a method similar to the aforementioned method using the detection of the HsvA protein-specific antibody.

The method for determining the presence of H. suis of the present invention may comprise, for example:

(a) contacting a sample with a labeled anti-HsvA antibody or immunoreactive fragment thereof;
(b) detecting the label on a bound antibody; and
(c) determining the sample in which the label has been detected, as a sample having H. suis, and determining the sample in which the binding has not been detected, as a sample having no H. suis, and thereby determining the presence of H. suis.

The method for determining infection of a subject with H. suis of the present invention may comprise, for example:

(a) contacting a sample derived from the subject with a labeled anti-HsvA antibody or immunoreactive fragment thereof;
(b) detecting the label on a bound antibody; and
(c) determining the subject from which the sample in which the label has been detected is derived, to be infected with H. suis, and/or determining the subject from which the sample in which the label has not been detected is derived, to be not infected with H. suis.

Alternatively, the method for determining the presence of H. suis of the present invention may comprise, for example:

(a) contacting a sample with an anti-HsvA antibody or an immunoreactive fragment thereof;
(b) contacting the sample obtained by the step (a) with a labeled anti-HsvA antibody or immunoreactive fragment thereof which is different from said anti-HsvA antibody or said immunoreactive fragment thereof;
(c) measuring or detecting the label on a bound antibody; and
(d) determining the sample in which the binding has been measured or detected, as a sample having H. suis, and determining the sample in which the binding has not been measured or detected, as a sample having no H. suis, and thereby determining the presence of H. suis.

The method for determining infection of a subject with H. suis of the present invention may comprise, for example:

(a) contacting a sample derived from the subject with an anti-HsvA antibody or an immunoreactive fragment thereof;
(b) contacting the sample obtained by the step (a) with a labeled anti-HsvA antibody or immunoreactive fragment thereof which is different from said anti-HsvA antibody or said immunoreactive fragment thereof;
(c) measuring or detecting the label on a bound antibody; and
(d) determining the subject from which the sample in which the binding has been measured or detected is derived, to be infected with H. suis, and/or determining the subject from which the sample in which the binding has not been measured or detected is derived, to be not infected with H. suis.

The labeling of the antibody or the fragment thereof can be performed by a general method in the art. For example, in fluorescently labeling a protein or a peptide, the protein or the peptide is washed with a phosphate buffer solution. Then, a dye prepared with DMSO, a buffer solution, or the like is added thereto and mixed. Then, the mixture can be left standing at room temperature for 10 minutes for binding. Alternatively, the labeling may be performed using a commercially available labeling kit such as a biotin labeling kit (Biotin Labeling Kit-NH2 or Biotin Labeling Kit-SH; Dojindo Laboratories), an alkaline phosphatase labeling kit (Alkaline Phosphatase Labeling Kit-NH2 or Alkaline Phosphatase Labeling Kit-SH; Dojindo Laboratories), a peroxidase labeling kit (Peroxidase Labeling Kit-NH2 or Peroxidase Labeling Kit-NH2; Dojindo Laboratories), a phycobiliprotein labeling kit (Allophycocyanin Labeling Kit-NH2, Allophycocyanin Labeling Kit-SH, B-Phycoerythrin Labeling Kit-NH2, B-Phycoerythrin Labeling Kit-SH, R-Phycoerythrin Labeling Kit-NH2, or R-Phycoerythrin Labeling Kit-SH; Dojindo Laboratories), a fluorescent labeling kit (Fluorescein Labeling Kit-NH2, HiLyte Fluor® 555 Labeling Kit-NH2, or HiLyte Fluor® 647 Labeling Kit-NH2; Dojindo Laboratories), DyLight 547, DyLight 647 (Techno Chemical Corp.), Zenon® Alexa Fluor® antibody labeling kit or Qdot® antibody labeling kit (Invitrogen Corp.), EZ-Label Protein Labeling Kit (Funakoshi Co., Ltd.). The labeled antibody or fragment thereof can be appropriately detected using an instrument suitable for the label.

H. suis infects a wide range of mammals. Therefore, the subject in the method for determining the presence of H. suis and the method for determining infection with H. suis described herein can be a mammal such as a human, a monkey, a pig, a cat, a dog, a rabbit, or sheep and is preferably a human, more preferably a human patient with gastritis, chronic gastritis, nodular gastritis, gastric MALT lymphoma, diffuse large B-cell lymphoma, stomach cancer, gastric or duodenal ulcer, idiopathic thrombocytopenic purpura, or functional dyspepsia. The method of the present invention can be performed qualitatively, quantitatively or semi-quantitatively.

For example, a tissue specimen collected from a subject by biopsy or a liquid collected from a subject can be used as the sample in the method for determining the presence of H. suis described herein. The sample is not particularly limited as long as the sample can be targeted by the method of the present invention. Examples thereof can include tissues, blood, plasma, serum, lymph, urine, feces, serous fluid, spinal fluid, joint fluid, aqueous humor, tears, saliva and fractionated or treated products thereof. When a nucleic acid is to be detected in the method for determining the presence of H. suis described herein, the sample is preferably a tissue (particularly, a gastric tissue (gastric biopsy sample)) or feces. When an antibody is to be detected in the method for determining the presence of H. suis described herein, the sample is preferably blood, plasma, serum, lymph, or urine.

The peptide, the nucleic acid molecule, and the antibody or the immunoreactive fragment thereof of the present invention can be appropriately prepared by methods well known to those skilled in the art with reference to the disclosure herein. The composition and the kit of the present invention can be appropriately produced by methods well known in the technical field.

The nucleic acid molecule and the antibody or the immunoreactive fragment thereof of the present invention specifically binds to H. suis. Therefore, for example, siRNA, antisense DNA or RNA, a neutralizing antibody or an immunoreactive fragment thereof, or a non-neutralizing antibody can be selected and thereby used as a pharmaceutical composition for removing H. suis from the body of a subject infected with H. suis, or for defending against H. suis infection. For example, the nucleic acid molecule and the antibody or the immunoreactive fragment thereof of the present invention can be purified, if necessary, then formulated according to a routine method, and thereby used as a pharmaceutical composition for treatment or prevention of a disease or disorder involving H. suis infection contributing to its development or exacerbation. The present invention also includes use of the nucleic acid molecule and the antibody or the immunoreactive fragment thereof of the present invention for producing an H. suis removing agent, or a therapeutic drug or a prophylactic drug for a disease or disorder involving H. suis infection contributing to its development or exacerbation. Alternatively, the present invention includes use of the nucleic acid molecule and the antibody or the immunoreactive fragment thereof of the present invention for removal of an H. suis, or a treatment or prevention of a disease or disorder involving H. suis infection contributing to its development or exacerbation. The present invention further relates to a method for removing H. suis, or a method for treating or preventing a disease or disorder involving H. suis infection contributing to its development or exacerbation, comprising administering the antibody of the present invention or the immunoreactive fragment thereof. The disease or the disorder involving H. suis infection contributing to its development or exacerbation herein includes gastritis, chronic gastritis, nodular gastritis, gastric MALT lymphoma, diffuse large B-cell lymphoma, stomach cancer, gastric or duodenal ulcer, idiopathic thrombocytopenic purpura, and functional dyspepsia. In the description above, “removal of H. suis” means that H. suis is removed from the body of a subject infected with H. suis. For use as a pharmaceutical composition, preferably, the antibody is humanized or a complete human antibody.

The pharmaceutical composition of the present invention may adopt any oral or parenteral preparation as long as the preparation can be administered to a patient. Examples of the composition for parenteral administration can include eye drops, injections, nasal drops, suppositories, patches, and ointments. An injection is preferred. Examples of the dosage form of the pharmaceutical composition of the present invention can include liquid preparations and freeze-dried preparations. For use as an injection, the pharmaceutical composition of the present invention can be supplemented, if necessary, with additives including solubilizers such as propylene glycol and ethylenediamine, buffers such as phosphate, tonicity agents such as sodium chloride and glycerin, stabilizers such as sulfite, preservatives such as phenol, and soothing agents such as lidocaine (see “Japanese Pharmaceutical Excipients Directory” Yakuji Nippo, Ltd. and “Handbook of Pharmaceutical Excipients Fifth Edition” APhA Publications). For use of the pharmaceutical composition of the present invention as an injection, examples of the storage container can include ampules, vials, prefilled syringes, pen-shaped cartridges for syringes, and bags for intravenous drips.

The pharmaceutical composition (therapeutic drug or prophylactic drug) of the present invention can be used as, for example, an injection which encompasses dosage forms such as intravenous injections, subcutaneous injections, intracutaneous injections, intramuscular injections, intravitreal injections, and drip injections. Such an injection can be prepared according to a known method, for example, by dissolving, suspending or emulsifying the antibody, etc. in a sterile aqueous or oily liquid usually used in injections. For example, physiological saline or an isotonic liquid containing glucose, sucrose, mannitol, or other pharmaceutical adjuvants can be used as the injectable aqueous liquid and can be used in combination with an appropriate solubilizer, for example, an alcohol (e.g., ethanol), a polyalcohol (e.g., propylene glycol and polyethylene glycol), or a nonionic surfactant [e.g., polysorbate 80, polysorbate 20, and HCO-50 (polyoxyethylene (50 mol) adduct of hydrogenated castor oil)]. For example, sesame oil or soybean oil can be used as the oily liquid and can be used in combination with a solubilizer such as benzyl benzoate or benzyl alcohol. The prepared injection liquid is filled into an appropriate ampule, vial, or syringe. Alternatively, an appropriate excipient may be added to the antibody of the present invention or the immunoreactive fragment thereof to prepare a freeze-dried preparation, which can be dissolved in injectable water, physiological saline, or the like in use and formulated as an injection liquid. In general, the oral administration of a protein such as an antibody is difficult because the protein is degraded by a digestive organ. However, the oral administration may be possible by making the best use of an antibody fragment or a modified antibody fragment and a dosage form. Examples of the preparation for oral administration can include capsules, tablets, syrups, and granules.

The pharmaceutical composition of the present invention is suitably prepared into a dosage form in a dosage unit adaptable to the amount of the active component administered. Examples of such a dosage form in a dosage unit include injections (ampules, vials, and prefilled syringes). Usually, 5 to 500 mg, 5 to 100 mg, or 10 to 250 mg of the antibody of the present invention or the immunoreactive fragment thereof may be contained per dosage unit of the dosage form.

The pharmaceutical composition (therapeutic drug or prophylactic drug) of the present invention may be administered locally or systemically. The administration method is not particularly limited, and the pharmaceutical composition is administered parenterally or orally as mentioned above.

Examples of the parenteral administration route include intraocular administration, subcutaneous administration, intraperitoneal administration, injection or intravenous drips into blood (intravenous or intraarterial) or spinal fluid. Administration into blood is preferred. The pharmaceutical composition (therapeutic drug or prophylactic drug) of the present invention may be temporarily administered or may be continuously or intermittently administered. The administration may be, for example, continuous administration for 1 minute to 2 weeks.

The amount of the pharmaceutical composition of the present invention administered is not particularly limited as long as the amount produces the desired therapeutic effect or prophylactic effect. The amount of the pharmaceutical composition administered can be appropriately determined depending on symptoms, sex, age, etc. For example, the amount of the pharmaceutical composition of the present invention administered can be determined using, as an index, a therapeutic effect or a prophylactic effect on the disease or the disorder involving cAMP contributing to its development or exacerbation. For use in, for example, prevention and/or treatment of a patient with the disease or the disorder involving cAMP contributing to its development or exacerbation, the pharmaceutical composition of the present invention is conveniently administered approximately one to ten times a month, preferably approximately one to five times a month, by intravenous injection at usually approximately 0.01 to 20 mg/kg body weight, preferably approximately 0.1 to 10 mg/kg body weight, more preferably approximately 0.1 to 5 mg/kg body weight, in terms of a single dose of the active ingredient. For other parenteral administration and oral administration approaches, an amount conforming thereto can be administered. Particularly, for severe symptoms, the amount or the number of doses may be increased according to the symptoms.

### EXAMPLES

Hereinafter, the present invention will be specifically described with reference to Examples. However, the present invention is not limited thereby. All literatures cited herein are incorporated herein by reference in their entirety.

### (Example 1) Detection by PCR

(1) Preparation of DNA

(1)-1. H. pylori

Eleven strains (SS1, TN2GF4, RC-1, ATCC43579, NCTC11637, TK1029, TK1081, TY1289, and TY281) were used as H. pylori. A bacterial liquid preserved at −80° C. was applied to Nissui Plate/Helicobacter Agar medium (Nissui Pharmaceutical Co., Ltd., Tokyo, Japan) and cultured at 37° C. for 3 days under microaerophilic conditions (5% 02, 10% CO₂, 85% N₂, humidity of 100%). The resulting colonies were suspended in sterile distilled water, treated at 95° C. for 5 minutes, and then centrifuged. The supernatant was used as crude purified chromosomal DNA. The crude purified chromosomal DNA was extracted with an equal amount of phenol:chloroform:isoamyl alcohol (25:24:1), then purified by ethanol precipitation, and dissolved in a small amount of sterile distilled water. The DNA concentration of the final product was determined according to the following expression by the measurement of absorbance at 260 nm (A260).

DNA concentration (μg/mL)=A260×50 (optical path length: 10 mm)

(1)-2. H. suis

Two strains (TKY and SNTW101) were used as H. suis. A female C57BL/6 mouse (Charles River Laboratories Japan, Inc., Yokohama, Japan) infected with H. suis was anatomized, and the greater curvature of the stomach was cut open. The contents were washed with PBS, and the mucosa was scraped using a glass slide. The mucosa was rubbed between opaque glass portions of two glass slides to prepare a suspension. DNA of the mouse gastric mucosa containing the bacterium was prepared using DNeasy Blood & Tissue kit (Qiagen N.V., Hilden, Germany). The DNA concentration of the final product was determined according to the following expression by the measurement of absorbance at 260 nm (A260).

DNA concentration (μg/mL)=A260×50 (optical path length: 10 mm)

(1)-3. H. felis, H. mustelae, H. Heilmannii s.s. (H. heilmannii Sensu Stricto), H. baculiformis, H. Bizzozeronii, H. Cynogastricus, and H. salmonis

A bacterial liquid preserved at −80° C. was suspended in sterile distilled water, treated at 95° C. for 5 minutes, and then centrifuged. The supernatant was used as crude purified chromosomal DNA. The crude purified chromosomal DNA was extracted with an equal amount of phenol:chloroform:isoamyl alcohol (25:24:1), then purified by ethanol precipitation, and dissolved in a small amount of sterile distilled water.

(2) PCR

(2)-1. PCR

A reaction solution (254 in total) having the following composition was subjected to PCR reaction in duplicate using Dream Taq DNA Polymerase (Thermo Fisher Scientific Inc.): 2.54 of a 10×buffer solution, 0.54 of 10 mM dNTP mix, 14 of a forward (FW) primer (5 μM), 14 of a reverse (RV) primer, 1 μL of template DNA (1 ng and 10 ng of each H. pylori (SS1, TN2GF4, RC-1, ATCC43579, NCTC11637, TK1029, TK1081, TY1289, and TY281 strains) DNA, and 10⁻³ ng, 10⁻² ng, 10⁻¹ ng, 1 ng, 10 ng, and 100 ng of each DNA prepared from the gastric biopsy of H. suis (SNTW101 and TKY strains)-infected mice), 0.25 μL of DNA polymerase, and 16.75 μL of sterile distilled water. The PCR reaction conditions was holding at 95° C. for 1 minute, then 35 cycles of 95° C. for 30 seconds, 55° C. for 30 seconds and 72° C. for 1 minute, and then keeping at 72° C. for 5 minutes.

Primers were designed from regions having high identity among H. suis strains and lacking identity to other bacterial species by comparing the H. suis hsvA gene with an autotransporter protein similar to VacA present in H. felis, H. bizzozeronii, and H. pylori. The designed primers were as follows.

| (Forward primer) | |
|---|---|
| (SEQ ID NO: 51) | |
| HSVANOFW-2: | AGAAACGAGATTACAGGAAG |
| (SEQ ID NO: 52) | |
| HSVANOFW-3: | AGGAGCAAGTTTTGTAGCAG |
| (SEQ ID NO: 53) | |
| HSVANOFW-5: | AAAATGCGACTGATTGGATG |
| (Reverse primer) | |
| (SEQ ID NO: 72) | |
| HSVANORV-1: | ATCGAAATAAGCGAACCTCA |
| (SEQ ID NO: 73) | |
| HSVANORV-3: | TTGAAAGCTTAGCTAAACGG |
| (SEQ ID NO: 74) | |
| HSVANORV4: | TGGTATTGCTGGTTAAGAGG |

The following primers were used as primers amplifying the H. pylori vacA gene (Matsui H et al., Helicobacter. (2014) 19 (4): 260-71).

| (SEQ ID NO: 84) | |
|---|---|
| VAC3624F (forward): | GAGCGAGCTATGGTTATGAC |
| (SEQ ID NO: 85) | |
| VAC4041R (reverse): | CATTCCTAAATTGGAAGCGAA |

104 of each obtained amplification product was electrophoresed on 1.5% agarose gel and stained with ethidium bromide to confirm an amplified band.

(2)-2. Real-Time PCR

A double quencher probe (PrimeTime® qPCR Probes) having the sequence given below was synthesized by outsourcing to Integrated DNA Technologies, Inc. (IDT). Since the primers FW5 and RW3 mentioned above have high identity among H. suis strains, new primers were designed from the same regions.

Probe:
(SEQ ID NO: 75)
FAM/TGTACACAC/ZEN/CAAACAGATGAGCCGT/3IABkFQ
Forward:
(SEQ ID NO: 47)
GATGGGCGCTTCTGGTTTA
Reverse:
(SEQ ID NO: 65)
CTGGTAATGCATCATTAGAAGCAAA

PCR reaction was performed under the following conditions using the probe (final concentration: 0.25 μM), the primers (final concentration: 0.5 μM), and PCR master mix: PrimeTime Gene Expression Master Mix (Integrated DNA Technologies, Inc. (IDT)) as well as QuantStudio (Applied Biosystems, Inc.) according to the IDT attached protocol.

95° C. for 3 minutes, 1 cycle
95° C. for 15 seconds—60° C. for 1 minute, 40 cycles

(3) Results

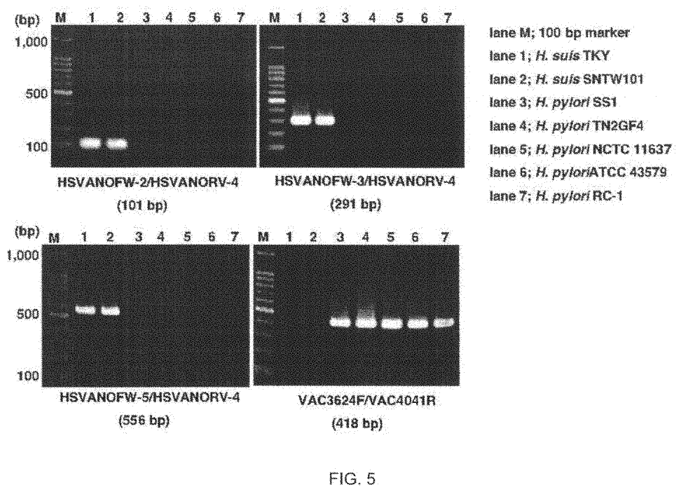

A calibration curve was prepared beforehand using plasmids into which a target region was cloned. As a result, favorable amplification efficiency and quantitativeness were exhibited from 10² copies to 10⁷ copies (not shown). Results of PCR using H. pylori DNA and gastric biopsied tissue DNA of H. suis-infected mouse are shown in FIG. 5. All the combinations of designed primers HSVANOFW-2/HSVANORV-4, HSVANOFW-3/HSVANORV-4, and HSVANOFW-5/HSVANORV-4 specifically amplified H. suis DNA and did not amplify H. pylori DNA. On the other hand, primers designed for H. pylori vacA amplified only H. pylori DNA and did not amplify H. suis DNA.

Results of quantitative PCR using gastric biopsied tissue DNA of an H. suis-infected mouse showed favorable amplification efficiency in amounts of DNA from 100 ng to 10⁻² ng (not shown). 6.9×10⁵ copies per 10 ng of DNA from the H. suis SNTW101 strain, 3.7×10⁵ copies per 10 ng of DNA from the H. suis TKY strain, 1.5×10³ copies per 10 ng of DNA from the H. suis SH8 strain, and 4.2×10³ copies per 10 ng of DNA from the H. suis SH10 strain were obtained. On the other hand, no amplification was observed in H. pylori DNA (SS1, TN2GF4, RC-1, ATCC43579, NCTC11637, TK1029, TK1081, TY1289, and TY281 strains). Likewise, H. felis, H. mustelae, H. heilmannii s.s. ASB1.4, H. baculiformis, H. bizzozeronii, H. cynogastricus, or H. salmonis chromosomal DNA used as a template was not amplified. No amplification was found for H. pylori (SS1, TN2GF4, NCTC11637, ATCC43579, RC-1, TK1029, TK1081, TY1289, and TY281 strains), H. felis, H. mustelae, H. heilmannii sensu stricto (H. heilmannii s.s. ASB1.4), H. baculiformis, H. bizzozeronii, H. cynogastricus, and H. salmonis. Only the H. suis-infected mouse-derived gastric mucosa DNA was amplified.

These results demonstrated that all the designed primer sets enable H. suis infection to be diagnosed by discrimination from H. pylori, H. felis, H. mustelae, H. heilmannii s.s. ASB1.4, H. baculiformis, H. bizzozeronii, H. cynogastricus, and H. salmonis.

### (Example 2) Peptide Synthesis and Antibody Preparation

H. suis was successfully detected specifically by PCR. Therefore, in consideration of the possibility that HsvA was expressed, a study was conducted on whether to be able to diagnose H. suis infection by targeting HsvA.

(1) Peptide Synthesis

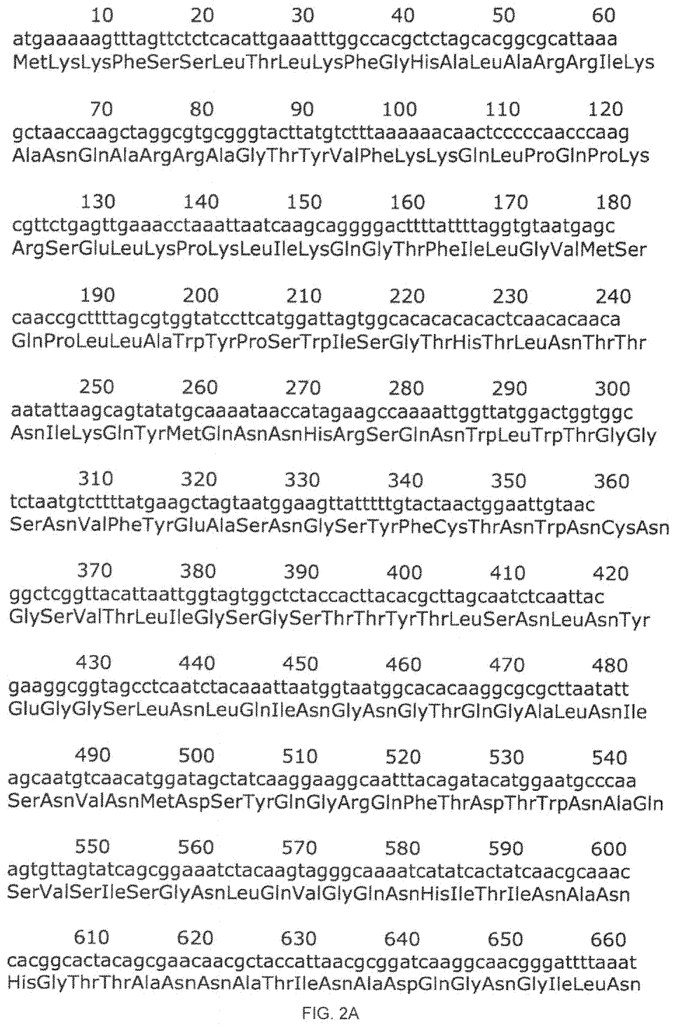

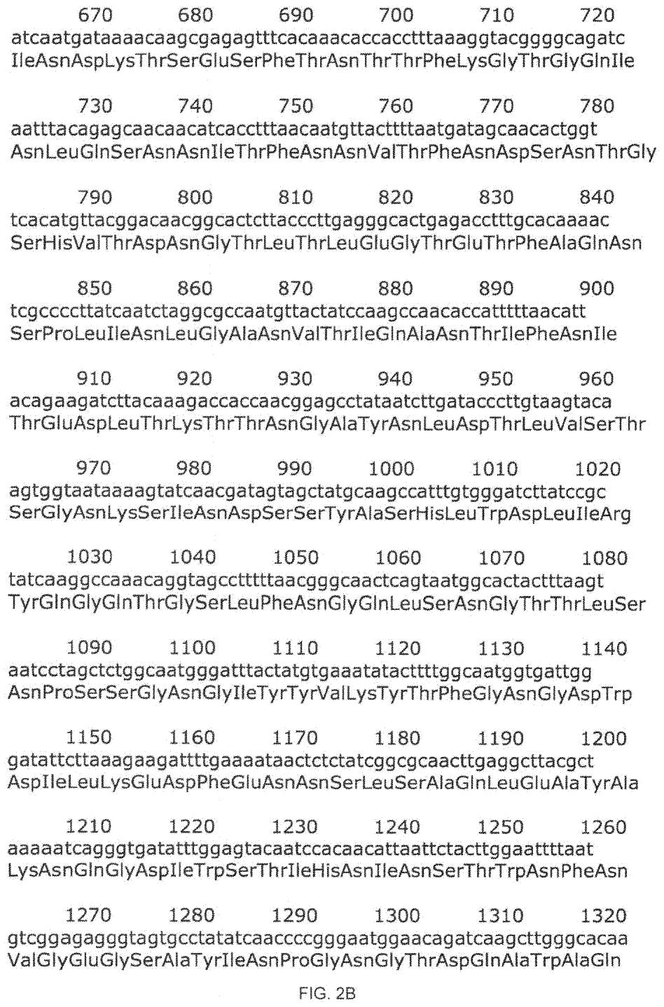

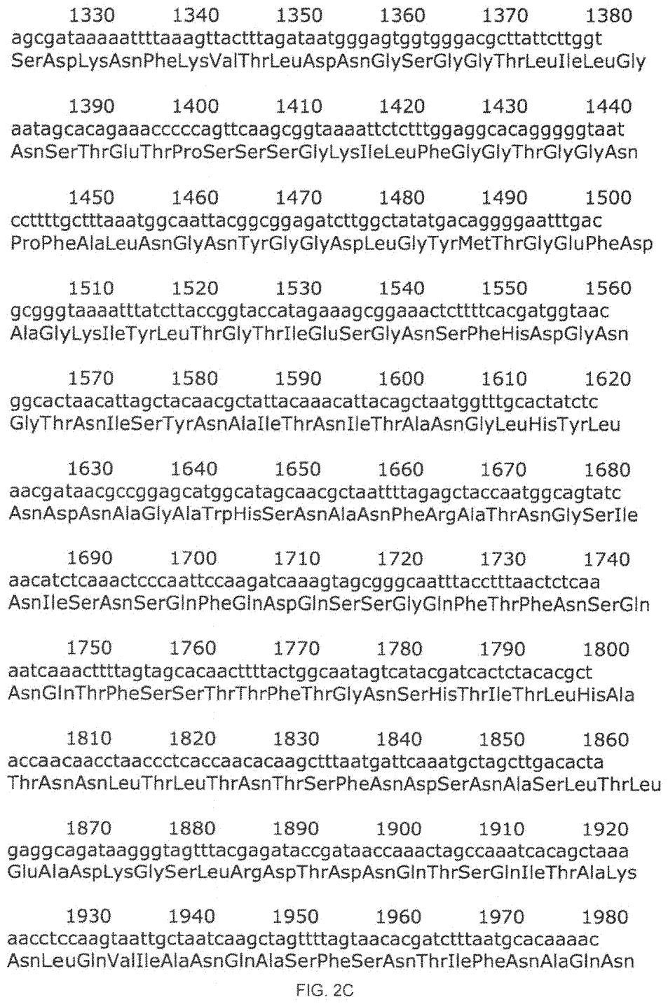

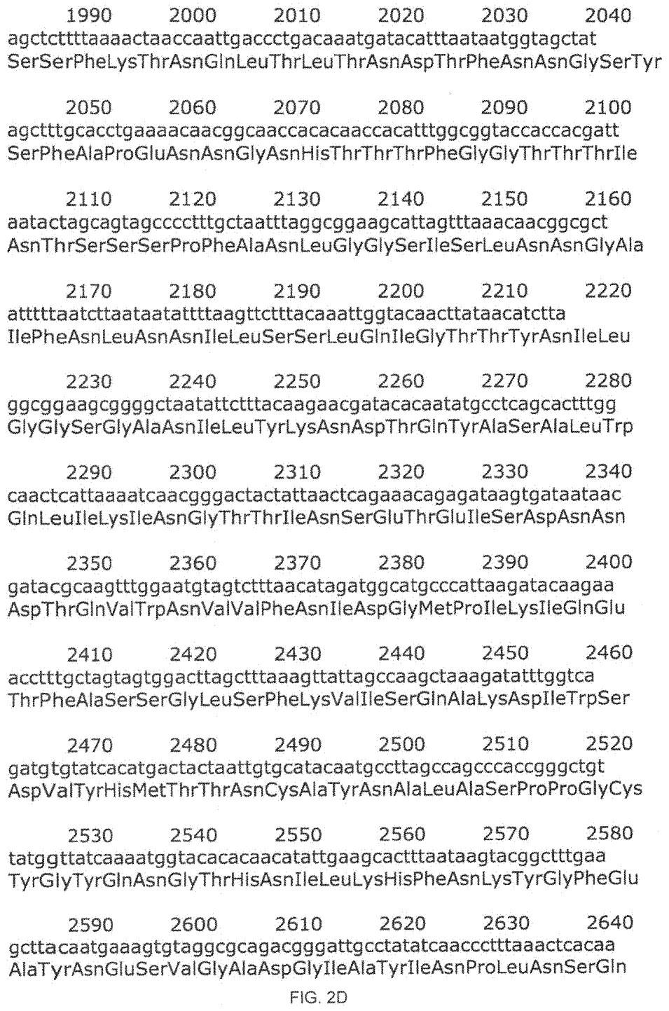

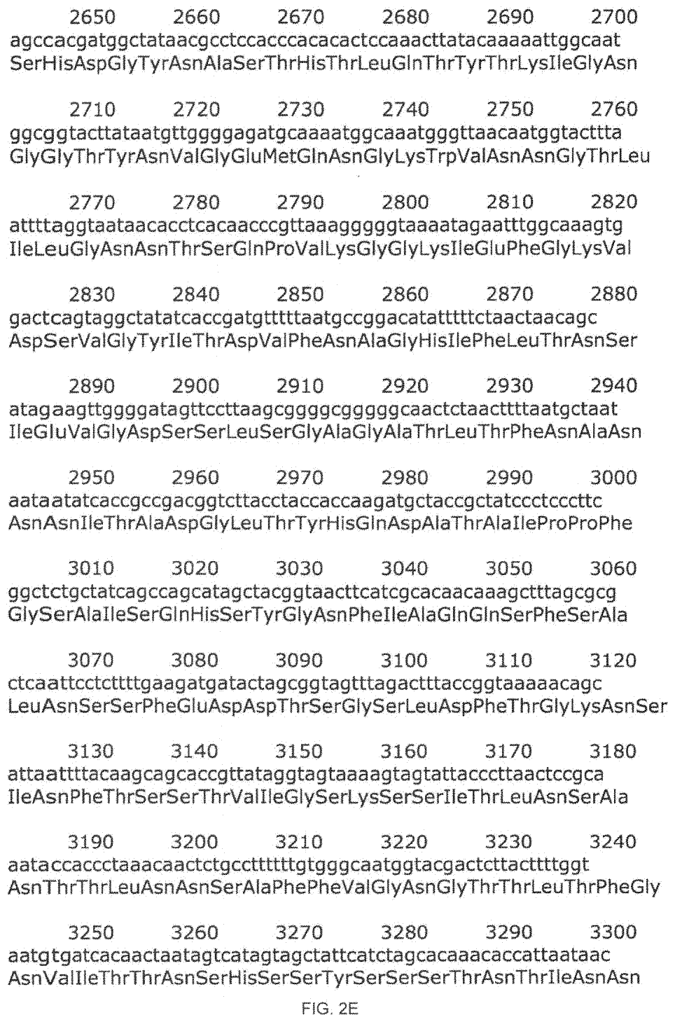

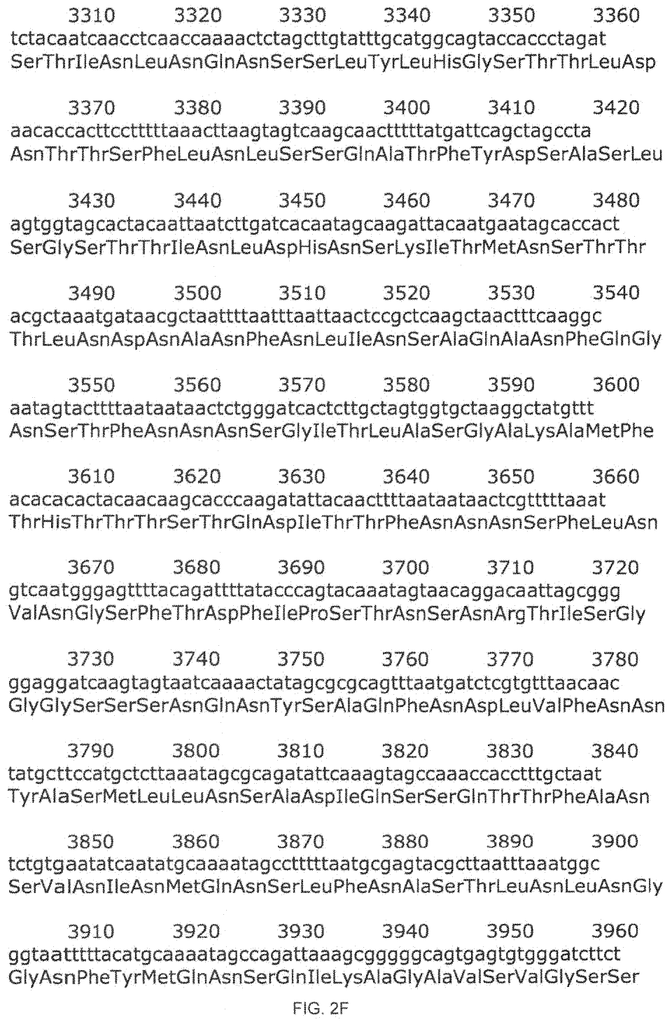

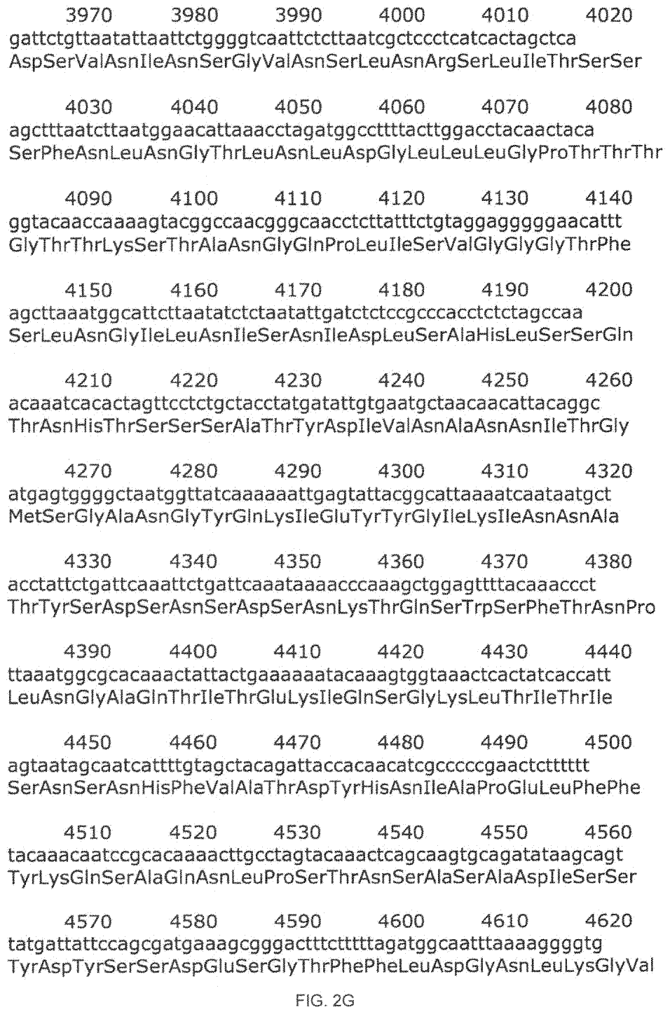

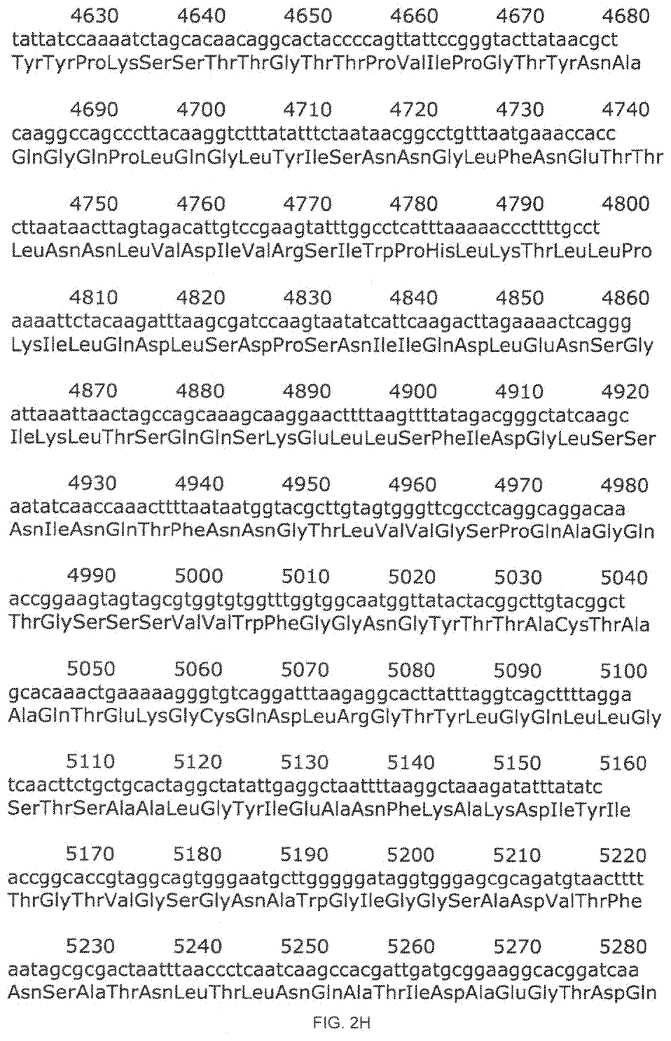

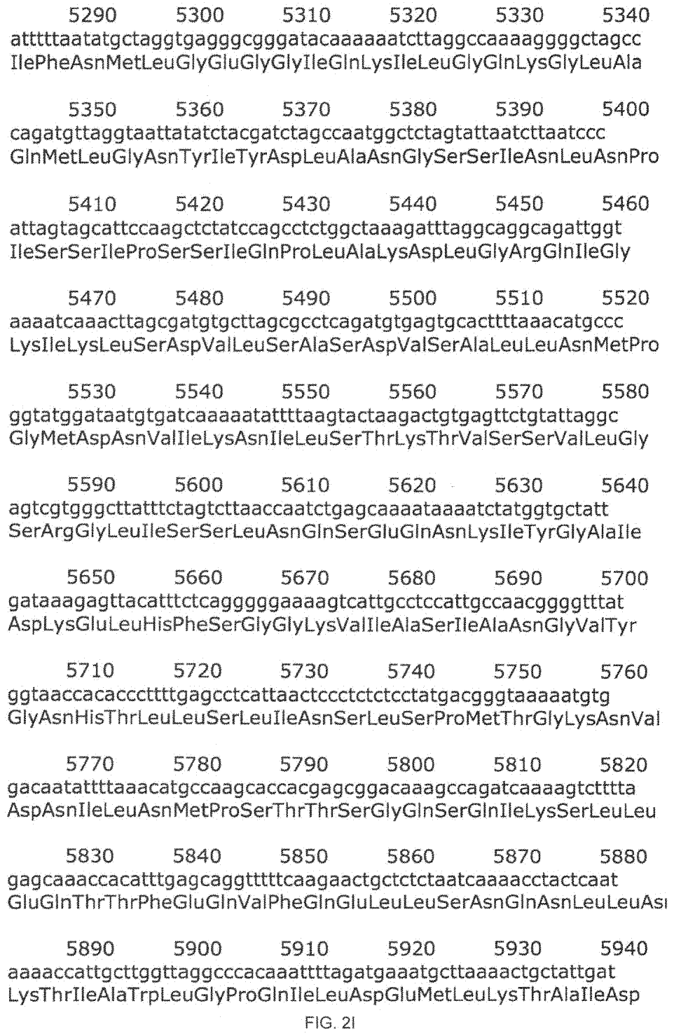

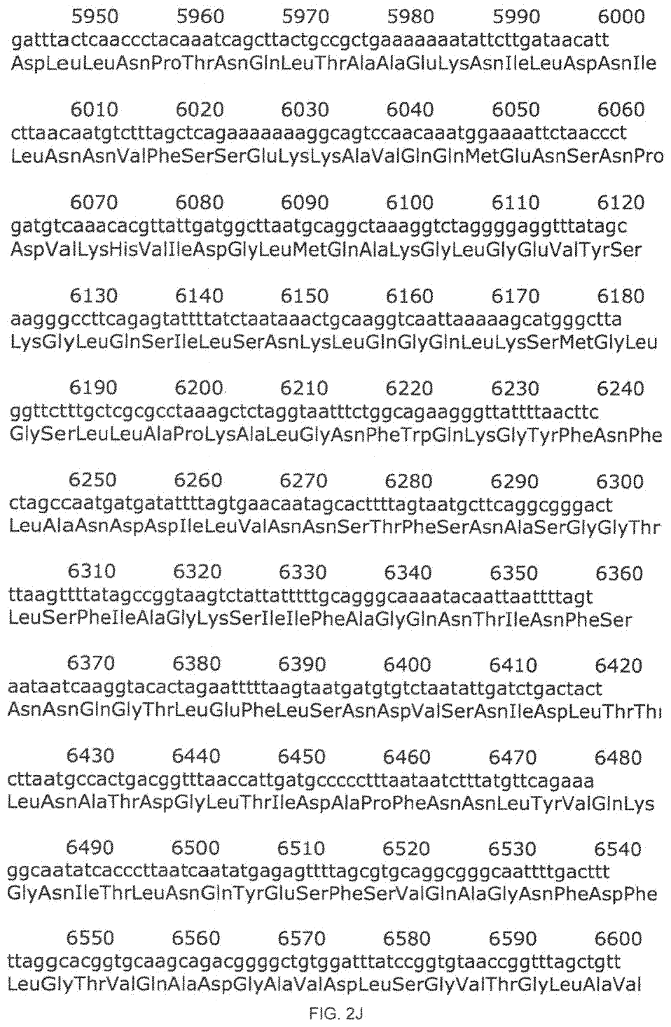

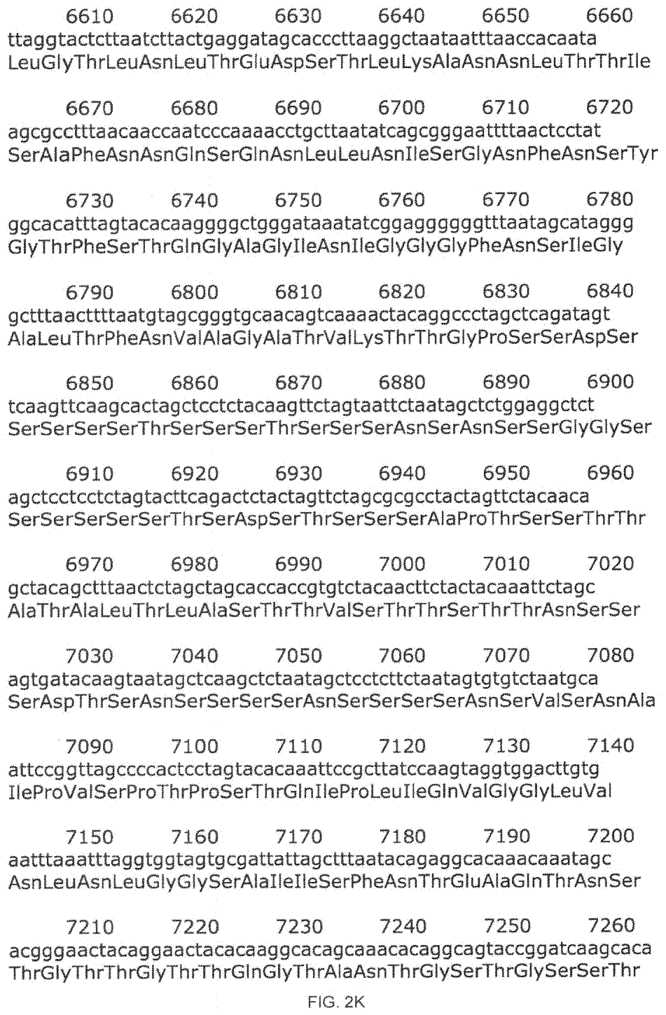

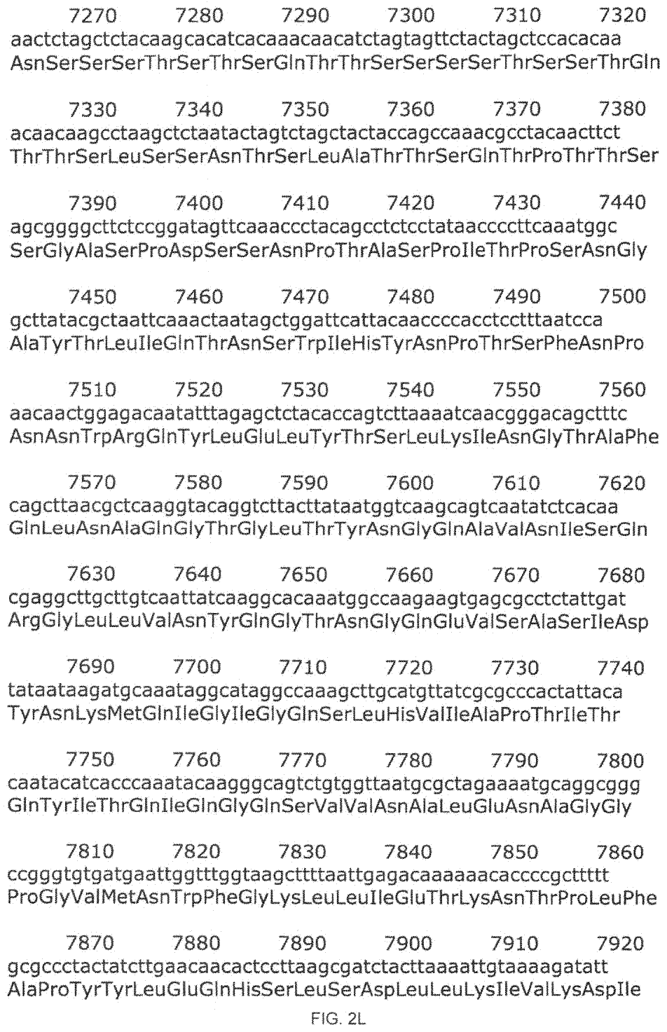

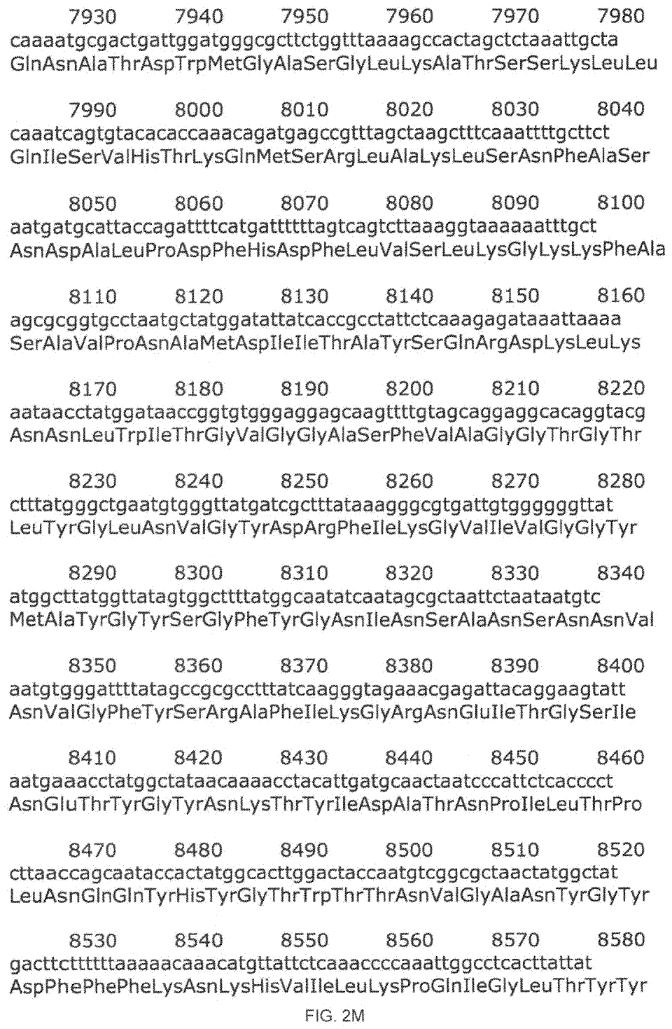

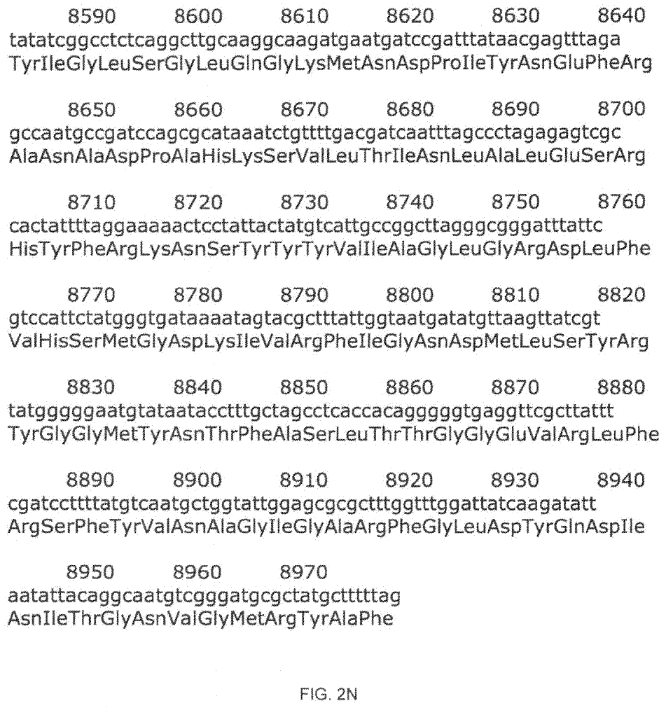

From the putative amino acid sequence of HsvA (SEQ ID NO: 2; FIG. 2), presumably antigenic peptide sequences were selected and synthesized based on the specificity of the bacterial species and conservation among strains.

(SEQ ID NO: 3)
No. 11: EKKAVQQMENSNPD
(SEQ ID NO: 4)
No. 11 (TKY): EKKAVEQMENSNPD
(SEQ ID NO: 5)
No. 11 (SH8): EKDAVTSLKNSNSG
(SEQ ID NO: 6)
No. 11 (SH10): EKDAVTSLENSNSG
(SEQ ID NO: 7)
No. 19: NQGTLEFLSNDVSN
(SEQ ID NO: 8)
No. 19 (TKY): NQGTLEFLSNDVST
(SEQ ID NO: 9)
No. 33: LSNKLQGQLKSMGL
(SEQ ID NO: 10)
No. 33 (TKY): LSNKLQDQLKSMGL
(SEQ ID NO: 11)
No. 33 (SH10): FSDKLQNMLKSLNM
(SEQ ID NO: 12)
No. 16: TNGQEVSASIDYNK
(SEQ ID NO: 13)
No. 23: AKLSNFASNDALPD
(SEQ ID NO: 14)
No. 10: PTTSSGASPDSSNP
(SEQ ID NO: 15)
No. 21: GLGRDLFVHSMGDK
(SEQ ID NO: 16)
No. 15: QIGKIKLSDVLSAS
(SEQ ID NO: 17)
No. 34: YGAIDKELHFSGGK
(SEQ ID NO: 18)
No. 20: NVDNILNMPSTTSG
(SEQ ID NO: 19)
No. 22: GNLKGVYYPKSSTT
(SEQ ID NO: 20)
No. 14: ITEKIQSGKLTITI
(SEQ ID NO: 21)
No. 26: FHDFLVSLKGKKFA
(SEQ ID NO: 22)
No. 31: TTGGEVRLFRSFYV
(SEQ ID NO: 23)
No. 35: IGARFGLDYQDINI
(SEQ ID NO: 86)
No. 8: KQLPQPKRSELKPK
(SEQ ID NO: 87)
No. 31N: TNIKQYMQNNHRSQ
(SEQ ID NO: 88)
No. 81: TLTLEGTETFAQNS
(SEQ ID NO: 89)
No. 63: EAYAKNQGDIWSTI
(SEQ ID NO: 90)
No. 73: VIGSKSSITLNSAN
(SEQ ID NO: 91)
No. 61: ADIQSSQTTFANSV

(2) Antibody Preparation

Cysteine (C) was added to the amino terminus of each 14-mer peptide of No. 10, No. 11, No. 16, No. 19, No. 33, or No. 61 among the sequences selected above to synthesize peptides. A rabbit (New Zealand White) was immunized with using keyhole limpet hemocyanin (KLH) as a carrier to obtain an ELISA titer of 1:512,000 or more. The immunized rabbit serum was purified through an affinity column bound with Protein G. A recovered IgG fraction was used as an anti-peptide rabbit antibody (polyclonal antibody) in the following experiment.

(4) Preparation of H. pylori Antigen for ELISA

H. pylori was shake-cultured at a temperature of 37° C. and a humidity of 100% for 48 hours under microaerophilic conditions (5% 02, 10% CO₂, 85% N₂) in brucella broth containing 10% fetal calf serum (FCS). Then, the culture solution was centrifuged to collect bacterial cells. The bacterial cells were washed three times with sterilized distilled water to remove lipopolysaccharide components. A small amount of the bacterial cells was suspended in sterilized distilled water.

(5) Quantification of Protein

Proteins were quantified using Bio-Rad protein assay kit and a 96-well plate. Bovine serum albumin (BSA) was used as a standard protein in calibration curve preparation.

### (Example 3) Measurement of Anti-H. suis Antibody Titer in Infected Subject (Human) Using ELISA (Enzyme-Linked Immunosorbent Assay; Enzyme Immunoassay)

Each peptide dissolved in a 0.2 M carbonate-bicarbonate buffer solution (pH 9.4) or the H. pylori whole cells (4 μg/mL) prepared in Example 2(4) were added dropwise at 100 μL/well to 96-well NUNC-Immuno Plate #439454 and left overnight at 4° C. On the next day, the peptide solution was discarded, and the plate was washed three times with 200 μL/well of PBS-T (phosphate-buffered saline containing 0.05% (V/V) Tween 20, pH 7.4). Nacalai Tesque blocking solution (PBS-based, pH 7.2) diluted 5-fold with sterile distilled water was added dropwise at 200 μL/well and left at 37° C. for 1 hour. The blocking solution was discarded, and the plate was washed three times with 200 μL/well of PBS-T (phosphate-buffered saline containing 0.05% (V/V) Tween 20, pH 7.4).

Serum was collected from each of subjects infected with H. pylori and subjects infected with H. suis. Also, serum was collected as a control from uninfected healthy persons (non-infected). Each human serum diluted using Nacalai Tesque blocking solution (PBS-based, pH 7.2) diluted 10-fold with sterile distilled water was added dropwise at 100 μL/well and left at 37° C. for 1 hour. The serum solution was discarded, and the plate was washed three times with 200 μL/well of PBS-T (phosphate-buffered saline containing 0.05% (V/V) Tween 20, pH 7.4). A horseradish peroxidase-labeled secondary antibody (Goat anti-Human IgG, Jackson ImmunoResearch Inc.) diluted 100,000-fold using Nacalai Tesque blocking solution (PBS-based, pH 7.2) diluted 10-fold with sterile distilled water was added dropwise at 100 μL/well and left at 37° C. for 1 hour. The secondary antibody solution was discarded, and the plate was washed three times with 200 μL/well of PBS-T (phosphate-buffered saline containing 0.05% (V/V) Tween 20, pH 7.4). SuperBlue TMB Microwell Peroxidase Substrate (1-Component) from Kirkegaard & Perry Laboratories, Inc. (KPL) was added dropwise at 100 μL/well for development of blue color, which was then turned into yellow color by the dropwise addition of 1 N hydrochloric acid at 100 μL/well. Absorbance at 450 nm was measured using a plate reader.

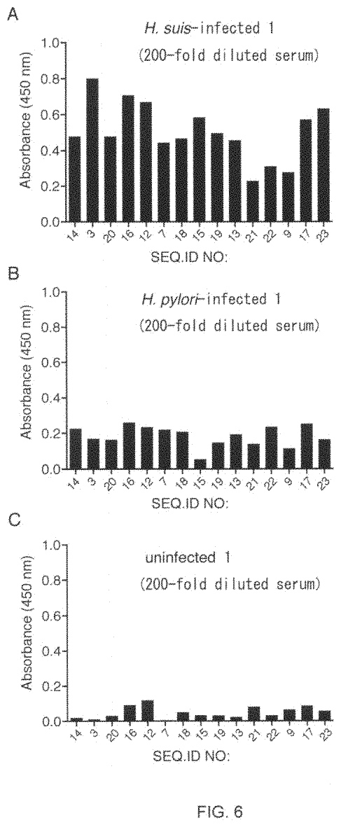

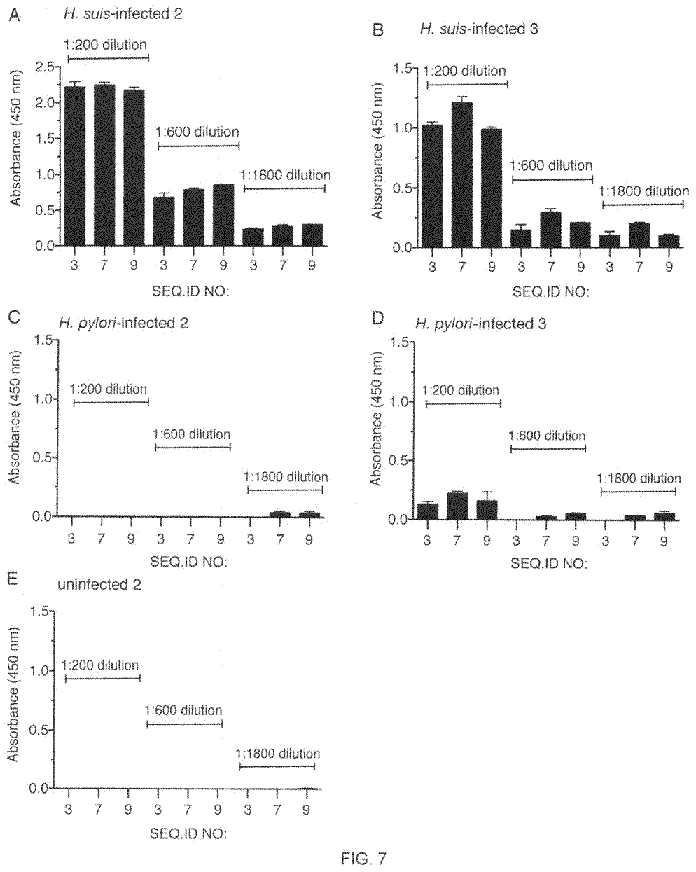

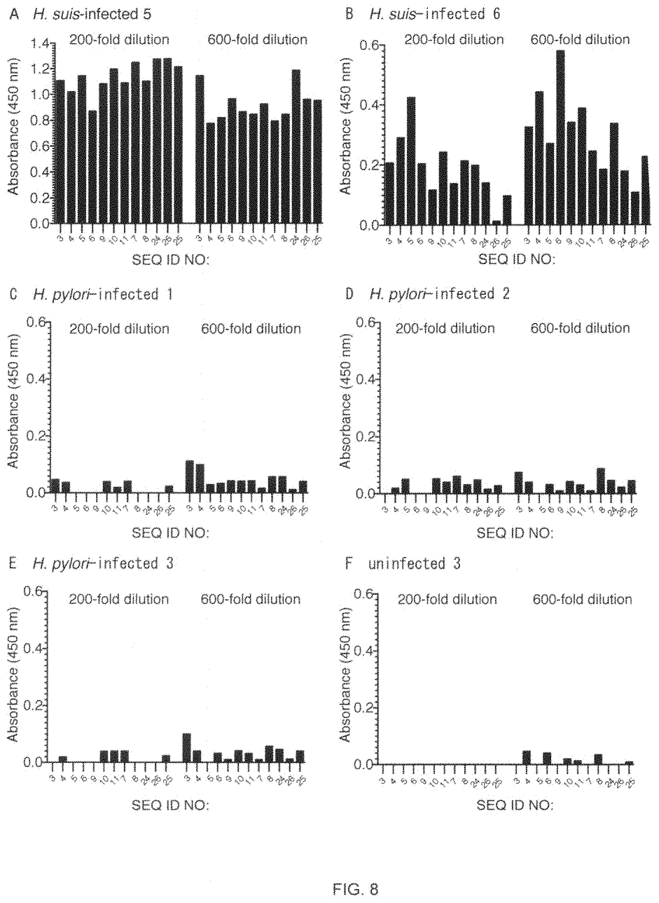

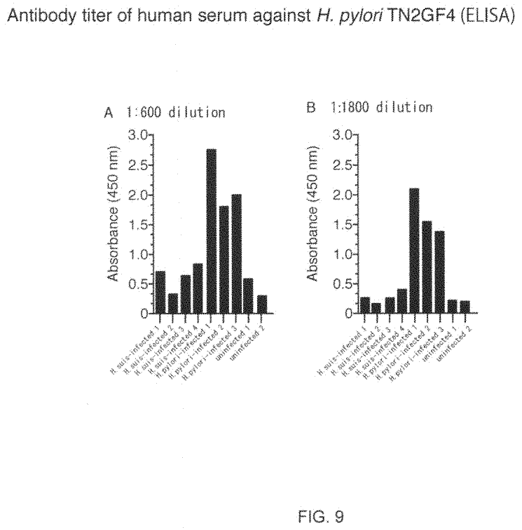

The results of ELISA using the human serum are shown in FIGS. 6 to 9. The H. suis-positive subjects had high antibody titers against the HsvA antigen peptide, whereas the subjects infected with H. pylori and non-infected subjects without H. suis infection had low antibody titers against the HsvA antigen peptide. The antibody titers against the H. pylori whole cells were high in the subjects infected with H. pylori and were low in the subjects infected with H. suis and the non-infected subjects. This showed for the first time that: H. suis expresses the hsvA gene in the human body; and an antibody against the HsvA protein is produced in the blood of a human infected with H. suis. This demonstrated that use of the HsvA antigen peptide enables H. suis infection to be diagnosed by discrimination from H. pylori infection in humans.

### (Example 4) Measurement of Anti-H. suis Antibody Titer in Infected Mouse Using Sandwich ELISA

The antibody against each peptide of Nos. 11, 19, and 33 (1 μg/mL) dissolved in a 0.2 M carbonate-bicarbonate buffer solution (pH 9.4) was added dropwise at 100 μL/well to 96-well NUNC-Immuno Plate #439454 and left overnight at 4° C. On the next day, the antibody solution was discarded, and the plate was washed three times with 200 μL/well of PBS-T (phosphate-buffered saline containing 0.05% (V/V) Tween 20, pH 7.4). Nacalai Tesque blocking solution (phosphate buffer solution-based, pH 7.2) diluted 5-fold with sterile distilled water was added dropwise at 200 μL/well and left at 37° C. for 1 hour. The blocking solution was discarded, and the plate was washed three times with 200 μL/well of PBS-T (phosphate-buffered saline containing 0.05% (V/V) Tween 20, pH 7.4). Each peptide of Nos. 11, 19, and 33 (4 μg/mL) diluted using Nacalai Tesque blocking solution (phosphate buffer solution-based, pH 7.2) diluted 10-fold with sterile distilled water was added dropwise at 100 μL/well and left at 37° C. for 1 hour. The peptide solution was discarded, and the plate was washed three times with 200 μL/well of PBS-T (phosphate-buffered saline containing 0.05% (V/V) Tween 20, pH 7.4). Mouse serum diluted using Nacalai Tesque blocking solution (PBS-based, pH 7.2) diluted 10-fold with exchange water was added dropwise at 100 μL/well and left at 37° C. for 1 hour. The serum solution was discarded, and the plate was washed three times with 200 μL/well of PBS-T (phosphate-buffered saline containing 0.05% (V/V) Tween 20, pH 7.4). A horseradish peroxidase-labeled secondary antibody (Donkey Anti-Mouse IgG, Jackson ImmunoResearch Inc.) diluted 100,000-fold using Nacalai Tesque blocking solution (phosphate buffer solution-based, pH 7.2) diluted 10-fold with sterile distilled water was added dropwise at 100 μL/well and left at 37° C. for 1 hour. The secondary antibody solution was discarded, and the plate was washed three times with 200 μL/well of PBS-T (phosphate-buffered saline containing 0.05% (V/V) Tween 20, pH 7.4). SuperBlue TMB Microwell Peroxidase Substrate (1-Component) from Kirkegaard & Perry Laboratories, Inc. (KPL) was added dropwise at 100 μL/well for development of blue color, which was then turned into yellow color by the dropwise addition of 1 N hydrochloric acid at 100 μL/well. Absorbance at 450 nm was measured using a plate reader.

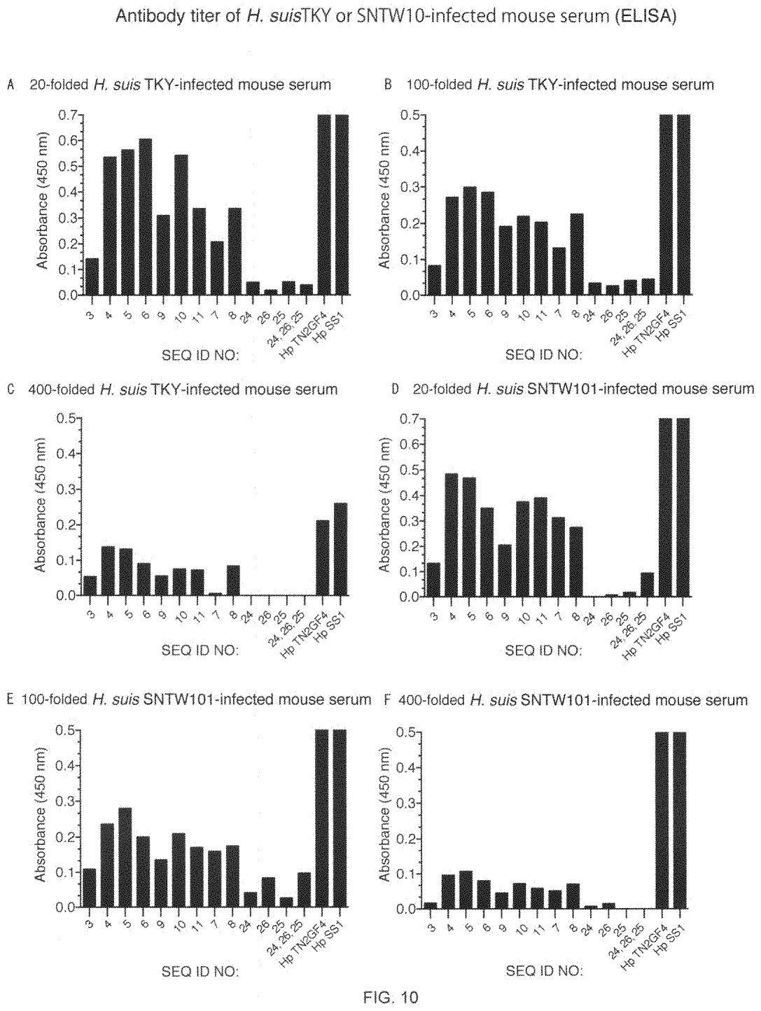

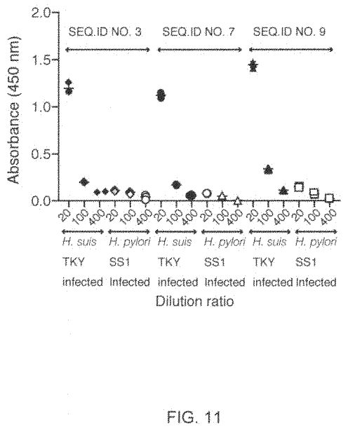

The results of measuring the binding titers of antibodies in the serum of mice infected with an H. suis TKY or H. pylori SS1 strain against the peptides by ELISA and sandwich ELISA are shown in FIGS. 10 and 11, respectively. In both the systems, high antibody titers specific for the HsvA protein-derived peptides were found in the H. suis-infected mouse serum. This showed for the first time that: H. suis expresses the HsvA protein in the mouse body; and an antibody against the HsvA protein is produced in the blood of a mouse infected with H. suis. This demonstrated that use of the HsvA-specific antigen peptide also enables H. suis infection to be diagnosed by discrimination from H. pylori infection in mice.

### (Example 5) Measurement of Specificity of Peptide Antibody Using ELISA (Enzyme-Linked Immunosorbent Assay; Enzyme Immunoassay)

Each peptide of Nos. 11, 19 and 33 (4 μg/mL) dissolved in a 0.2 M carbonate-bicarbonate buffer solution (pH 9.4) or H. pylori whole cells were added dropwise at 100 μL/well to 96-well NUNC-Immuno Plate #439454 and left overnight at 4° C. On the next day, the peptide solution was discarded, and the plate was washed three times with 200 μL/well of PBS-T (phosphate-buffered saline containing 0.05% (V/V) Tween 20, pH 7.4). Nacalai Tesque blocking solution (phosphate buffer solution-based, pH 7.2) diluted 5-fold with sterile distilled water was added dropwise at 200 μL/well and left at 37° C. for 1 hour. The blocking solution was discarded, and the plate was washed three times with 200 μL/well of PBS-T (phosphate-buffered saline containing 0.05% (V/V) Tween 20, pH 7.4). Each anti-peptide antibody (1 μg/mL) obtained in Example 2(2) diluted using Nacalai Tesque blocking solution (phosphate buffer solution-based, pH 7.2) diluted 10-fold with exchange water was added dropwise at 100 μL/well and left at 37° C. for 1 hour. The serum solution was discarded, and the plate was washed three times with 200 μL/well of PBS-T (phosphate-buffered saline containing 0.05% (V/V) Tween 20, pH 7.4). A horseradish peroxidase-labeled secondary antibody (Goat anti-Rabbit IgG, Jackson ImmunoResearch Inc.) diluted 100,000-fold using Nacalai Tesque blocking solution (PBS-based, pH 7.2) diluted 10-fold with exchange water was added dropwise at 100 μL/well and left at 37° C. for 1 hour. The secondary antibody solution was discarded, and the plate was washed three times with 200 μL/well of PBS-T (phosphate-buffered saline containing 0.05% (V/V) Tween 20, pH 7.4). SuperBlue TMB Microwell Peroxidase Substrate (1-Component) from Kirkegaard & Perry Laboratories, Inc. (KPL) was added dropwise at 100 μL/well for development of blue color, which was then turned into yellow color by the dropwise addition of 1 N hydrochloric acid at 100 μL/well. Absorbance at 450 nm was measured using a plate reader.

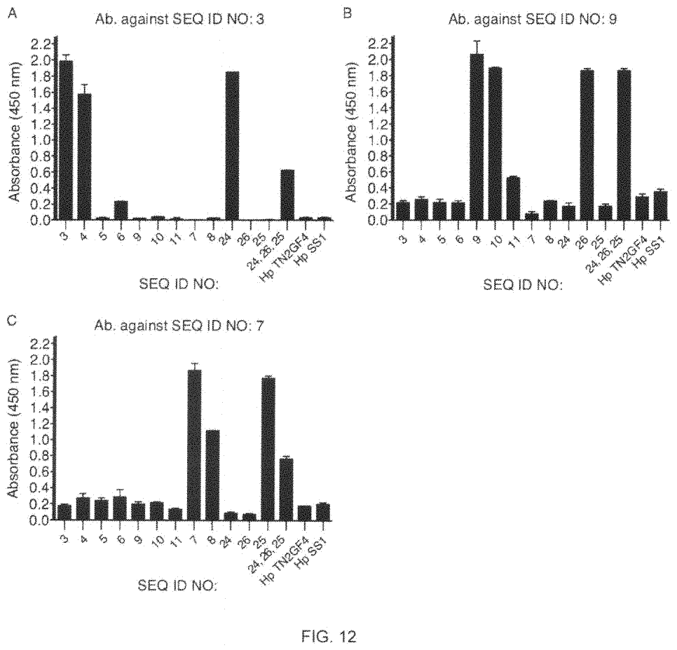

The results of measuring the binding of the anti-peptide rabbit antibodies prepared in Example 2 onto wells bound with each antigen (H. suis HsvA antigen peptide (Nos. 11, 19 and 33) or H. pylori whole cells (TN2GF4 and SS1 strains)) are shown in FIG. 12. The antibody against peptide No. 11 (SEQ ID NO: 3), peptide No. 33 (SEQ ID NO: 9), or peptide No. 19 (SEQ ID NO: 7) specifically exhibited strong binding to the peptide used as antigen in the preparation of this antibody, and on the other hand, exhibited only weak binding to the H. pylori whole cells. Particularly, the antibody against No. 11 peptide exhibited low nonspecific binding strength against the H. pylori whole cells.

### (Example 6) Immunostaining Using Anti-No. 11 Peptide Antibody

A gastric paraffin section of an H. suis-infected human was deparaffinized by dipping in xylene three times, dehydrated ethanol once, 90% ethanol once, 80% ethanol once, and pure water once (5 minutes each) in order. Phosphate-buffered saline containing 1% (W/V) skimmed milk, pH 7.2 was added dropwise to the deparaffinized glass slide and left at room temperature for 30 minutes for blocking treatment. Subsequently, the glass slide was washed three times with phosphate-buffered saline, pH 7.2 at room temperature for 5 minutes. The anti-No. 11 peptide antibody was diluted into 2 μg/mL with phosphate-buffered saline, pH 7.2, added dropwise to the glass slide, and reacted at room temperature for 3 hours. The antibody solution was removed from the glass slide, which was then washed three times with phosphate-buffered saline, pH 7.2 at room temperature for 5 minutes. Alexa-Fluor 488 anti-rabbit IgG (Thermo Fisher Scientific Inc.) (secondary antibody) diluted 400-fold with phosphate-buffered saline, pH 7.2 was added dropwise to the glass slide and reacted at room temperature for 3 hours. The secondary antibody solution was removed from the glass slide, which was then washed three times with phosphate-buffered saline, pH 7.2 at room temperature for 5 minutes. Alexa-Fluor 568 phalloidin (Thermo Fisher Scientific Inc.) diluted 400-fold with phosphate-buffered saline, pH 7.2 was added dropwise to the glass slide and reacted at room temperature for 30 minutes for counterstaining (F actin staining). The secondary antibody solution was removed from the glass slide, which was then washed three times with phosphate-buffered saline, pH 7.2 at room temperature for 5 minutes. The glass slide was mounted to Permaflow (Therma Fisher Scientific Inc.). The glass slide was observed under confocal laser fluorescence microscope Leica TCS-SP5. Photograph A was taken at a 200× magnification. Photograph B was a photograph of the boxed portion of A magnified 760 times. The arrows depict bacterial cells of H. suis.

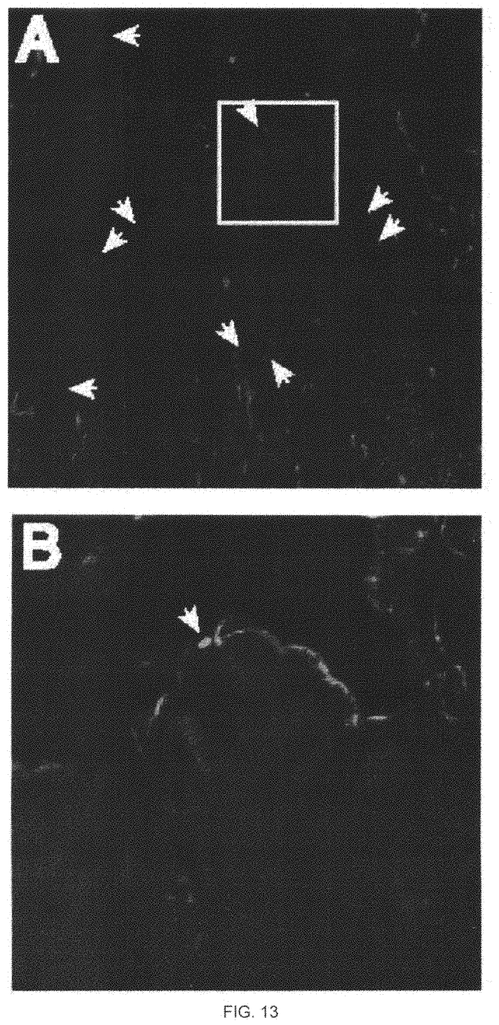

The results are shown in FIG. 13. The bacterial cells in the gastric tissue section of the H. suis-infected patient were stained with the anti-No. 11 peptide antibody. This demonstrated that H. suis infection can be diagnosed by immunohistochemistry using the anti-No. 11 peptide antibody.

### Example 7

Each peptide of peptide Nos. 8, 31N, 81, 63, 73, 61, 11+11 (TKY), 11 (SH8), 11 (SH10), 19+19 (TKY), 33+33 (TKY), 33 (SH10), 16, 23, 10, 21, 20, 22, 31, and 35 prepared in Example 2 or H. pylori TN2GF4 or SS1 whole cells (4 μg/mL) were dissolved in a 0.1 M carbonate-bicarbonate buffer solution (pH 9.4), added dropwise at 100 μL/well to a 96-well plate (NUNC-Immuno plate #439454), and left standing overnight at 4° C. On the next day, the peptide solution was discarded, and the plate was washed three times with 200 μL/well of PBS-T (phosphate-buffered saline containing 0.05% (V/V) Tween 20, pH 7.4). A blocking solution (BSA, 1% (W/V) phosphate buffer solution-based, pH 7.4) was added dropwise at 200 μL/well and left standing at 37° C. for 1 hour. The blocking solution was discarded, and the plate was washed three times with 200 μL/well of PBS-T.

The serum samples used were serum obtained from H. suis-infected patients (n=4), serum obtained from H. pylori-infected patients (n=5), and serum obtained from non-infected subjects (n=3). Each serum was diluted 1,800-fold (diluted 600-fold and 1800-fold only for reaction with H. pylori test specimens) using Nacalai Tesque blocking solution (phosphate buffer solution-based, pH 7.2) diluted 10-fold with distilled water, added dropwise at 50 μL/well to the plate prepared by the method mentioned above, and left standing at 37° C. for 1 hour. The serum solution was discarded, and the plate was washed three times with 200 μL/well of PBS-T. A horseradish peroxidase-labeled secondary antibody (Peroxidase-conjugated AffiniPure Goat Anti-Human IgG (H+L), Jackson ImmunoResearch Inc.) diluted 100,000-fold using Nacalai Tesque blocking solution (phosphate buffer solution-based, pH 7.2) diluted 10-fold with distilled water was added dropwise at 50 μL/well and left standing at 37° C. for 1 hour. The secondary antibody solution was discarded, and the plate was washed three times with PBS-T. SuperBlue TMB Microwell Peroxidase Substrate (1-Component) (Kirkegaard & Perry Laboratories, Inc. (KPL)) was added dropwise at 50 μL/well for development of blue color, which was then turned into yellow color by the dropwise addition of 1 N hydrochloric acid at 50 μL/well. Absorbance at 450 nm (reference: 630 nm) was measured using a plate reader.

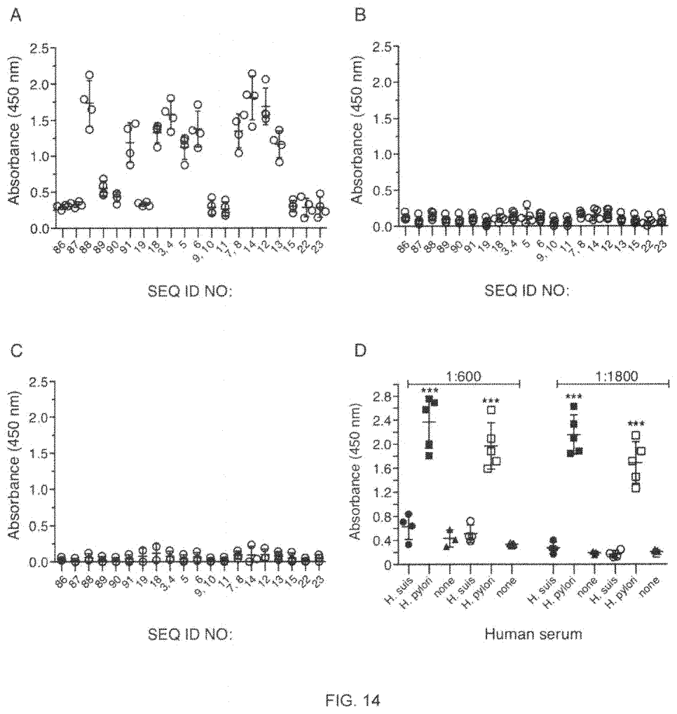

The results are shown in FIG. 14. The present ELISA method was able to discriminate positivity (absorbance: 0.5 or more) from negativity (absorbance: 0.5 or less) as to the human serum (H. suis-positive 4 test specimens (patients 5, 6, 7, and 8); H. pylori-positive 5 test specimens (patients 4, 5, 6, 7, and 8); non-infected 3 test specimens (healthy persons 4, 5, and 6) diluted 1800-fold. Ten peptides of Nos. 81, 61, 20, 11+11 (TKY), 11 (SH8), 11 (SH10), 19+19 (TKY), 10, 16, and 23 were found to have high reactivity with the H. suis-positive patient serum (A). On the other hand, the H. pylori-positive patient serum (B) and the non-infected serum (C) reacted with none of the peptides. The H. pylori-infected serum exhibited strong reactivity with the H. pylori whole cells, whereas the H. suis-infected serum did not exhibit reaction therewith (D). This showed that these peptides are exceedingly specific for antibodies against H. suis without reacting with antibodies against H. pylori. Accordingly, these peptides are useful in specific infection diagnosis using antibodies in serum.

### (Example 8) Specificity of Anti-Peptide Antibody Against Each Peptide of Nos. 61, 11, 33, 19, 10, and 16 for Corresponding Antigen Peptide

Each peptide of Nos. 8, 31N, 81, 63, 73, 61, 11+11 (TKY), 11 (SH8), 11 (SH10), 19+19 (TKY), 33+33 (TKY), 33 (SH10), 16, 23, 10, 21, 20, 22, 31, and 35 prepared in Example 2 was dissolved (4 μg/mL) in a 0.1 M carbonate-bicarbonate buffer solution (pH 9.4), added dropwise at 100 μL/well to a 96-well plate (NUNC-Immuno plate #439454), and left standing overnight at 4° C. On the next day, the peptide solution was discarded, and the plate was washed three times with 200 μL/well of PBS-T (phosphate-buffered saline containing 0.05% (V/V) Tween 20, pH 7.4). A blocking solution (BSA, 1% (W/V) phosphate buffer solution-based, pH 7.4) was added dropwise at 200 μL/well and left standing at 37° C. for 1 hour. The blocking solution was discarded, and the plate was washed three times with 200 μL/well of PBS-T. The anti-peptide rabbit polyclonal antibody (IgG, 0.2 μg/mL) of Example 2 diluted using Nacalai Tesque blocking solution (phosphate buffer solution-based, pH 7.2) diluted 10-fold with distilled water was added dropwise at 50 μL/well and left standing at 37° C. for 1 hour (n=2). The serum solution was discarded, and the plate was washed three times with 200 μL/well of PBS-T. A horseradish peroxidase-labeled secondary antibody (Peroxidase-conjugated AffiniPure Goat Anti-Rabbit IgG (H+L), Jackson ImmunoResearch Inc.) diluted 100,000-fold using Nacalai Tesque blocking solution (phosphate buffer solution-based (pH 7.2) diluted 10-fold with distilled water was added dropwise at 50 μL/well and left standing at 37° C. for 1 hour. The secondary antibody solution was discarded, and the plate was washed three times with PBS-T. SuperBlue TMB Microwell Peroxidase Substrate (1-Component) (Kirkegaard & Perry Laboratories, Inc. (KPL)) was added dropwise at 50 μL/well for development of blue color, which was then turned into yellow color by the dropwise addition of 1 N hydrochloric acid at 50 μL/well. Absorbance at 450 nm (reference: 630 nm) was measured using a plate reader.

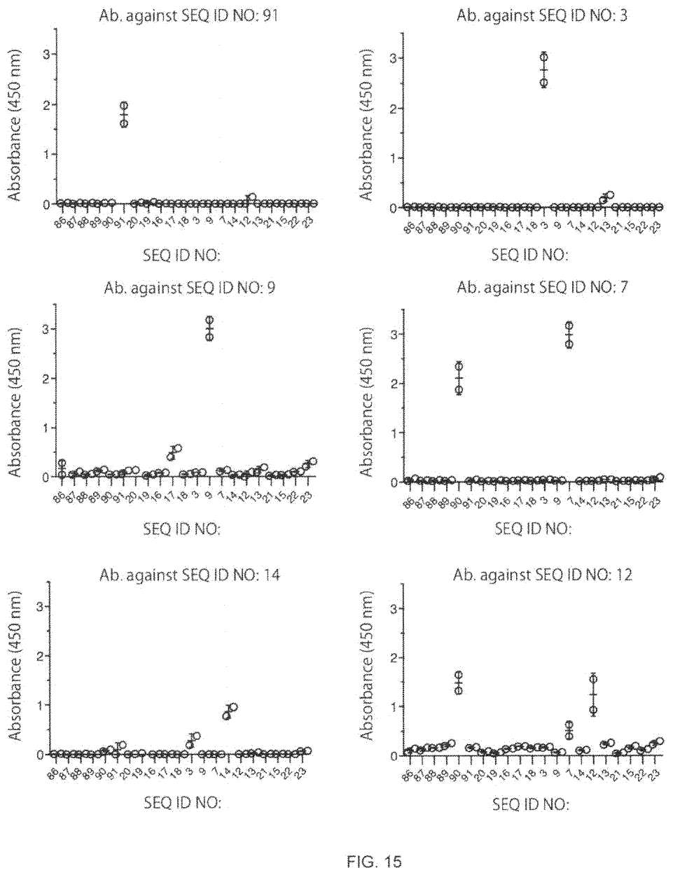

The results are shown in FIG. 15. The antibody against each peptide was shown to specifically react with only the corresponding peptide used as an antigen.

### (Example 9) Specificity of Anti-Peptide Antibody Against Each Peptide of Nos. 61, 11, 33, 19, 10, and 16 for Bacterial Cells

H. pylori SS1, H. pylori TN2GF4, H. pylori NCTC11637, H. pylori TY1289, H. pylori RC-1, H. pylori TK1029, H. pylori TY281, H. pylori ATCC43579, H. pylori TK1081, or H. suis SNTW101 (4 μg/mL) dissolved in a 0.1 M carbonate-bicarbonate buffer solution (pH 9.4) was added dropwise at 100 μL/well to a 96-well plate (NUNC-Immuno plate #439454), and left standing overnight at 4° C. On the next day, the peptide solution was discarded, and the plate was washed three times with 200 μL/well of PBS-T (phosphate-buffered saline containing 0.05% (V/V) Tween 20, pH 7.4). A blocking solution (BSA, 1% (W/V) phosphate buffer solution-based, pH 7.4) was added dropwise at 200 μL/well and left standing at 37° C. for 1 hour. The blocking solution was discarded, and the plate was washed three times with 200 μL/well of PBS-T. The anti-peptide rabbit polyclonal antibody (IgG, 0.2 μg/mL) prepared in Example 2 or a polyclonal antibody against H. pylori SS1 (rabbit IgG) (described in Non Patent Literature 4) diluted using Nacalai Tesque blocking solution (phosphate buffer solution-based, pH 7.2) diluted 10-fold with distilled water was added dropwise at 50 μL/well and left standing at 37° C. for 1 hour (n=2 to 4). The serum solution was discarded, and the plate was washed three times with 200 μL/well of PBS-T. A horseradish peroxidase-labeled secondary antibody (Peroxidase-conjugated AffiniPure Goat Anti-Rabbit IgG (H+L), Jackson ImmunoResearch Inc.) diluted 100,000-fold using Nacalai Tesque blocking solution (phosphate buffer solution-based, pH 7.2) diluted 10-fold with distilled water was added dropwise at 50 μL/well and left standing at 37° C. for 1 hour. The secondary antibody solution was discarded, and the plate was washed three times with PBS-T. SuperBlue TMB Microwell Peroxidase Substrate (1-Component) (Kirkegaard & Perry Laboratories, Inc. (KPL)) was added dropwise at 50 μL/well for development of blue color, which was then turned into yellow color by the dropwise addition of 1 N hydrochloric acid at 50 μL/well. Absorbance at 450 nm (reference: 630 nm) was measured using a plate reader.

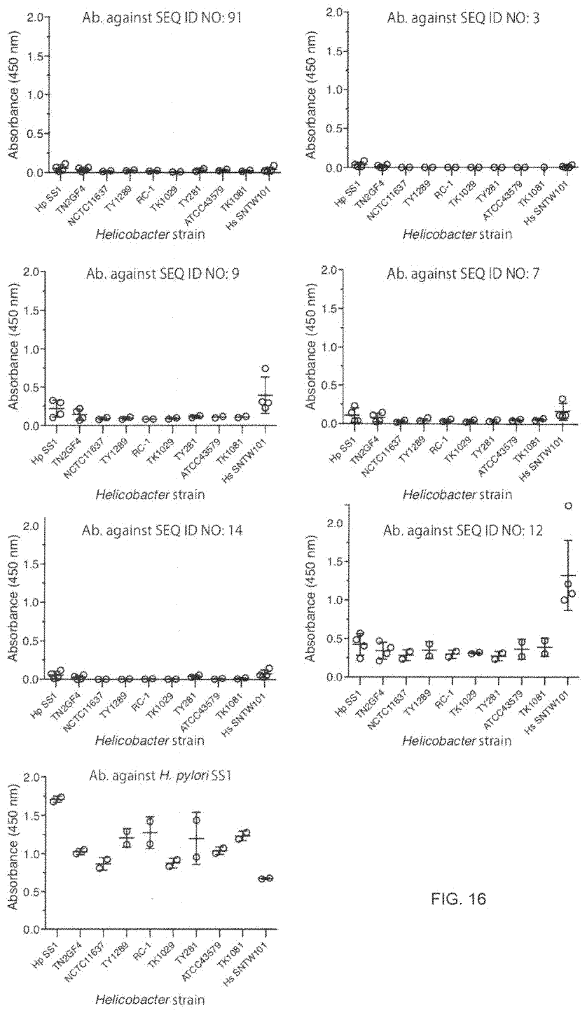

The results are shown in FIG. 16. The anti-peptide antibody did not react with the bacterial cells of H. pylori. On the other hand, the anti-H. pylori polyclonal antibody reacted with the bacterial cells of H. pylori and H. suis.

### (Example 10) Method for Culturing H. suis SNTW101

(1) Separation of H. suis SNTW101 from Gastric Mucosa of Infected Mouse and Culture

Culture was performed using a modified medium based on the method of Smet A., et al., International Journal of Systematic and Evolutionary Microbiology (2012), 62, 299-306. 0.05% (V/V) hydrochloric acid and 10 mg/L linezolid were added to Brucella agar plate (2 vials/L Skirrow, 10 mg/L vancomycin, 5 mg/L trimethoprim, 2500 IU/L polymyxin B, 2 vials/L vitox, 5 mg/L amphotericin B, 20% (V/V) fetal calf serum). Shake culture was performed for 2 weeks under conditions of 37° C., a humidity of 100%, 12% CO₂, 5% 02, and 83% N₂. 1504 of Brucella broth (2 vials/L Skirrow, 2 vials/L vitox, 5 mg/L linezolid, 20% (V/V) fetal calf serum, pH 5) was added thereto every other day. The culture solution was centrifuged, and the precipitates were washed three times with water and used as bacterial cells of H. suis SNTW101. Proteins were quantified using Bio-Rad protein assay kit. A calibration curve was prepared using bovine serum albumin (BSA) as a standard protein.

### (Example 11) Immunohistochemistry Using Anti-No. 16 Peptide (SEQ ID NO: 12) Antibody

The stomach of an H. suis SNTW101-infected C57BL/6 mouse was cut open along the greater curvature. The contents were washed with PBS, and the stomach was excised in the perpendicular direction and fixed in 10% neutral buffered formalin. A gastric tissue was embedded in paraffin, and 4 μm sections were prepared. The sections were deparaffinized, then washed with water, and heat-treated at 95° C. for 20 minutes using a citrate buffer solution (pH 6.0) for antigen retrieval. The sections were washed with water and then reacted with 0.3% hydrogen peroxide water at room temperature for 10 minutes. The sections were washed twice with PBS and then reacted with the anti-No. 16 peptide antibody (2 μg/mL PBS) at room temperature for 1 hour. The sections were washed twice with PBS and then reacted with Histofine Simple Stain Mouse MAX-PO® (Nichirei Corp., Code. 414341) as a secondary antibody at room temperature for 30 minutes. The sections were washed twice with PBS, followed by color development at room temperature for 5 minutes using a DAB (3,3′-diaminobenzidine tetrahydrochloride) solution. The sections were washed twice with PBS, then poststained with hematoxylin, washed with water, dehydrated, immersed, and mounted for microscopic observation.

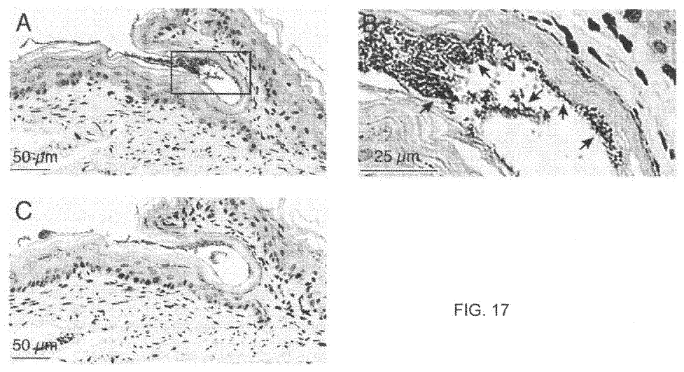

The results are shown in FIG. 17. The antibody against No. 16 peptide (SEQ ID NO: 12) was shown to be able to immunostain bacterial cells of H. suis.

1.-24. (canceled)

25. A protein consisting of the amino acid sequences set forth in SEQ ID NO: 2, or a protein consisted of the amino acids of positions 28-2992 in the amino acid sequences set forth in SEQ ID NO: 2.

26. A method for determining infection of a subject with H. suis, comprising:
detecting the HsvA protein or the fragment thereof in a sample derived from the subject,
determining that H. suis is present in the sample in which the HsvA protein or the fragment thereof have been detected, and
determining the subject is infected with H. suis, when the sample derived from whom is determined that H. suis is present by the method; or
detecting an antibody that binds to an HsvA antigen peptide in a blood sample derived from the subject; and
determining the subject is infected with H. suis when the antibody that binds to the peptide has been detected.

27. The method for determining infection of claim 26, comprising:
contacting the sample derived from the subject with an antibody or an immunoreactive fragment thereof, that specifically binds to HsvA antigen peptide derived from H. suis;
detecting the HsvA protein in the sample bound with the antibody or the immunoreactive fragment thereof; and
determining that H. suis is present in the sample when the HsvA protein bound with the antibody or the immunoreactive fragment thereof has been detected.

28. The method for determining infection of claim 26, wherein the HsvA protein consists of an amino acid sequence that is specific to H. suis, and that does not exist in H. pylori.

29. The method for determining infection with H. suis of claim 26, comprising:
contacting the blood sample derived from the subject with the peptide selected from a group consisting of:
the HsvA antigen peptide having a sequence comprising 5 to 50 amino acids contained in any one of the amino acid sequences set forth in SEQ ID NO: 2, SEQ ID NO: 28, SEQ ID NO: 30, or SEQ ID NOs: 78 to 83 or any one of the sequences:
(peptide No. 11: SEQ ID NO: 24)
EKX1AVX2X3X4X5NSNX6X7;
(peptide No. 11: SEQ ID NO: 3)
EKKAVQQMENSNPD,
(peptide No. 11 (TKY): SEQ ID NO: 4)
EKKAVEQMENSNPD,
(peptide No. 11 (SH8): SEQ ID NO: 5)
EKDAVTSLKNSNSG,
(peptide No. 11 (SH10): SEQ ID NO: 6)
EKDAVTSLENSNSG,
(peptide No. 19: SEQ ID NO: 25)
NQGTLEFLSNDVSX8;
(peptide No. 19: SEQ ID NO: 7)
NQGTLEFLSNDVSN,
(peptide No. 19 (TKY): SEQ ID NO:8)
NQGTLEFLSNDVST,
(peptide No. 33: SEQ ID NO: 26)
X9SX10KLQX11X12LKSX13X14X15;
(peptide No. 33: SEQ ID NO: 9)
LSNKLQGQLKSMGL,
(peptide No. 33 (TKY): SEQ ID NO: 10)
LSNKLQDQLKSMGL,
(peptide No. 33 (SH10): SEQ ID NO: 11)
FSDKLQNMLKSLNM,
(peptide No. 16: SEQ ID NO: 12)
TNGQEVSASIDYNK;
(peptide No. 23: SEQ ID NO: 13)
AKLSNFASNDALPD;
(peptide No. 10: SEQ ID NO: 14)
PTTSSGASPDSSNP;
(peptide No. 21: SEQ ID NO: 15)
GLGRDLFVHSMGDK;
(peptide No. 15: SEQ ID NO: 16)
QIGKIKLSDVLSAS;
(peptide No. 34: SEQ ID NO: 17)
YGAIDKFLHFSGGK;
(peptide No. 20: SEQ ID NO: 18)
NVDNILNMPSTTSG;
(peptide No. 22: SEQ ID NO: 19)
GNLKGVYYPKSSTT;
(peptide No. 14: SEQ ID NO: 20)
ITEKIQSGKLTITI;
(peptide No. 26: SEQ ID NO: 21)
FHDFLVSLKGKKFA;
(peptide No. 31: SEQ ID NO: 22)
TTGGEVRLFRSFYV;
(peptide No. 35: SEQ ID NO: 23)
IGARFGLDYQDINI;
(peptide No. 8: SEQ ID NO: 86)
KQLPQPKRSELKPK;
(peptide No. 31N: SEQ ID NO: 87)
TNIKQYMQNNHRSQ;
(peptide No. 81: SEQ ID NO: 88)
TLTLEGTETFAQNS;
(peptide No. 63: SEQ ID NO: 89)
EAYAKNQGDIWSTI;
(peptide No. 73: SEQ ID NO: 90)
VIGSKSSITLNSAN;
and
(peptide No. 61: SEQ ID NO: 91)
ADIQSSQTTFANSV,
wherein X1 is K or D; X2 is Q, E or T; X3 is Q or S; X4 is M or L; X5 is E or K; X6 is P or S; X7 is D or G; X8 is N or T; X9 is L or F; X10 is N or D; X11 is G, D or N; X12 is Q or M; X13 is M or L; X14 is G or N; and X15 is L or M;
detecting an antibody that binds to said peptide in the blood sample; and
determining the subject is infected with H. suis when the antibody that binds to the peptide has been detected.

30. A method for treatment of H. suis infection comprising: administering any one selected from a group consisting of:
the HsvA antigen peptide derived from H. suis having a sequence consisting of 5 to 50 amino acids contained in any one of the amino acid sequences set forth in SEQ ID NO: 2, SEQ ID NO: 28, SEQ ID NO: 30, or SEQ ID NOs: 78 to 83;
a nucleic acid molecule consisting of 5 to 40 nucleotides, wherein the nucleic acid molecule is capable of binding to an hsvA gene under stringent conditions; and
an antibody or an immunoreactive fragment thereof, that specifically binds to the HsvA antigen peptide derived from H. suis having a sequence consisting of 5 to 50 amino acids contained in any one of the amino acid sequences set forth in SEQ ID NO: 2, SEQ ID NO: 28, SEQ ID NO: 30, or SEQ ID NOs: 78 to 83.

31. The method of claim 30 for treatment or prevention of gastritis, gastric ulcer, duodenal ulcer, stomach cancer, chronic gastritis, gastric MALT lymphoma, nodular gastritis, idiopathic thrombocytopenic purpura, functional dyspepsia, or diffuse large B-cell lymphoma.

32. The method of claim 26, wherein the antibody is an antibody that specifically binds to the HsvA antigen peptide derived from H. suis having a sequence consisting of 5 to 50 amino acids contained in any one of the amino acid sequences set forth in SEQ ID NO: 2, SEQ ID NO: 28, SEQ ID NO: 30, or SEQ ID NOs: 78 to 83.

33. The method for determining infection of claim 26, wherein H. pylori infection is not detected by the method, and/or, wherein the HsvA antigen peptide is the peptide having a sequence consisting of 5 to 50 amino acids contained in any one of the amino acid sequences set forth in SEQ ID NO: 2, SEQ ID NO: 28, SEQ ID NO: 30, or SEQ ID NOs: 78 to 83.

34. The method for determining infection of claim 33, wherein the HsvA antigen peptide has any one of amino acid sequence selected from a group consisting of:
(peptide No. 11: SEQ ID NO: 24)
EKX1AVX2X3X4X5NSNX6X7;
(peptide No. 19: SEQ ID NO: 25)
NQGTLEFLSNDVSX8;
(peptide No. 33: SEQ ID NO: 26)
X9SX10KLQX11X12LKSX13X14X15;
(peptide No. 16: SEQ ID NO: 12)
TNGQEVSASIDYNK;
(peptide No. 23: SEQ ID NO: 13)
AKLSNFASNDALPD;
(peptide No. 10: SEQ ID NO: 14)
PTTSSGASPDSSNP;
(peptide No. 21: SEQ ID NO: 15)
GLGRDLFVHSMGDK;
(peptide No. 15: SEQ ID NO: 16)
QIGKIKLSDVLSAS;
(peptide No. 34: SEQ ID NO: 17)
YGAIDKFLHFSGGK;
(peptide No. 20: SEQ ID NO: 18)
NVDNILNMPSTTSG;
(peptide No. 22: SEQ ID NO: 19)
GNLKGVYYPKSSTT;
(peptide No. 14: SEQ ID NO: 20)
ITEKIQSGKILTITI;
(peptide No. 26: SEQ ID NO: 21)
FHDFLVSLKGKKFA;
(peptide No. 31: SEQ ID NO: 22)
TTGGEVRLFRSFYV;
(peptide No. 35: SEQ ID NO: 23)
IGARFGLDYQDINI;
(peptide No. 8: SEQ ID NO: 86)
KQLPQPKRSELKPK;
(peptide No. 31N: SEQ ID NO: 87)
TNIKQYMQNNHRSQ;
(peptide No. 81: SEQ ID NO: 88)
TLTLEGTETFAQNS;
(peptide No. 63: SEQ ID NO: 89)
EAYAKNQGDIWSTI;
(peptide No. 73: SEQ ID NO: 90)
VIGSKSSITLNSAN;
and
(peptide No. 61: SEQ ID NO: 91)
ADIQSSQTTFANSV;
wherein X1 is K or D; X2 is Q, E or T; X3 is Q or S; X4 is M or L; X5 is E or K; X6 is P or S; X7 is D or G; X8 is N or T; X9 is L or F; X10 is N or D; X11 is G, D or N; X12 is Q or M; X13 is M or L; X14 is G or N; and X15 is L or M.

35. The method for determining infection of claim 33, wherein the HsvA antigen peptide has any one of amino acid sequence selected from a group consisting of:
(peptide No. 11: SEQ ID NO: 3)
EKKAVQQMENSNPD,
(peptide No. 11 (TKY): SEQ ID NO: 4)
EKKAVEQMENSNPD,
(peptide No. 11 (SH8): SEQ ID NO: 5)
EKDAVTSLKNSNSG,
(peptide No. 11 (SH10): SEQ ID NO: 6)
EKDAVTSLENSNSG,
(peptide No. 19: SEQ ID NO: 25)
NQGTLEFLSNDVSX8;
(peptide No. 19: SEQ ID NO: 7)
NQGTLEFLSNDVSN,
(peptide No. 19 (TKY): SEQ ID NO:8)
NQGTLEFLSNDVST,
(peptide No. 33: SEQ ID NO: 9)
LSNKLQGQLKSMGL,
(peptide No. 33 (TKY): SEQ ID NO: 10)
LSNKLQDQLKSMGL,
(peptide No. 33 (SH10): SEQ ID NO: 11)
FSDKLQNMLKSLNM,
(peptide No. 16: SEQ ID NO: 12)
TNGQEVSASIDYNK;
(peptide No. 23: SEQ ID NO: 13)
AKLSNFASNDALPD;
(peptide No. 10: SEQ ID NO: 14)
PTTSSGASPDSSNP;
(peptide No. 21: SEQ ID NO: 15)
GLGRDLFVHSMGDK;
(peptide No. 15: SEQ ID NO: 16)
QIGKIKLSDVLSAS;
(peptide No. 34: SEQ ID NO: 17)
YGAIDKFLHFSGGK;
(peptide No. 20: SEQ ID NO: 18)
NVDNILNMPSTTSG;
(peptide No. 22: SEQ ID NO: 19)
GNLKGVYYPKSSTT;
(peptide No. 14: SEQ ID NO: 20)
ITEKIQSGKLTITI;
(peptide No. 26: SEQ ID NO: 21)
FHDFLVSLKGKKFA;
(peptide No. 31: SEQ ID NO: 22)
TTGGEVRLFRSFYV;
(peptide No. 35: SEQ ID NO: 23)
IGARFGLDYQDINI;
(peptide No. 8: SEQ ID NO: 86)
KQLPQPKRSELKPK;
(peptide No. 31N: SEQ ID NO: 87)
TNIKQYMQNNHRSQ;
(peptide No. 81: SEQ ID NO: 88)
TLTLEGTETFAQNS;
(peptide No. 63: SEQ ID NO: 89)
EAYAKNQGDIWSTI;
(peptide No. 73: SEQ ID NO: 90)
VIGSKSSITLNSAN;
and
(peptide No. 61: SEQ ID NO: 91)
ADIQSSQTTFANSV.

36. The method for determining infection of claim 33, wherein the HsvA antigen peptide has any one of amino acid sequence selected from a group consisting of:
(peptide No. 11: SEQ ID NO: 3)
EKKAVQQMENSNPD,
(peptide No. 11 (TKY): SEQ ID NO: 4)
EKKAVEQMENSNPD,
(peptide No. 11 (SH8): SEQ ID NO: 5)
EKDAVTSLKNSNSG,
(peptide No. 11 (SH10): SEQ ID NO: 6)
EKDAVTSLENSNSG,
and
(peptide No. 16: SEQ ID NO: 12)
TNGQEVSASIDYNK.

37. The method for determining infection of claim 33, wherein the HsvA antigen peptide has an amino acid sequence of EKKAVQQMENSNPD (peptide No. 11: SEQ ID NO: 3) or TNGQEVSASIDYNK (peptide No. 16: SEQ ID NO: 12).
